(19) United States
(12) Patent Application Publication
(10) Pub. No.: US 2023/0159979 A1
(43) Pub. Date: May 25, 2023

(54) TRANSDUCERS, NANOPARTICLE TRANSDUCER DEVICES AND SYSTEMS, AND RELATED METHODS OF USE

(71) Applicants: UNIVERSITY OF WASHINGTON, Seattle, WA (US); LAMPROGEN, INC., Seattle, WA (US)

(72) Inventors: Daniel T. Chiu, Seattle, WA (US); Haobin Chen, Seattle, WA (US); Jiangbo Yu, Seattle, WA (US)

(73) Assignees: UNIVERSITY OF WASHINGTON, Seattle, WA (US); LAMPROGEN, INC., Seattle, WA (US)

(21) Appl. No.: 17/904,916

(22) PCT Filed: Apr. 15, 2021
(86) PCT No.: PCT/US2021/027454

§ 371 Date: Aug. 24, 2022

Related U.S. Application Data
(60) Provisional application No. 63/012,002, filed on Apr. 17, 2020.

(51) Int. Cl. C12Q 1/32; C12Q 1/00; C09K 11/06; C09K 11/02

(52) U.S. Cl. CPC C12Q 1/32; C12Q 1/008; C09K 11/06; C09K 11/025; B82Y 20/00

(57) ABSTRACT

Transducers, kits, systems, and methods for determining a concentration of an analyte are described. In an embodiment, the transducers include a chromophore; and an enzyme physically associated with the chromophore. In an embodiment, the transducer is configured to catalyze a reaction comprising a plurality of reaction elements. In an embodiment, the plurality of reaction elements comprises one or more reactants including the analyte and one or more products. In an embodiment, an amount of fluorescence emitted from the chromophore is determined by a concentration of a reaction element of the plurality of reaction elements.

### CROSS-REFERENCES TO RELATED APPLICATIONS

This application is a National Stage of International Patent Application No. PCT/US2021/027454, filed on Apr. 15, 2021, which claims the benefit of U.S. Provisional Patent Application No. 63/012,002, filed Apr. 17, 2020, the contents of which is are hereby incorporated by reference in its entirety.

### BACKGROUND

Monitoring bodily fluid metabolite levels is an important part of managing diseases and injuries. Examples include phenylalanine for assessing phenylketonuria (PKU), glucose for managing diabetes, leucine for monitoring maple syrup urine disease, lactic acid for assessing tissue oxygenation, tyrosine for detecting tyrosinemia, glutamate for assessing ischemic strokes, and α-ketoglutarate for monitoring nonalcoholic fatty liver disease (NAFLD). Biosensors for the measurement of metabolites of interest have significantly improved the quality of life of both the patient and caregivers. In recent years, the demand has grown in the field of medical diagnostics and healthcare management for reliable, easy-to-use, and cost-efficient metabolite monitors suitable for use at the point of care (POC). However, technical hurdles and challenges still impede the commercialization of some disease-related metabolite biosensors. Such challenges can include including multiplexed monitoring, specificity, portability, operability, and long-term stability.

Recently, semiconducting polymer dots (Pdots) have attracted considerable attention in the interdisciplinary studies of materials science, biology, and medicine. Compared with small fluorescent dyes and inorganic semiconducting quantum dots (Qdots), Pdots demonstrate highly desirable characteristics, including high brightness, fast emission rate, large absorption cross section, excellent photostability, nontoxicity, and versatile surface modification features. Such outstanding properties make them widely used in cellular labeling, in vivo imaging, single-particle tracking, drug/gene delivery, and tumor therapy. In addition, small Pdot-based biosensors have been developed, including ones for pH, temperature, metal ions, oxygen, and glucose.

However, to the best of our knowledge, conventional monitoring of reactions using Pdots has required covalent bonding between an enzyme and a Pdot. Such covalent bonding limits how Pdots are made and deployed in determining analyte concentrations.

### SUMMARY

To address these and related challenges, the present disclosure provides transducers, such as nanoparticle transducers, nanoparticle transducer devices and systems, and related methods of use. In certain embodiments and as discussed in further detail herein, enzymes are physically associated with Pdots and/or chromophoric polymers, such as when the enzymes and Pdots and/or enzymes and chromophoric polymers are dispersed in a common solvent, coupled to a common substrate, coupled together, encapsulated together in a hydrogel bead, and the like.

Accordingly, in an aspect, the present disclosure provides a nanoparticle transducer for analyte concentration measurements, the nanoparticle transducer comprising: a nanoparticle comprising a chromophore; and an NADH-dependent or NADPH-dependent enzyme coupled to the nanoparticle and configured to catalyze a reaction comprising a plurality of reaction elements; wherein the plurality of reaction elements comprises one or more reactants including the analyte and one or more products, and wherein an amount of fluorescence emitted from the chromophore is determined by a concentration of a reaction element of the plurality of reaction elements.

In another aspect, the present disclosure provides a transducer substrate for analyte concentration measurements, the transducer substrate comprising: a nanoparticle comprising a chromophore coupled to a substrate; and an enzyme coupled to the substrate and configured to catalyze a reaction comprising a plurality of reaction elements; wherein the plurality of reaction elements comprises one or more reactants including the analyte and one or more products, and wherein an amount of fluorescence emitted from the chromophore is determined by a concentration of a reaction element of the plurality of reaction elements.

In yet another aspect, the present disclosure provides a kit for analyte concentration measurements, the kit comprising: a nanoparticle comprising a chromophore; and an enzyme physically associated with the nanoparticle and configured to catalyze a reaction comprising a plurality of reaction elements; wherein the plurality of reaction elements comprises one or more reactants including the analyte and one or more products, and wherein an amount of fluorescence emitted from the chromophore is determined by a concentration of a reaction element of the plurality of reaction elements.

In another aspect, the present disclosure provides a transducer for analyte concentration measurements, the transducer comprising: a chromophore including a semiconducting chromophoric polymer; and an enzyme physically associated with the semiconducting chromophoric polymer and configured to catalyze a reaction comprising a plurality of reaction elements; wherein the plurality of reaction elements comprises one or more reactants including the analyte and one or more products, and wherein an amount of fluorescence emitted from the chromophore is determined by a concentration of a reaction element of the plurality of reaction elements.

In an aspect, the present disclosure provides a system for analyte concentration measurements, the system comprising: a nanoparticle transducer according to any embodiment described herein, a transducer substrate according to any embodiment described herein, a kit according to any embodiment described herein, or a transducer according to any embodiment described herein; an illumination source configured to illuminate the chromophore of the nanoparticle transducer, the transducer substrate, the kit, or the transducer to induce fluorescence therefrom; a photodetector configured to generate a signal based on the fluorescence from the chromophore; and a controller operatively coupled to the illumination source and the photodetector and including logic, that when executed by the controller, causes the system to perform operations including: illuminating the chromophore with the illumination source; and determining a concentration of the analyte based upon the signal from the photodetector.

In an aspect, the present disclosure provides a method of measuring a concentration of an analyte in a fluid, the method comprising: contacting the fluid with a Pdot including a chromophore and an NADH-dependent or NADPH-dependent enzyme coupled to the Pdot configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements; illuminating the Pdot to induce fluorescence therefrom; measuring the fluorescence from the Pdot; and determining the concentration of the analyte based on the measured fluorescence.

In an aspect, the present disclosure provides a method of measuring a concentration of an analyte in a fluid, the method comprising: contacting the fluid with a Pdot including a chromophore and an enzyme physically associated with the Pdot configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements; illuminating the Pdot to induce fluorescence therefrom; measuring the fluorescence from the Pdot; and determining the concentration of the analyte based on the measured fluorescence. In an embodiment, the fluorescence emitted from the chromophore defines a fluorescence ratio equal to a ratio of an amount of fluorescence emitted at a signal fluorescence wavelength to an amount of fluorescence emitted at a control fluorescence wavelength. In an embodiment, the fluorescence emitted from the one or more chromophores defines a fluorescence ratio equal to a ratio of an amount of fluorescence emitted at a signal fluorescence wavelength to an amount of fluorescence emitted at a control fluorescence wavelength. In an embodiment, the fluorescence ratio is determined by the concentration of the fluid component or fluid constituent.

In an aspect, the present disclosure provides a method of measuring a concentration of an analyte in a fluid, the method comprising: contacting the fluid with chromophore including a semiconducting chromophoric polymer and an enzyme physically associated with the chromophore configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements; illuminating the chromophore to induce fluorescence therefrom; measuring the fluorescence from the chromophore; and determining the concentration of the analyte based on the measured fluorescence.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a transmission electron microscopy (TEM) image of PFBT Pdots, in accordance with an embodiment of the disclosure;

FIG. 1B graphically illustrates a size distribution of the Pdots of FIG. 1A measured by dynamic light scattering (DLS), in accordance with an embodiment of the disclosure;

FIG. 1C graphically illustrates the Zeta-potential of six different Pdots, from left to right: PFO, PDHF, PFBT, PFBTTBT, PFTBT, and DPA-CNPPV Pdots, in accordance with an embodiment of the disclosure;

FIG. 1D includes photographs of Pdot solutions, in accordance with embodiments of the disclosure, under white light (upper) and 365 nm ultraviolet light illumination (lower);

FIGS. 1E and 1F graphically illustrate absorption spectra (1E) and emission (1F) spectra of PFO, PDHF, PFBT, PFBTTBT, PFTBT, and DPA-CNPPV Pdots, in accordance with an embodiment of the disclosure;

FIG. 2A graphically illustrates fluorescence of PFO Pdots with increasing NADH concentration obtained by exciting at λₑₓ=380 nm, in accordance with an embodiment of the disclosure;

FIG. 2B graphically illustrates fluorescence of PDHF Pdots with increasing NADH concentration obtained by exciting at λₑₓ=380 nm, in accordance with an embodiment of the disclosure;

FIG. 2C graphically illustrates fluorescence of PFBT Pdots with increasing NADH concentration obtained by exciting at λₑₓ=330 nm, in accordance with an embodiment of the disclosure;

FIG. 2D graphically illustrates fluorescence of PFBTTBT Pdots with increasing NADH concentration obtained by exciting at λₑₓ=380 nm, in accordance with an embodiment of the disclosure;

FIG. 2E graphically illustrates fluorescence of PFTBT Pdots with increasing NADH concentration obtained by exciting at λₑₓ=380 nm, in accordance with an embodiment of the disclosure;

FIG. 3A is emission spectra of DPA-CNPPV Pdots (λₑₓ=385 nm), in accordance with an embodiment of the disclosure, in the presence of physiologically relevant NADH range (0-2 mM);

FIG. 3B graphically illustrates a ratiometric calibration plot (R/R₀; R=1458 nm/1627 nm; R₀ indicates the fluorescence intensity ratio of Pdots of FIG. 3A, in accordance with an embodiment of the disclosure;

FIG. 3C graphically illustrates emission spectra of DPA-CNPPV Pdots (λₑₓ=385 nm) of FIG. 3A in the presence of higher NADH concentrations (2-10 mM), in accordance with an embodiment of the disclosure;

FIG. 3D graphically illustrates a ratiometric calibration plot (R/R₀; R=1458 nm/1627 nm; R₀ indicates the fluorescence intensity ratio of Pdots of FIG. 3C, in accordance with an embodiment of the disclosure;

FIG. 3E graphically illustrates photostability of DPA-CNPPV Pdots of FIG. 3A under the excitation of 385 nm light for 30 min, in accordance with an embodiment of the disclosure;

FIG. 3F graphically illustrates a response curve of the DPA-CNPPV Pdots of FIG. 3A to NADH (1 mM) in aqueous suspensions, in accordance with an embodiment of the disclosure;

FIG. 3G graphically illustrates selectivity of DPA-CNPPV Pdots in the presence of various potential interfering biologically relevant analytes (1 mM): (1) water, (2) NADH, (3) NAD⁺, (4) glucose, (5) H₂O₂, (6) lactate, (7) citrate, (8) Na⁺, (9) K⁺, (10) Ca²⁺, (11) Mg²⁺, (12) Cl⁻, in accordance with an embodiment of the disclosure;

FIG. 3H graphically illustrates emission spectra of DPA-CNPPV Pdots in the presence of various potential interfering biologically relevant analytes (1 mM): (1) water, (2) NADH, (3) NAD⁺, (4) glucose, (5) H₂O₂, (6) lactate, (7) citrate, (8) Na⁺, (9) K⁺, (10) Ca²⁺, (11) Mg²⁺, (12) Cl⁻, in accordance with an embodiment of the disclosure;

FIG. 3I graphically illustrates reversibility of the responsiveness of DPA-CNPPV Pdots, in accordance with an embodiment of the disclosure, to NADH (1 mM);

FIG. 3J illustrates emission spectra of DPA-CNPPV Pdots and NADH at 0 and 2 mM NADH with excitation at 385 nm, in accordance with an embodiment of the disclosure;

FIG. 3K is a photograph of Pdot of FIG. 3J in a solution illuminated with UV light at 365 nm, in accordance with an embodiment of the present disclosure;

FIG. 3L graphically illustrates a fluorescence response of the Pdots of FIG. 3J to NADH and NADPH, where a titration of the DPA-CNPPV/PSMA Pdot with NADH, NADPH, NAD⁺, and NADP⁺ showed a fluorescence response only to NADH and NADPH, indicating that NAD⁺ and NADP⁺ do not quench the Pdot emission, and do not themselves emit at 458 nm under UV illumination, in accordance with an embodiment of the disclosure;

FIG. 4A is a merged bright-field and fluorescence microscopy image of PFBT Pdots-labeled MCF-7 cells, in accordance with an embodiment of the disclosure, treated with phosphate-buffered saline (PBS);

FIG. 4B graphically illustrates three-dimensional fluorescence intensity of the image of FIG. 4A, in accordance with an embodiment of the disclosure;

FIG. 4C is a merged bright-field and fluorescence microscopy image of PFBT Pdots-labeled MCF-7 cells, in accordance with an embodiment of the disclosure, treated with NADH;

FIG. 4D graphically illustrates three-dimensional fluorescence intensity of the image of FIG. 4C, in accordance with an embodiment of the disclosure;

FIG. 5A is a photograph of DPA-CNPPV Pdots, in accordance with an embodiment of the disclosure, in solution at physiologically relevant NADH range (0-2 mM) taken under illumination with a UV light at 365 nm;

FIG. 5B shows original region of interest (ROI) images from FIG. 5A split into their RGB channels, in accordance with an embodiment of the disclosure;

FIG. 5C is a ratiometric calibration plot (R/R₀) of the Pdots of FIG. 5A in a physiologically relevant NADH range (0-2 mM), in accordance with an embodiment of the disclosure;

FIG. 5D shows a three-dimensional distribution of the fluorescence intensity of the Pdots of FIG. 5A in the absence of NADH (0 mM), in accordance with an embodiment of the disclosure;

FIG. 5E shows a three-dimensional distribution of the fluorescence intensity of the Pdots of FIG. 5A in the presence of NADH (2 mM), in accordance with an embodiment of the disclosure;

FIG. 5F graphically illustrates a mean of R/R₀ fluorescence intensity ratio of the Pdots of FIG. 5A in the absence and presence of NADH, in accordance with an embodiment of the disclosure;

FIG. 5G schematically illustrates ratiometric imaging of NADH in vivo with DPA-CNPPV Pdots and a smartphone, in accordance with an embodiment of the present disclosure, where emission shifts from red to blue as NADH concentration increases when pdots were injected into mice at two locations, with and without NADH (0.1 mmol), inset: heatmap images of the ratio of blue- and red-channel intensities (B/R ratio) from the two injection regions are shown on the right, where a high B/R ratio (red) indicates high NADH concentration;

FIG. 5H illustrates concentration-dependent, ratiometric imaging of NADH in live mice with a smartphone camera, in accordance with an embodiment of the present disclosure, where regions of interest (square-marked area) correspond to locations of subcutaneous injections of DPA-CNPPV Pdots either alone (0.1 mg/mL, 100 μL; 0 mM NADH) or together with NADH (0.25, 0.5, and 1.0 mM);

FIG. 5I illustrates fluorescence intensities of the B and R channels of FIG. 5G, in accordance with an embodiment of the present disclosure;

FIG. 5J illustrates a mean R/R₀ for the regions of interest of FIG. 5H;

FIG. 6A graphically illustrates fluorescence spectra of the Pdots, in accordance with an embodiment of the disclosure, in the presence of various concentrations of phenylalanine (0-2400 μM);

FIG. 6B is a ratiometric calibration plot (R/R0) of the Pdots of FIG. 6A as a function of phephenylalanine concentration, in accordance with an embodiment of the disclosure;

FIG. 6C is a ratiometric calibration plot (R/R0) of the Pdots of FIG. 6A at a phenylalanine concentration of 0-120 μM, in accordance with an embodiment of the disclosure;

FIG. 6D is a ratiometric calibration plot (R/R0) of the Pdots of FIG. 6A at a phenylalanine concentration of 120-360 μM corresponding to mild benign HPA, in accordance with an embodiment of the disclosure;

FIG. 6E is a ratiometric calibration plot (R/R0) of the Pdots of FIG. 6A at a phenylalanine concentration of 360-600 μM corresponding to mild HPA, in accordance with an embodiment of the disclosure;

FIG. 6F is a ratiometric calibration plot (R/R0) of the Pdots of FIG. 6A at a phenylalanine concentration of 600-900 μM nm corresponding to mild PKU, in accordance with an embodiment of the disclosure;

FIG. 6G is a ratiometric calibration plot (R/R0) of the Pdots of FIG. 6A at a phenylalanine concentration of 900-1200 μM corresponding to moderate PKU, in accordance with an embodiment of the disclosure;

FIG. 6H is a ratiometric calibration plot (R/R0) of the Pdots of FIG. 6A at a phenylalanine concentration of 1200-1800 μM corresponding to classic PKU, in accordance with an embodiment of the disclosure;

FIG. 6I is a ratiometric calibration plot (R/R0) of the Pdots of FIG. 6A at a phenylalanine concentration of 1800-2400 μM corresponding to classic PKU, in accordance with an embodiment of the disclosure;

FIG. 7A is an image of 96-well assay microplate loaded with Pdots, in accordance with an embodiment of the disclosure, in solution with various phenylalanine concentrations (100 μL well⁻¹) with a highlighted portion showing phenylalanine concentrations;

FIG. 7B is an image of a digital camera of a system, in accordance with an embodiment of the present disclosure, configured to image the 96-well assay microplate of FIG. 7A;

FIG. 7C is a ratiometric calibration plot (R/R₀) of the Pdots of FIG. 7B as a function of phenylalanine concentration, in accordance with an embodiment of the disclosure;

FIG. 7D is an image of a smartphone camera of a system of the present disclosure for microplate readout, in accordance with an embodiment of the disclosure;

FIG. 7E is a ratiometric calibration plot (R/R₀) of the Pdots sensor of FIG. 7D as a function of phenylalanine concentration, in accordance with an embodiment of the disclosure;

FIG. 7F is an image of a transducer substrate, in accordance with an embodiment of the disclosure;

FIG. 7G is an image of a fluorescence plate reader, in accordance with an embodiment of the disclosure, for measuring transducer substrates of FIG. 7F;

FIG. 7H is a ratiometric calibration plot (R/R₀) of the Pdots of FIG. 7A as a function of phenylalanine concentration from fluorescence measured from the transducer substrate of FIG. 7F, in accordance with an embodiment of the disclosure;

FIG. 7I schematically illustrates a phenylalanine sensing mechanism, in accordance with an embodiment of the present disclosure, where phenylalanine dehydrogenase catalyzes the oxidation of L-phenylalanine by NAD⁺, resulting in stoichiometric formation of NADH. NADH quenches Pdot fluorescence emission at 627 nm, and also fluoresces at 458 nm. As phenylalanine concentration increases, fluorescence emission shifts from red (Pdot emission) to blue (NADH emission). Metabolite concentration is measured ratiometrically—based on the ratio of blue-to-red channel emission intensities, with a digital camera or plate reader—in solution- or paper-based assay formats;

FIG. 7J illustrates pixel intensity distributions in a single well in blue and red channels at 60 μM Phe (healthy); in accordance with an embodiment of the present disclosure;

FIG. 7K illustrates pixel intensity distributions in a single well in blue and red channels at 1200 μM Phe (classic PKU threshold), in accordance with an embodiment of the present disclosure;

FIG. 7L illustrates a mean ratio of blue- and red-channel emission showing a significant increase between 60 μM and 1200 μM Phe as in FIGS. 7J and 7K;

FIG. 8 shows examples of molecular structures of conjugated polymers, PFO, PDHF, PFBT, PFBTTBT, PFTBT, DPA-CNPPV, and DPA-CNPF, suitable for use in Pdots, in accordance with an embodiment of the present disclosure;

FIG. 9 graphically illustrates hydrodynamic diameters of PFBT Pdots, in accordance with an embodiment of the disclosure, in a PBS solution as a function of storage time at room temperature, where error bars represent the standard deviations of the three measurements;

FIG. 10 graphically illustrates fluorescence emission of PFO Pdots, in accordance with an embodiment of the disclosure, with increasing the NADPH concentration (λₑₓ=380 nm);

FIG. 11 graphically illustrates a ratio F₀/F of fluorescence emission of PFO Pdots, in accordance with an embodiment of the disclosure, in the absence (F₀) and presence (F) of NADH;

FIG. 12A graphically illustrates F₀/F for PDHF Pdots, λₑₘ=428 nm, in accordance with an embodiment of the disclosure, at varying NADH concentrations (0-2 mM);

FIG. 12B graphically illustrates F₀/F for PFBT Pdots, λₑₘ=546 nm, in accordance with an embodiment of the disclosure, at varying NADH concentrations (0-2 mM);

FIG. 12C graphically illustrates F₀/F for PFBTTBT Pdots, λₑₘ=626 nm, in accordance with an embodiment of the disclosure, at varying NADH concentrations (0-2 mM);

FIG. 12D graphically illustrates F₀/F for PFTBT Pdots, λₑₘ=638 nm, in accordance with an embodiment of the disclosure, at varying NADH concentrations (0-2 mM);

FIG. 13 graphically illustrates fluorescence emission of PFBTTBT Pdots, in accordance with an embodiment of the disclosure, before and after adding NADH (10 mM) obtained by exciting at λₑₓ=380 nm;

FIG. 14 graphically illustrates fluorescence of DPA-CNPF Pdots, in accordance with an embodiment of the disclosure, with increasing the NADH concentration (λₑₓ=385 nm);

FIG. 15 is a schematic illustration of PFBT Pdots bioconjugation, in accordance with an embodiment of the present disclosure, for specific cellular targeting;

FIG. 16 is a schematic illustration metabolite quantification by NAD(P)H level, where a specific enzymatic reaction is performed with NAD(P)H-dependent enzymes to oxidize the analyte of interest by NAD(P)⁺ and the level of NAD(P)H corresponds to the level of the analyte in the sample, in accordance with an embodiment of the disclosure;

FIG. 17A graphically illustrates fluorescence spectra of a Pdot sensor, according to an embodiment of the disclosure, with lactic dehydrogenase in the presence of various concentrations of lactate;

FIG. 17B is a ratiometric calibration plot (R/R₀) of the Pdot sensor of FIG. 17A as a function of lactate concentration, in accordance with an embodiment of the disclosure;

FIG. 17C graphically illustrates fluorescence spectra of a Pdot sensor, according to an embodiment of the disclosure, with glutamic dehydrogenase in the presence of various concentrations of glutamate, in accordance with an embodiment of the disclosure;

FIG. 17D is a ratiometric calibration plot (R/R₀) of the Pdot sensor of FIG. 17C as a function of glutamate concentration, in accordance with an embodiment of the disclosure;

FIG. 17E graphically illustrates fluorescence spectra of a Pdot sensor, according to an embodiment of the disclosure, with glucose dehydrogenase in the presence of various concentrations of glucose;

FIG. 17F is a ratiometric calibration plot (R/R₀) of the Pdot sensor of FIG. 17E as a function of glucose concentration, in accordance with an embodiment of the disclosure;

FIG. 17G graphically illustrates fluorescence spectra of a Pdot sensor, according to an embodiment of the disclosure, with β-hydroxybutyrate (BHB) dehydrogenase in the presence of various concentrations of BHB;

FIG. 17H is a ratiometric calibration plot (R/R₀) of the Pdot sensor of FIG. 17G as a function of BHB concentration, in accordance with an embodiment of the disclosure;

FIG. 18A illustrates fluorescence emission of a phenylalanine biosensor, in accordance with an embodiment of the disclosure, in the presence or absence of phenylalanine dehydrogenase (PheDH);

FIG. 18B illustrates background correction for endogenous NADH levels, where the sensor of FIG. 18A without PheDH measures endogenous NADH and subtracting this value from the value with PheDH yields the phenylalanine concentration, in accordance with an embodiment of the present disclosure; and

FIGS. 18C and 18D illustrate fluorescence measured using a fluorescence plate reader (18C) and a digital camera (18D) of phenylalanine spiked into plasma samples containing phenylalanine biosensors, in accordance with an embodiment of the present disclosure.

### DETAILED DESCRIPTION

The present disclosure relates generally to apparatuses, compositions, kits, systems, and methods for monitoring, determining, and/or measuring the concentration of analytes in fluids using transducers. In an embodiment, the analyte is a molecule in the fluid. In an embodiment, the fluid is blood; for example, the compositions, systems, and methods disclosed herein are useful for monitoring the concentration of one or more selected molecules in the blood of a subject. In an embodiment, the fluid is tears; for example, the compositions, systems, and methods disclosed herein are useful for monitoring the concentration of one or more selected molecules in the tear of a subject. In an embodiment, the fluid is sweat; for example, the compositions, systems, and methods disclosed herein are useful for monitoring the concentration of one or more selected molecules in the sweat of a subject. In an embodiment, the fluid is saliva; for example, the compositions, systems, and methods disclosed herein are useful for monitoring the concentration of one or more selected molecules in the saliva of a subject. In an embodiment, the fluid is lymph fluid; for example, the compositions, systems, and methods disclosed herein are useful for monitoring the concentration of one or more selected molecules in the lymph fluids of a subject. In an embodiment, the fluid is spinal fluid; for example, the compositions, systems, and methods disclosed herein are useful for monitoring the concentration of one or more selected molecules in the spinal fluid of a subject. In an embodiment, the fluid is urine; for example, the compositions, systems, and methods disclosed herein are useful for monitoring the concentration of one or more selected molecules in the urine of a subject.

In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Transducers

In an aspect, the present disclosure provides transducers for monitoring a concentration of an analyte in a fluid. As described further herein, in an embodiment, such transducers are suitable to emit a signal, such as an optical signal, that is based on or proportional to a concentration of an analyte adjacent to components of the transducer. In such an embodiment, the transducer is configured to generate an optical signal based upon the presence or absence or concentration of the analyte.

In an embodiment, the transducer is a nanoparticle transducer, such as a nanoparticle transducer for analyte concentration measurements, the nanoparticle transducer comprising a nanoparticle comprising one or more chromophore, such as one or more semiconducting chromophoric polymer; and an enzyme physically associated, such as coupled, to the nanoparticle. In an embodiment, the enzyme is a nicotinamide adenine dinucleotide (NADH)-dependent or nicotinamide adenine dinucleotide phosphate (NADPH)-dependent enzyme. In an embodiment, the activity or stoichiometry (between analyte and one or more reaction element) of an NADH-dependent or NADPH-dependent enzyme, such as the rate at which it acts upon a substrate, depends on concentrations of NADH or NADPH and/or the concentrations of NAD⁺ or NADP⁺ adjacent to the enzyme. In an embodiment, the analyte comprises NADH or NADPH, or NAD⁺ or NADP⁺. As discussed further herein with respect to the EXAMPLES of the present disclosure, it has been surprisingly found that transducers as described herein are suitable to monitor or determine a concentration of an analyte and/or reaction element including NADH and/or NADPH or NAD⁺ and/or NADP⁺.

In an embodiment, the enzyme is configured to catalyze a reaction comprising a plurality of reaction elements. In an embodiment, the plurality of reaction elements comprises one or more reactants including the analyte and one or more products, and wherein an amount of fluorescence emitted from the chromophore is determined by and/or based on a concentration of a reaction element of the plurality of reaction elements. In an embodiment, a reaction element of the plurality of reaction elements comprises NADH and wherein the amount of fluorescence emitted from the chromophore is determined by a concentration of the NADH.

In another embodiment, the transducer is a transducer substrate for analyte concentration measurements, the transducer substrate comprises: a nanoparticle comprising a chromophore coupled to a substrate; and an enzyme coupled to the substrate and configured to catalyze a reaction comprising a plurality of reaction elements. As discussed further herein with respect to EXAMPLE 7, it has been surprisingly found that transducers including a chromophore, such a nanoparticle, and an enzyme coupled to a common substrate are suitable to measure and/or monitor an analyte concentration.

As above, in an embodiment of the transducer substrate, the nanoparticle and the enzyme are coupled to the substrate. The substrate can include any substrate suitable for coupling to a nanoparticle and an enzyme. Such substrates can include, without limitation, polymeric substrates, glass substrates, silica-based substrates, silicon-based substrates, metal substrates, woven substrates, non-woven substrates, and the like. In an embodiment, the substrate is a fibrous and/or paper-based substrate. In an embodiment, the substrate is a porous membrane. In an embodiment, the porous membrane includes paper, nitrocellulose, nylon, and many other materials recognized by those skilled in the art as capable of serving as a wick. In an embodiment, the substrate is a paper substrate.

In an embodiment, the enzyme is covalently bonded to the substrate. In an embodiment, the nanoparticle is covalently bonded to the substrate. In an embodiment, the enzyme and/or the nanoparticle is/are physically associated with the substrate. Such physical association can include covalent bonding, but can also include other non-covalent associations between the enzyme, nanoparticle, and substrate. In this regard, the enzyme and/or nanoparticle can be associated with the substrate through ionic bonding, van der Waals forces, hydrogen bonding, and the like. In an embodiment, the enzyme and/or nanoparticle are deposited on the substrate, such as through liquid deposition or blotting. In an embodiment, the enzyme and the nanoparticle are lyophilized onto the substrate.

In an embodiment, the nanoparticle is coupled to the substrate in a spot or other spatially limited area of the substrate, such as a spot or area that comprises less than all of the substrate. Such limited placement or localization of the nanoparticle on the substrate allows for various sample and reaction conditions to be tested and analyzed as discussed further herein. In an embodiment, the enzyme is coupled to the substrate adjacent to the nanoparticle on a spot. In an embodiment, the enzyme is coupled to the substrate and the nanoparticle is coupled to the substrate on a spot.

In an embodiment, the substrate includes a number of nanoparticles coupled to the substrate in a spatially distinct portions or spot. In an embodiment, the substrate includes a number of enzymes also coupled to the spots and corresponding to the number of nanoparticles also coupled thereto. In this regard, in an embodiment, the substrate includes pairs of nanoparticles and enzymes or types of nanoparticles and types of enzymes coupled to the substrate at various spots.

In an embodiment, the substrate further includes one or more nanoparticles coupled to a region or spot on the substrate that does not include an enzyme coupled thereto. Such one or more nanoparticles coupled to the substrate without an enzyme are suitable for use as a control, such as a control spot for calibration.

In an embodiment, the enzyme is a first enzyme, the nanoparticle is a first nanoparticle comprising a first chromophore, and the reaction is a first reaction. In such an embodiment, the transducer substrate can further comprise a second nanoparticle comprising a second chromophore coupled to the substrate; and a second enzyme different from the first enzyme coupled to the substrate configured to catalyze a second reaction comprising a second plurality of reaction elements. In an embodiment, the second plurality of reaction elements comprise one or more second reactants including the analyte and one or more second products, and wherein an amount of fluorescence emitted from the second chromophore is determined by a concentration of a second reaction element of the second plurality of reaction elements.

In an embodiment, the spot is a first spot, and wherein the second nanoparticle is coupled to the substrate on a second spot separate from the first spot. In this regard, a sample may be applied to the first and the second spot in order to, for example, assay the sample with different enzymes, such as where the first and second enzyme are different. In an embodiment, the second reaction is different from the first reaction. In an embodiment, a single sample is applied to different spots for the determination of the concentrations of different analytes by the different spots using spatial multiplexing. In an embodiment, the sample may be applied to the first and the second spot in order to perform multiple iterations of the same reaction, such as where the first and the second enzyme are the same. Accordingly, in an embodiment, the second reaction is the same as the first reaction. In an embodiment, the sample is applied to the first and the second spot in order to perform the same reaction, but over a different concentration range or dynamic range, in which case the first and the second enzyme are also the same. Accordingly, in an embodiment, the second reaction is the same as the first reaction.

In an embodiment, the second nanoparticle is coupled adjacent to the first nanoparticle on the substrate. Accordingly, in an embodiment, the second nanoparticle is coupled to the substrate on the spot. Such embodiments may be suitable for assaying a single sample with nanoparticles including optically distinct chromophores for determining concentrations of different analytes by the different nanoparticles with distinct chromophores using spectral multiplexing.

In an embodiment, the first chromophore is configured to absorb light in a first absorption wavelength range and the second chromophore is configured to absorb light in a second absorption wavelength range different from the first absorption wavelength range. Such a configuration is suitable for, for example, absorption or excitation multiplexing in which different nanoparticles are excited with different wavelengths of light and/or different light sources. Likewise, in an embodiment, the fluorescence emitted from the first chromophore is in a first emission wavelength range, and wherein fluorescence emitted from the second chromophore is in a second emission wavelength range different from the first emission wavelength range. Such a configuration is suitable for emission multiplexing in which fluorescence from different nanoparticles is detected in different wavelength ranges and/or with different sensors. In an embodiment, the first chromophore and second chromophore are configured to absorb light at the same or similar absorption wavelength range but emit light in different wavelength ranges. Such a configuration utilizes emission multiplexing but not excitation multiplexing. In an embodiment, the first chromophore and second chromophore are configured to emit light at the same or similar emission wavelength range but absorb light in different wavelength ranges. Such a configuration utilizes excitation multiplexing but not emission multiplexing. In an embodiment, the first chromophore and second chromophore are configured to absorb light in different absorption wavelength range and emit light in different emission wavelength range. Such a configuration utilizes both excitation and emission multiplexing.

As above, in certain embodiments, nanoparticles and enzymes are coupled to the substrate in a number of spatially separate portions or spots, such as for multiplexing different reactions, performing the same reaction under different concentrations, or performing duplicates of a reaction. In an embodiment, a number of spots on the substrate containing a nanoparticle and an enzyme coupled thereto is selected from 2, 4, 6, 8, 24, 96, 384, and 1536. Such spots may be configured to correspond spatially to wells of a standard multi-well plate and, accordingly, readers or other sensors used to measure, for example, fluorescence of samples contained in wells of such standard multi-well plates. In an embodiment, a number of spots on the substrate containing a nanoparticle and an enzyme coupled thereto is in a range of 2 to 10, 2 to 50, 2 to 100, 2 to 500, or 2 to 1,000, or more.

Enzymes and nanoparticles may be applied to substrates, such as paper-based or other porous substrates, to have high spatial resolution and small spot size. Such a configuration is suitable for assaying large numbers of different nanoparticle/enzyme pairs on a single substrate. In an embodiment, a size of the spot is in a range of about 1 μm to about 10 μm, about 1 μm to about 25 μm, about 1 μm to about 50 μm, about 1 μm to about 100 μm, about 1 μm to about 250 μm, about 1 μm to about 400 μm, or about 1 μm to about 500 μm.

In an embodiment, the substrate is configured to wick a fluid sample to the spot. Such a wicking substrate is suitable for reactions and assays in which a fluid sample is applied to a first portion of the substrate and wicked, such as through capillary action, to another portion of the substrate, such as to which the nanoparticle(s) and enzyme(s) are coupled. In an embodiment, the substrate includes one or more fluidically isolated paths leading from an application zone and separately to spatially distinct spots on which pairs of enzymes and nanoparticles are deposited.

In an embodiment, the wicking substrate is configured to filter or separate out cells or other particulates that may interfere with the measurement, such as blood cells, from a fluid sample such that fluid arriving at one or more spots on the substrate is free of or substantially free of cells or other particulates that may interfere with the measurement. In an embodiment, the substrate includes a sample application portion and a filter disposed between the sample application portion and the one or more spots in fluidic communication with the sample application portion.

In an embodiment, the nanoparticle comprises a polymer dot (Pdot). As used herein, the term “polymer dot” or “Pdot” refers to a particle structure including one or more semiconducting polymers collapsed to form a stable sub-micron sized particle, e.g., a nanoparticle. In an embodiment, the polymer dots are highly fluorescent nanoparticles with emissions tunable, e.g., from the visible to the near IR region. The polymer dots can include chromophoric polymers that can, e.g., absorb light and then emit light by fluorescence. In some embodiments, the polymer dots include at least one condensed polymer, e.g., a semiconducting polymer. For polymer dots having more than one condensed polymer (e.g., more than one semiconducting polymer), the condensed polymers can be the same or different types of polymers. For example, a Pdot can comprise both semiconducting polymers and non-semiconducting polymers.

A nanoparticle transducer to monitor a selected analyte may be assembled from an appropriate choice of an enzyme, a nanoparticle, and a chromophore. As discussed further herein, the enzyme need not be coupled, such as covalently bound, to the nanoparticle. The enzyme can be chosen as an enzyme that catalyzes a reaction involving the analyte, such that the concentration of the analyte can affect the rate of the reaction or the amount of reaction elements produced or consumed. The reaction can involve a plurality of reaction elements, including reactants and products. The enzyme can be selected such that each reactant of its catalyzed reaction is present in the fluid to be analyzed. The chromophore can be chosen such that fluorescence of the chromophore is determined by the concentration of a reactant or product of the reaction catalyzed by the enzyme or by the rate at which the reactant is consumed or product formed. The nanoparticle can be chosen to allow both the enzyme and the chromophore to be incorporated into or conjugated with the nanoparticle. For example, the nanoparticle can be a Pdot, allowing the enzyme to be covalently bonded to the Pdot and the chromophore to be incorporated into and/or covalently bonded to the Pdot. In some cases, the chromophore can comprise all or substantially all of the nanoparticle; for example, a Pdot may be made entirely or substantially entirely of one or more chromophores, in some cases.

While transducers including nanoparticles, such as Pdots are described, in certain embodiments, the transducers of the present disclosure include a chromophore in an uncondensed state. For example, in an embodiment, the transducer includes a chromophore including a semiconducting chromophoric polymer; and an enzyme physically associated with the semiconducting chromophoric polymer and configured to catalyze a reaction comprising a plurality of reaction elements, wherein such a chromophore does not include a condensed semiconducting chromophoric polymer.

In an embodiment, the uncondensed semiconducting chromophoric polymer and the enzyme are coupled to a substrate. In an embodiment, the uncondensed semiconducting chromophoric polymer and the enzyme are in a lyophilized powder. In an embodiment, the semiconducting chromophoric polymer and the enzyme are dispersed in a common solvent.

In an embodiment, the enzyme, chromophore and nanoparticle can be selected from a set of potential enzymes, chromophores, and nanoparticles to create a nanoparticle transducer to detect a given analyte as follows: From a set of enzymes, select those that catalyze a reaction wherein the analyte is a reactant. For each such reaction, identify the other reaction elements whose concentrations will change as a result of the reaction taking place—for example, each time the reaction takes place, reactant concentrations fall and product concentrations rise (for a reversible reaction, the reverse of the reaction causes the opposite effect). From those reaction elements, for each enzyme, identify a corresponding chromophore from the set of chromophores that has its amount of fluorescence change in response to changes in concentration of one of the reaction elements. If no chromophore matches, eliminate the enzyme. From those enzyme/chromophore pairs remaining, select one such pair and choose a nanoparticle, such as a Pdot, to which each can be physically associated or coupled and/or incorporated, thereby choosing elements to construct a nanoparticle transducer. A second chromophore that emits at a different wavelength and does not change its intensity in response to any reaction elements can be selected from the list of chromophores to serve as a control chromophore. Alternatively, if the originally selected chromophore emits fluorescence both at a wavelength that changes intensity in response to reactant or product concentration and at a different wavelength that does not change intensity, then that single chromophore can serve as its own control.

In an embodiment, the transducers described herein comprise an enzyme catalyzing a reaction involving an analyte. The reaction has reaction elements including reactants and products, one of which is the analyte. The nanoparticle comprises a chromophore that emits fluorescence at one or more wavelengths in response to illumination with a light beam. The amount of fluorescence of at least one of the wavelengths depends on the concentration of a molecule of the reactants or products other than the analyte. The enzyme and the chromophore of the nanoparticle are in proximity or are physically associated; accordingly, as the reaction catalyzed by the enzyme consumes reactants and produces products, the respective concentrations of said reactants and products changes, with reactant concentrations decreasing and product concentrations increasing. The presence of the analyte at elevated concentration causes the reaction to proceed more quickly than at low concentration, so the presence of the analyte results in relatively high product concentrations and relatively low reactant concentrations. Accordingly, the enzyme and the chromophore of the nanoparticle, together, act as a transducer, transforming variations in analyte concentration to variations in fluorescence. In an embodiment, the fluorescence intensity of one wavelength emission of the transducer is used to determine the analyte concentrations. In an embodiment, the fluorescence intensity ratio at two wavelength emissions of the transducer is used to determine the analyte concentrations. This fluorescence can easily be measured in a wavelength-selective manner to determine the analyte's concentration from a signal of an optical sensor.

In an embodiment, the nanoparticle comprises a semiconducting polymer that emits fluorescence at one or more wavelengths in response to illumination with a light beam. The amount of fluorescence at least one of the wavelengths depends on the concentration of a molecule of the reactants or products other than the analyte. In some cases, the nanoparticle comprises a semiconducting polymer and a dye that emits fluorescence at one or more wavelengths. The dye can be physically doped or chemically attached to the semiconducting polymer to form nanoparticles. The semiconducting polymer can have energy transfer to the dye to enhance or amplify the fluorescence intensity of the dye.

In an embodiment, the fluid described herein is a bodily fluid, such as a bodily fluid within or removed from the body of a subject, such as blood, plasma, serum, sweat, tears, lymph fluid, spinal fluid, urine, saliva, or other fluids within or from body tissues or secreted by body tissues. The subject can be an animal, and, in an embodiment, the subject is a human.

Various embodiments of the present disclosure provide chromophores having characteristics that are advantageous for efficient and accurate measurement of analyte concentrations using the transducers provided herein. Examples of such characteristics include but are not limited to: (1) high brightness so the transducer signal can be easily detected and recovered; (2) high sensitivity to a reaction element of the reaction catalyzed by the enzyme; (3) high absorption cross-section so the nanoparticle transducer fluorescence can be easily induced without requiring intense energy application; (4) good stability (e.g. thermostability) so the transducers can remain active for long time periods in vivo; (5) wavelengths capable of being detected and differentiated, including transdermally in some cases; and/or (6) good fatigue resistance for to decrease degradation when used for continuous analyte monitoring. In an embodiment, the chromophores of the nanoparticle transducers described in the present disclosure include some or all of these characteristics.

For instance, the present disclosure provides, in an embodiment, transducers exhibiting signal fluorescent emission intensity at a peak emission wavelength that varies as a function of the concentration of a fluid constituent. The nanoparticle transducer can also comprise a chromophore with a different, control emission intensity at the peak emission wavelength that does not substantially vary in response to the concentration of the fluid constituent. In an embodiment, the peak emission wavelength is within a range from about 200 nanometers to about 300 nanometers, about 250 nanometers to about 350 nanometers, about 300 nanometers to about 400 nanometers, about 350 nanometers to about 450 nanometers, about 400 nanometers to about 500 nanometers, about 450 nanometers to about 550 nanometers, about 500 nanometers to about 600 nanometers, about 550 nanometers to about 650 nanometers, about 600 nanometers to about 700 nanometers, about 650 nanometers to about 750 nanometers, about 700 nanometers to about 800 nanometers, about 750 nanometers to about 850 nanometers, about 800 nanometers to about 900 nanometers, about 850 nanometers to about 950 nanometers, about 900 nanometers to about 1000 nanometers, about 950 nanometers to about 1050 nanometers, about 1000 nanometers to about 1100 nanometers, about 1150 nanometers to about 1250 nanometers, or about 1200 nanometers to about 1300 nanometers.

As another example, some embodiments of the present disclosure provide transducers exhibiting sufficient stability for long-term in vivo analyte concentration monitoring, e.g., the transducers are capable of stably detecting analyte concentration for an extended period of time without substantial degradation. In various embodiments, stability of the nanoparticle transducers is advantageous in ensuring that said transducers can be used in vivo for long time periods without need for replacement. In an embodiment, a population of transducers is considered to be “stable” if at least 50%, at least 60%, at least 70%, at least 75%, at least 80%, at least 85%, at least 90%, at least 95%, at least 99%, at least 99.5% or at least 99.95% of the transducers in the population retain the ability to modulate fluorescence in response to analyte concentration variation for the specified time period. In an embodiment, a transducer is considered to be “stable” if the emission intensity of the transducer retains the ability to measure analyte concentration variation for the specified time period. In an embodiment, a transducer is considered to be “stable” if the intensity ratio of two emission peaks retain the ability to measure analyte concentration variation for the specified time period, even though the absolute emission intensity can be significantly decreased. In an embodiment, a transducer is considered to be stable if the time constant (e.g., time to decay to 1/e of the fluorescence signal strength) is at least about 3 hours, about 6 hours, about 12 hours, about 24 hours, about 1 day, about 2 days, about 4 days, about 10 days, about 20 days, about 30 days, about 1 month, about 2 months, about 4 months, about 6 months, about 1 year, or more. In an embodiment, the nanoparticle transducers maintain sufficient signal intensity that analyte detection can be reliably performed throughout the specified time period.

In an embodiment of the present disclosure, the chromophore emission spectrum is selected or designed to exhibit narrow band emission properties at the peak emission wavelength so as to reduce or minimize overlap with other emission sources. For example, In an embodiment, the chromophore has a peak emission bandwidth (e.g., full width at half maximum (FWHM) of the emission peak) of no more than about 5 nanometers, about 10 nanometers, about 15 nanometers, about 20 nanometers, about 25 nanometers, about 30 nanometers, about 35 nanometers, about 40 nanometers, about 45 nanometers, about 50 nanometers, about 60 nanometers, about 70 nanometers, about 80 nanometers, about 90 nanometers, or about 100 nanometers.

Chromophore Compositions

Various types of chromophores are suitable for use with the transducer, compositions, methods, kits, and systems of the present disclosure, including but not limited to dyes, stains, proteins, polymers, beads, particles, or combinations thereof. In an embodiment, the transducer includes one or more chromophores (e.g., fluorophores). The chromophores described herein can be used to produce transducers according to various mechanisms. In an embodiment, the chromophore comprises a semiconducting polymer that emits fluorescence at one or more wavelengths in response to illumination with a light beam. In an embodiment, the chromophore comprises a semiconducting polymer Pdot that emits fluorescence at one or more wavelengths in response to illumination with a light beam. The amount of fluorescence of the semiconducting polymer can depend on the concentration of a molecule of the reactants or products. The amount of fluorescence of the semiconducting polymer at one wavelength range can depend on the concentration of a molecule of the reactants or products while the amount of fluorescence of the semiconducting polymer at another different wavelength range can be relatively independent of the concentration of a molecule of the reactants or products, and thus can serve as a control wavelength range for ratiometric measurements.

In an embodiment, the transducers of the present disclosure include one or more, such as two or more, chromophores. In an embodiment, the one or more chromophores are configured to emit fluorescence in two or more distinct wavelength ranges, such as is suitable for ratiometric fluorescence measurement as discussed further herein. In an embodiment, the chromophore comprises a semiconducting polymer and a dye that emits fluorescence at one or more wavelengths. The amount of fluorescence of the dye depends on the concentration of a molecule of the reactants or products. The dye can be physically doped or chemically attached to the semiconducting polymer for forming nanoparticles. The chromophoric polymer can have energy transfer to the dye to enhance or amplify the fluorescence intensity of the dye.

In an embodiment, the fluorescence emitted from the chromophore comprises a signal fluorescence wavelength and a control fluorescence wavelength. In an embodiment, the fluorescence emitted from the chromophore defines a signal fluorescence ratio equal to a ratio of an amount of fluorescence emitted at the signal wavelength to an amount of fluorescence emitted at the control fluorescence wavelength, and wherein the signal fluorescence ratio is determined by the concentration of the reaction element of the plurality of reaction elements. In an embodiment, the fluorescence ratio varies ratiometrically with the concentration of the analyte. In an embodiment, fluorescence emitted from the chromophore varies ratiometrically with the concentration of the analyte within a range of analyte concentrations. In an embodiment, fluorescence at the control wavelength stays constant with varying concentrations of the reaction element. In an embodiment, the fluorescence at the control wavelength varies with varying concentrations of the reaction element.

As above, in an embodiment, the chromophore emits fluorescence at two or more distinct wavelength ranges. In an embodiment, the chromophore emits fluorescence at one of the two or more wavelength ranges, such as a control fluorescence wavelength, that remains constant or relatively constant with changing reaction element concentrations and emits fluorescence at another of the two or more wavelength ranges, such as a control fluorescence wavelength, that varies with changing reaction element concentrations. In an embodiment, the chromophore is configured to emit fluorescence at two or more distinct wavelength ranges in which the emitted fluorescence varies with reaction element concentration at least two of the two or more distinct wavelength ranges.

In an embodiment, a transducer includes at least one chromophoric, semiconducting polymer particle (also known as “polymer dot” or “Pdot”) comprising one or more polymers (e.g., semiconducting polymers, non-semiconducting polymers, or a combination thereof) that have been collapsed into a stable sub-micron-sized particle. Semiconducting polymer particles are advantageous in an embodiment compared to other types of chromophores for several reasons: (1) they are extremely bright, up to 30 times brighter than quantum dots, and photostable; (2) they have fast photon emission rates, often with sub-nanosecond lifetimes so they are well-suited for fast optical detection; (3) they possess good biocompatibility and are not composed of cytotoxic heavy metals like quantum dots; and (4) they exhibit amplified energy transfer so their fluorescence emission can be well-modulated, e.g., by dyes via energy transfer.

Various structures and compositions of chromophoric polymer particles are applicable to the aspects presented herein. The chromophoric polymer particles provided herein are made up of a single polymer or, alternatively, comprise blends of polymers. In an embodiment, the one or more polymers are collapsed, precipitated, and/or condensed to form a polymer matrix. In an embodiment, the properties of the chromophoric polymer particle are dependent on the structure and/or properties of the constituent polymer(s). Therefore, the polymer backbone (main chain), side chains, terminal units, and substituted groups are varied, in an embodiment, to obtain specific properties. In an embodiment, the optical properties of the chromophoric polymer particle are tuned by varying the structures of the polymer backbone (main chain).

In an embodiment, the chromophoric polymer particles provided herein include one or more chromophores, also referred to herein as chromophoric units. In an embodiment, a chromophore absorbs certain wavelengths of light, e.g., from the UV region to the near infrared region, and may be or may not be emissive. In an embodiment, a chromophoric unit includes, but is not limited to, a unit of structures with delocalized pi-electrons, a unit of small organic dye molecules, and/or a unit of metal complexes. In various embodiments, the chromophore is part of the polymer matrix or is incorporated into the polymer matrix, e.g., by blending, crosslinking, and the like. In an embodiment, the chromophoric polymer is a semiconducting polymer.

In an embodiment, the chromophoric polymer particles of the present disclosure include one or more chromophoric polymers. In an embodiment, a chromophoric polymer includes at least a portion which absorbs certain wavelengths of light, e.g., ranging from UV to near infrared spectra. Chromophoric polymers according to the present disclosure may be or may not be emissive. In an embodiment, a chromophoric polymer includes one or more chromophoric units. Examples of chromophoric polymers include but are not limited to polymers comprising units of structures with delocalized pi-electrons (e.g., semiconducting polymers), polymers comprising units of small organic dye molecules, polymers comprising units of metal complexes, and polymers comprising units of any combinations thereof. In an embodiment, the chromophoric unit is incorporated into the polymer backbone. In an embodiment, the chromophoric unit is covalently attached to the side chain, or the terminal unit of the polymer. Chromophoric polymers are made using standard synthesis methods generally well known in the art, In an embodiment.

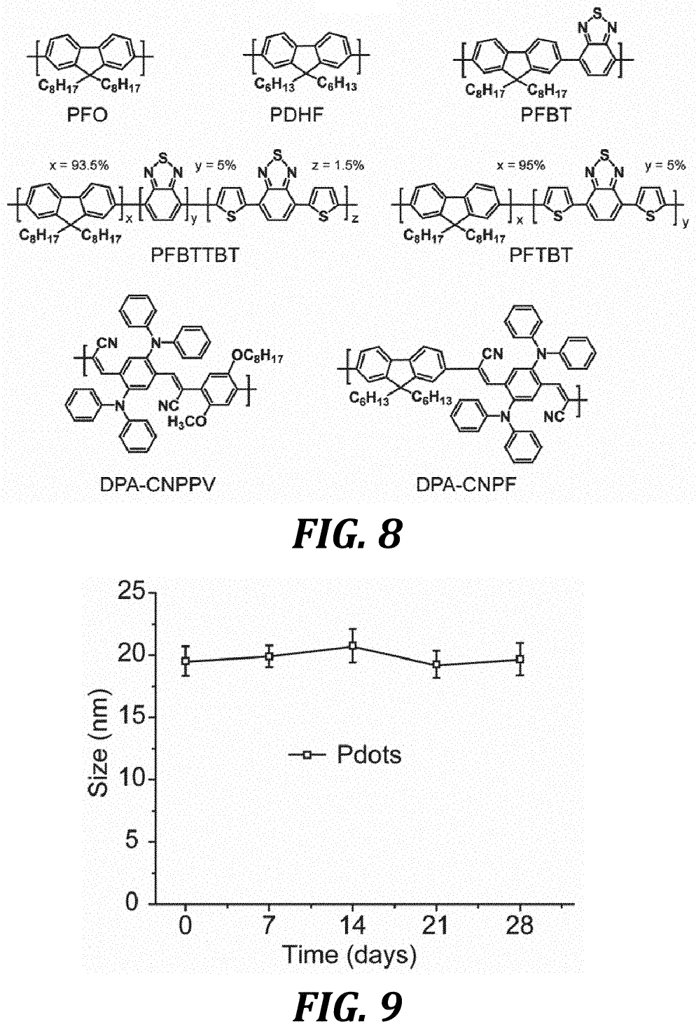

Various types of chromophoric polymer particles are suitable for use as a platform for the optical sensing approaches of the present disclosure. Chromophoric polymer particles can adopt a variety of configurations, including but not limited to a monolithic polymer particle having a uniform, homogenous composition or a polymer particle having a distinct core and cap structure. The chromophoric polymer particles provided herein can be formed by any method known in the art, including, without limitation, methods relying on precipitation, methods relying on the formation of emulsions (e.g., mini or micro emulsion), and methods relying on condensation. Examples of chemical structures of repeating units suitable for the chromophoric polymer particles are illustrated in FIG. 8. Examples of chromophoric polymer particles suitable for use with the techniques described herein can be found in, for example, PCT application numbers PCT/US2010/056079, PCT/US2012/071767, PCT/US2011/056768, PCT/US2013/024300, and PCT/US2013/063917 and in U.S. Patent Publication No. 2013/0266957, each of which is incorporated herein by reference.

In an embodiment, the chromophoric polymer particle is a nanoparticle. In an embodiment, the sizes of the nanoparticles provided herein are defined in terms of a “critical dimension,” which refers to the smallest dimension of the nanoparticle. Some nanoparticles are roughly spherical in shape, which results in the critical dimension being the diameter of the spherical particle. In an embodiment, certain nanoparticles, such as nanospheres and nanocubes, are completely nanoscopic in size. In an embodiment, not every dimension of a nanoparticle is at the nanoscale. For example, a nanocylinder can have a diameter on the nano-scale but a length on the micro-scale. A wide variety of nanoparticle shapes are applicable to the aspects described herein, including but not limited to a sphere, a cylinder, an ellipsoid, a polyhedron, a prism, a rod, a wire, or combinations thereof. The shape of the nanoparticle contributes to the optical properties, in an embodiment, as will be appreciated by those of skill in the art (e.g., nano-rods may have different optical properties than nano-spheres).

In an embodiment, the typical size of a chromophoric polymer particle is fewer than 100 nanometers. In an embodiment, a colloidal polymer nanoparticle is composed of a hydrophobic polymer interior. In an embodiment, the chromophoric polymer particle comprises at least one chromophoric polymer that has been formed into a stable particle. The particle size can vary from 5 nanometers to 500 nanometers, for example. In an embodiment, the critical dimension (e.g. diameter) of the particle is less than 1,000 nanometers, less than 700 nanometers, less than 500 nanometers, less than 400 nanometers, less than 300 nanometers, less than 200 nanometers, less than 100 nanometers, less than 50 nanometers, less than 40 nanometers. In an embodiment, the critical dimension of the particle is less than 30 nanometers, less than 20 nanometers, or less than 10 nanometers.

In an embodiment, the chromophoric polymer particles described herein include a polymer matrix formed from one or more chromophoric polymers. Any suitable number and combination of chromophoric polymer types can be incorporated in the chromophoric polymer particles described herein, such as one or more chromophoric polymers, two or more chromophoric polymers, three or more chromophoric polymers, four or more chromophoric polymers, five or more chromophoric polymers, six or more chromophoric polymers, seven or more chromophoric polymers, eight or more chromophoric polymers, nine or more chromophoric polymers, ten or more chromophoric polymers, fifty or more chromophoric polymers, or one hundred or more chromophoric polymers. The mass concentration or mass ratio of the chromophoric polymers relative to the entire chromophoric polymer particle mass can be varied from 1% to 99%, 10% to 99%, 20% to 99%, 30% to 99%, 40% to 99%, or 50% to 99%.

Various types and compositions of chromophoric polymers are applicable for use in accordance with aspects of the present disclosure. The chromophoric polymer can be a homopolymer or a heteropolymer. In various embodiments, the chromophoric polymer is a semiconducting polymer, a non-semiconducting polymer, or a combination thereof. For example, a number of semiconducting polymers are suitable for use in chromophoric polymer particles according to the present disclosure. Examples of semiconducting polymers include but are not limited to: polyfluorene-based polymers, including but not limited to poly(9,9-dihexylfluorenyl-2,7-diyl) (PDHF)-based and poly(9,9-dioctylfluorenyl-2,7-diyl) (PFO)-based; fluorene-based copolymers, including but not limited to, poly[{9,9-dioctyl-2,7-divinylene-fluorenylene}-alt-co-{2-methoxy-5-(2-ethylhexyloxy)-1,4-phenylene}] (PFPV)-based, poly[(9,9-dioctylfluorenyl-2,7-diyl)-co-(1,4-benzo-{2, 1,3}-thiadiazole)] (PFBT)-based, poly[(9,9-dioctylfluorenyl-2,7-diyl)-co-(4,7-Di-2-thienyl-2,1,3-benzothiadiazole)] (PFTBT)-based, and poly[(9,9-dioctylfluorenyl-2,7-diyl)-co-(4,7-Di-2-thienyl-2,1,3-benzothiadiazole)] (PF-0.1TBT)-based; phenylene vinylene polymers, including but not limited to, poly[2-methoxy-5-(2-ethylhexyloxy)-1,4-phenylenevinylene] (MEH-PPV)-based and poly [2-methoxy-5-(2-ethylhexyloxy)-1,4-(1-cyanovinylene-1,4-phenylene)] (CN-PPV)-based semiconducting polymers; phenylene ethynylene-based polymers, including but not limited to, poly(2,5-di(3′,7′-dimethyloctyl)phenylene-1,4-ethynylene (PPE)-based semiconducting polymers; BODIPY based semiconducting polymer; squaraine based semiconducting polymer; or a combination thereof.

A wide variety of chromophoric polymer structures are suitable for use in accordance with various embodiments of the present disclosure. In an embodiment, the chromophoric polymer is a linear polymer. In other aspects, the chromophoric polymer is a branched polymer. In an embodiment, the chromophoric polymer is a dendrimer. In an embodiment, the chromophoric polymer is a brush polymer. In an embodiment, the chromophoric polymer is a star polymer.

In an embodiment, the chromophoric polymer particles described herein contain a polystyrene-based, comb-like polymer. Non-limiting examples of polystyrene based comb-like polymers include polystyrene graft acrylic acid, polystyrene graft ethylene oxide, polystyrene graft butyl alcohol, and the like. In an embodiment, chromophoric polymer particles described herein contain poly(methyl methacrylate) based comb-like polymers. Non-limiting examples of poly(methyl methacrylate) based comb-like polymers include poly(methyl methacrylate) graft acrylic acid, poly(methyl methacrylate) graft ethylene oxide, and the like. In an embodiment, chromophoric polymer particles described herein contain a comb-like polymer comprising carboxyl, amine, thiol, ester, succinimidyl ester, azide, alkyne, cyclooctyne, or phosphine groups.

In an embodiment, the chromophoric polymer particles described herein contain a polymer functionalized on the terminal repeating unit, for example with a carboxyl, amine, thiol, ester, succinimidyl ester, azide, alkyne, cyclooctyne, phosphine, or similar functional group. Examples of such polymers include but are not limited to poly(meth)acrylate polymers, polyacrylamide polymers, polyisobutylene, polydiene, polyphenylene, polyethylene, poly(ethylene glycol), polylactide, polystyrene, polysiloxane, poly(vinyl pyridine), poly(vinylpyrrolidone), polyurethane, a block copolymer thereof, a random or alternating copolymer thereof, and the like.

In an embodiment, the chromophoric polymer particles described herein contain a copolymer having one or more functionalized repeating units, for example an amphiphilic polymer, including but not limited to: poly((meth)acrylic acid)-based copolymers such as: poly(acrylic acid-b-acrylamide), poly(acrylic acid-b-methyl methacrylate), poly(acrylic acid-b-N-isopropylacrylamide), poly(n-butylacrylate-b-acrylic acid), poly(sodium acrylate-b-methyl methacrylate), poly(methacrylic acid-b-neopentyl methacrylate), poly(methyl methacrylate-b-acrylic acid), poly(methyl methacrylate-b-methacrylic acid), poly(methyl methacrylate-b-N,N-dimethyl acrylamide), poly(methyl methacrylate-b-sodium acrylate), poly(methyl methacrylate-b-sodium methacrylate), poly(neopentyl methacrylate-b-methacrylic acid), poly(t-butyl methacrylate-b-ethylene oxide), poly(2-acrylamido-2-methylpropanesulfonic acid-b-acrylic acid); polydiene-based copolymers such as: poly(butadiene(1,2 addition)-b-ethylene oxide), poly(butadiene(1,2 addition)-b-methylacrylic acid, poly(butadiene(1,4 addition)-b-acrylic acid), poly(butadiene(1,4 addition)-b-ethylene oxide, poly(butadiene(1,4 addition)-b-sodium acrylate), poly(butadiene(1,4 addition)-b-N-methyl 4-vinyl pyridinium iodide), poly(isoprene-b-ethylene oxide), poly(isoprene-b-ethylene oxide), and poly(isoprene-b-N-methyl 2-vinyl pyridinium iodide); poly(ethylene oxide)-based copolymers such as: poly(ethylene oxide-b-acrylic acid), poly(ethylene oxide-b-acrylamide), poly(ethylene oxide-b-butylene oxide), poly(ethylene oxide-b-c-capro lactone), poly(ethylene oxide-b-lactide), poly(ethylene oxide-b-methacrylic acid), poly(ethylene oxide-b-methyl acrylate), poly(ethylene oxide-b-N-isopropylacrylamide), poly(ethylene oxide-b-methyl methacrylate), poly(ethylene oxide-b-nitrobenzyl methacrylate), poly(ethylene oxide-b-N,N-dimethylaminoethylmethacrylate), poly(ethylene oxide-b-propylene oxide), poly(ethylene oxide-b-t-butyl acrylate), poly(ethylene oxide-b-t-butyl methacrylate), poly(ethylene oxide-b-tetrahydrofurfuryl methacrylate), poly(ethylene oxide-b-2-ethyl oxazoline), poly(ethylene oxide-b-2-hydroxyethyl methacrylate), poly(ethylene oxide-b-2-methyl oxazoline); polyisobutylene-based copolymers such as poly(isobutylene-b-acrylic acid), poly(isobutylene-b-ethylene oxide), poly(isobutylene-b-methacrylic acid); polystyrene-based copolymers such as poly(styrene-b-acrylamide), poly(styrene-b-acrylic acid), poly(styrene-b-cesium acrylate), poly(styrene-b-ethylene oxide), poly(styrene-b-ethylene oxide) acid cleavable at the block junction, poly(styrene-b-methacrylic acid), poly(4-styrenesulfonic acid-b-ethylene oxide), poly(styrenesulfonic acid-b-methylbutylene), poly(styrene-b-N,N-dimethylacrylamide), poly(styrene-b-N-isopropyl acrylamide), poly(styrene-b-N-methyl 2-vinyl pyridinium iodide), poly(styrene-b-N-methyl-4-vinyl pyridinium iodide), poly(styrene-b-propylacrylic acid), poly(styrene-b-sodium acrylate) poly(styrene-b-sodium methacrylate), polyp-chloromethyl styrene-b-acrylamide), poly(styrene-co-p-chloromethyl styrene-b-acrylamide), poly(styrene-co-p-chloro methyl styrene-b-acrylic acid), poly(styrene-b-methylbutylene-co-isoprene sulfonate); polysiloxane-based copolymers such as poly(dimethylsiloxane-b-acrylic acid), poly(dimethylsiloxane-b-ethylene oxide), poly(dimethylsiloxane-b-methacrylic acid); poly(ferrocenyldimethylsilane) based copolymers such as poly(ferrocenyldimethylsilane-b-ethylene oxide); poly(2-vinyl naphthalene)-based copolymers such as poly(2-vinyl naphthalene-b-acrylic acid), poly (vinyl pyridine and N-methyl vinyl pyridinium iodide)-based copolymers such as poly(2-vinyl pyridine-b-ethylene oxide), poly(2-vinyl pyridine-b-methyl acrylic acid), poly(N-methyl 2-vinyl pyridinium iodide-b-ethylene oxide), poly(N-methyl 4-vinyl pyridinium iodide-b-methyl methacrylate), poly(4-vinyl pyridine-b-ethylene oxide) PEO end functional OH; and poly(vinyl pyrrolidone)-based copolymers such as poly(vinyl pyrrolidone-b-D/L-lactide); and the like.

In an embodiment of the present disclosure, the chromophoric polymer particles provided herein include the polymer CN-PPV, also known as poly[2-methoxy-5-(2-ethylhexyloxy)-1,4-(1-cyanovinylene-1,4-phenylene)], which is a bright, compact, and orange-emitting semiconducting polymer particle. In an embodiment, CN-PPV has superior fluorescence properties, such as a large absorption cross-section, high quantum yield, and a fast emission rate. In an embodiment, the chromophoric polymer particle comprises a polymer that consists essentially of CN-PPV. In an embodiment, the particle includes CN-PPV and at least one other material. For example, the CN-PPV can be mixed with a copolymer or other material that provides an additional functionality.

In an embodiment, the chromophoric polymer particles of the present disclosure include a semiconducting copolymer having at least two different chromophoric units. For example, a conjugated copolymer can contain both fluorene and benzothiazole chromophoric units present at a given ratio. Typical chromophoric units used to synthesize semiconducting copolymers include, but are not limited to fluorene unit, phenylene vinylene unit, phenylene unit, phenylene ethynylene unit, benzothiazole unit, thiophene unit, carbazole fluorene unit, boron-dipyrromethene unit, and derivatives thereof. The different chromophoric units can be segregated, as in a block copolymer, or intermingled. In an embodiment, a chromophoric copolymer is represented by writing the identity of the major chromophoric species. For example, PFBT is a chromophoric polymer containing fluorene and benzothiazole units at a certain ratio. In some cases, a dash is used to indicate the percentage of the minor chromophoric species and then the identity of the minor chromophoric species. For example, PF-0.1 BT is a chromophoric copolymer containing 90% polyfluorene (PF) and 10% benzothiazole (BT).

In an embodiment, the chromophoric polymer particle includes a blend of semiconducting polymers. The blends can include any combination of homopolymers, copolymers, and oligomers. Polymer blends used to form chromophoric polymer particles may be selected in order to tune the properties of the resulting polymer particles, for example, to achieve a desired excitation or emission spectra for the polymer particle.

In various embodiments of the present disclosure, semiconducting chromophoric polymer particles offer improved detection sensitivity in part because they exhibit higher quantum yields than other fluorescent reporters. In an embodiment, the quantum yield of the chromophoric polymer particle used is more than 5%, more than 10%, more than 20%, more than 30%, more than 40%, more than 50%, more than 60%, more than 70%, more than 80%, or more than 90%. In various embodiments, semiconducting chromophoric polymer particles offer improved detection sensitivity in part because they exhibit large absorption cross sections. In various embodiments, semiconducting chromophoric polymer particles offer improved detection sensitivity in part because they exhibit faster emission rates than other fluorescent reporters. In an embodiment, the emission rate of the chromophoric polymer particle used is between about 100 picoseconds and about 50 nanoseconds.

In an embodiment, the chromophoric polymer particles described herein include polymers bearing units of small organic dye molecules, metal complexes, environment-sensing dyes, photochromic dye, and any combinations thereof, for example, optically inactive polymers such as polystyrene covalently linked or grafted with small organic dye, metal complexes, environment-sensing dyes, photochromic dyes, or any combination thereof. In an embodiment, the chromophoric polymer particles comprise semiconducting polymers covalently linked with small organic dye molecules, metal complexes, environment-sensing dyes, photochromic dyes, or any combinations thereof as emissive units. Such emissive units can tune the emission color, increase the quantum yield, or improve the photophysical properties of the chromophoric polymer particle. In an embodiment, the small organic dyes, or metal complexes have sensing functions, and therefore add additional functionalities to the chromophoric polymer particle, such as ion sensing capability.

In an embodiment, the nanoparticle transducer includes one or more chromophores (e.g., fluorophores). The chromophore emits fluorescence that depends on a fluid constituent. In an embodiment, the fluid constituent is a reaction element of a reaction catalyzed by an enzyme of the nanoparticle transducer, the reaction involving an analyte. In some cases, the fluid constituent is a product of the reaction; in some cases, the fluid constituent is a reactant of the reaction. In an embodiment, the reaction rate varies as a function of the analyte concentration, thereby changing the concentration of the fluid constituent and causing the transducer fluorescence to vary accordingly.

In an embodiment, the chromophore comprises a dye. In an embodiment the dye is sensitive to one or more fluid constituents. Examples of dyes that can be used with the nanoparticle transducers disclosed herein include Pt(II)- and Pd(II)-porphyrins, phosphorescent Ru (II) complexes, and Ir (III) complexes. Examples of the dyes include, but not limited to Pt(II) octaethylporphine (PtOEP), Pt(II) meso-tetra(pentafluorophenyl) porphine (PtTFPP), Pt(II) octaethylporphine ketone (PtOEPK), Pd(II) octaethylporphine (PdOEP), and Pd(II) meso-tetra(pentafluorophenyl) porphine (PdTFPP). Pd(II)-meso-tetra-(4-carboxyphenyl)porphyrin (PdTPCPP), Pd(II)-meso-tetra-(4-carboxyphenyl)tetrabenzoporphyrindendrimer (PdTCPTBP), Pt(II)-coproporphyrin (PtCP), Pt(II)-meso-tetrabenzoporphyrin butyl octaester (PtTBP), Pt(II)-coproporphyrin-ketone (PtCPK), cyclometalated Ir(III) 1-chloro-bridged dimer coumarin complex (Ir(III)(Cx)2(acac)), and [Ru(bpy)2(2-(4-carboxyphenyl)imidazo-[4,5-f] [1, 10]phenanthroline)H2)]2+([Ru(bpy)2(picH2)]2+).

In an embodiment, the chromophore comprises a chromophoric unit that is sensitive to ions, pH, reactive oxygen species, reactive nitrogen species, and temperature. In an embodiment, the chromophore comprises a chromophoric unit or dye that is sensitive to ions, pH, reactive oxygen species, reactive nitrogen species, and temperature. Examples of chromophoric unit or dye to construct the nanoparticle transducer include sodium-sensitive, potassium-sensitive, calcium-sensitive, magnesium-sensitive, iron-sensitive, zinc-sensitive, copper-sensitive, manganese-sensitive, pH-sensitive, reactive-oxygen-species-sensitive, reactive-nitrogen-species-sensitive, or temperature-sensitive dye or chromophoric unit. Nanoparticles comprising chromophores sensitive to ions, pH, reactive oxygen species, reactive nitrogen species, and temperature include those described in PCT/US 2010/056079, for example.

In an embodiment, the chromophore comprises a semiconducting chromophoric polymer that is sensitive to one or more fluid constituents. The semiconducting polymer can be designed and synthesized to have fluorescence that is sensitive to one or more fluid constituents.

In an embodiment, the chromophore emits fluorescence that depends on the concentration of NADH and/or NADPH or NAD⁺ and/or NADP⁺, such as is discussed further herein with respect to the EXAMPLES of the present disclosure.

In an embodiment, the chromophore emits fluorescence that depends on the concentration of hydrogen peroxide (H₂O₂). The hydrogen peroxide can be a product reaction element. In an embodiment, the nanoparticle comprises a chromophoric polymer that emits fluorescence that depends on the concentration of hydrogen peroxide. In an embodiment, the nanoparticle comprises a chromophoric polymer and a dye that emits fluorescence at one or more wavelengths. The amount of fluorescence of the dye can depend on the concentration of hydrogen peroxide. The dye can be physically doped or chemically attached to the chromophoric polymer for forming nanoparticles, for example. The chromophoric polymer can have energy transfer between the chromophoric polymer and the dye to enhance or amplify the fluorescence intensity of the dye. Examples of hydrogen peroxide sensitive dyes that can be used with the nanoparticle transducers disclosed herein include Coumarin derivatives, Fluorescein derivatives, Rhodamine derivatives, Cyanine derivatives, Boron-dipyrromethene (BODIPY) derivatives.

In an embodiment, the chromophore emits fluorescence that depends on the concentration of oxygen. The oxygen can be a reactant reaction element. In an embodiment, the nanoparticle comprises a chromophoric polymer that emits fluorescence that depends on the concentration of oxygen. In an embodiment, the nanoparticle comprises a chromophoric polymer and a dye that emits fluorescence at one or more wavelengths. The amount of fluorescence of the dye can depend on the concentration of oxygen. The dye can be physically doped or chemically attached to the chromophoric polymer for forming nanoparticles, for example. The chromophoric polymer can have energy transfer between the chromophoric polymer and the dye to enhance or amplify the fluorescence intensity of the dye. Examples of oxygen sensitive dyes that can be used with the nanoparticle transducers disclosed herein include Pt(II)- and Pd(II)-porphyrins, phosphorescent Ru (II) complexes, and Ir (III) complexes and derivatives thereof.

In an embodiment the chromophore comprises a dye and a semiconducting chromophoric polymer, and the dye and the semiconducting polymer interact to produce enhanced fluorescence. In an embodiment, the semiconducting polymer is not sensitive to the fluid constituents; fluorescence from such a polymer can provide a stable internal standard, thereby acting as a control for signals of variable fluorescence at other wavelengths. The semiconducting chromophoric polymer can have energy transfer to the dye to amplify and enhance the fluorescence of the dye. In an embodiment, the semiconducting polymer is sensitive to the fluid constituents; fluorescence from such a polymer can signal the presence and/or concentration of the analyte. Examples of semiconducting chromophoric polymer that can be used with the nanoparticle transducers disclosed herein include poly(9,9-dihexylfluorene) (PDHF)-based, poly(9,9-dioctylfluorene) (PFO)-based, and poly {[9,9-di-(3-(3-methyloxetan-3-yl)methoxy) hexylfluorenyl-2,7-diyl-co-[9,9-dioctylfluorenyl-2,7-diyl]} (do-PFO)-based, poly[{9,9-dioctyl-2,7-divinylene-fluorenylene}-alt-co-{2-methoxy-5-(2-ethylhexyloxy)-1,4-phenylene}] (PFPV)-based, poly[(9,9-dioctylfluorenyl-2,7-diyl)-co-(1,4-benzo-{2, 1,3}-thiadiazole)] (PFBT)-based, poly[(9,9-dioctylfluorenyl-2,7-diyl)-co-(4,7-Di-2-thienyl-2, 1,3-benzothiadiazole)] (PFTBT)-based, phenylene vinylene polymers, including but not limited to, poly[2-methoxy-5-(2-ethylhexyloxy)-1,4-phenylenevinylene] (MEH-PPV)-based, poly[2-methoxy-5-(2-ethylhexyloxy)-1, 4-(1-cyano vinylene-1,4-phenylene)](CN-PPV)-based, poly(2,5-di(3′,7′-dimethyloctyl)phenylene-1,4-ethynylene (PPE)-based, BODIPY based, and squaraine-based semiconducting polymer and derivatives thereof. In an embodiment the dye is sensitive to one or more fluid constituents.

In an embodiment the chromophore comprises a plurality of dyes. A first dye is sensitive to one or more fluid constituents, and a second dye can interact with the sensitive dye to produce enhanced fluorescence. In an embodiment, at least one dye is not sensitive to the fluid constituents, thus provide a stable fluorescence as an internal standard. The plurality of dyes can emit fluorescence at different wavelengths, allowing independent measurement of each dye's fluorescence. The sensitive and non-sensitive dyes can interact with each other to amplify and enhance the fluorescence of the dye that is sensitive to the one or more fluid constituents.

In an embodiment the chromophore comprises a plurality of semiconducting polymers. In an embodiment, a first semiconducting polymer of the plurality of semiconducting polymers is sensitive to one or more fluid constituents, and a second semiconducting polymer of the plurality of semiconducting polymers is not sensitive to the fluid constituents, thus providing stable fluorescence as an internal standard. The plurality of semiconducting polymers can emit fluorescence at different wavelengths, allowing independent measurement of each semiconducting polymer's fluorescence. In an embodiment the chromophore comprises a semiconducting polymer. In an embodiment, a first monomeric unit of the semiconducting polymer of the plurality of monomeric units of the semiconducting polymer is sensitive to one or more fluid constituents, and a second monomeric unit of the semiconducting polymer of the plurality of monomeric units of the semiconducting polymer is not sensitive to the fluid constituents, thus providing stable fluorescence as an internal standard. The plurality of monomeric units of the semiconducting polymer can emit fluorescence at different wavelengths, allowing measurement of the different semiconducting polymer monomeric unit's fluorescence.

In an embodiment, the chromophoric polymer particle comprises a semiconducting polymer physically mixed or chemically cross-linked with other chromophoric polymers, such as inactive polymers covalently linked or grafted with small organic dye, metal complexes, photochromic dyes, or any combination thereof, to have additional functionalities such as ion sensing or metabolite sensing.

In an embodiment, the chromophoric polymer particle includes semiconducting polymers physically mixed or chemically cross-linked with other components such as fluorescent dyes, inorganic luminescent materials, magnetic materials, metal materials, and the like in order to tune emission color, improve quantum yield and/or photo stability, and/or provide additional functionalities such as magnetic functions, plasmon resonance functions, and the like.

The optical properties, such as absorption wavelength, for a given chromophoric polymer particle can be tuned by modifying its composition and/or structure. Semiconducting polymers have been developed with absorption wavelengths ranging from UV to infrared, including the entire visible spectrum. In an embodiment, chromophoric polymer particles having a peak absorption wavelength between about 200 nanometers and about 300 nanometers, about 250 nanometers and about 350 nanometers, about 300 nanometers and about 400 nanometers, about 350 nanometers and about 450 nanometers, between about 400 nanometers and about 500 nanometers, about 450 nanometers and about 550 nanometers, about 500 nanometers and about 600 nanometers, about 550 nanometers and about 650 nanometers, about 600 nanometers and about 700 nanometers, about 650 nanometers and about 750 nanometers, about 700 nanometers and about 800 nanometers, about 750 nanometers and about 850 nanometers, about 800 nanometers and about 900 nanometers, about 850 nanometers and about 950 nanometers, or about 900 nanometers and about 1000 nanometers, are used.

Semiconducting polymers have been developed with emission wavelengths ranging from UV to infrared, including the entire visible spectrum. In an embodiment, chromophoric polymer particles having a peak emission wavelength between about 200 nanometers and about 300 nanometers, about 250 nanometers and about 350 nanometers, about 300 nanometers and about 400 nanometers, about 350 nanometers and about 450 nanometers, about 400 nanometers and about 500 nanometers, about 450 nanometers and about 550 nanometers, about 500 nanometers and about 600 nanometers, about 550 nanometers and about 650 nanometers, about 600 nanometers and about 700 nanometers, about 650 nanometers and about 750 nanometers, about 700 nanometers and about 800 nanometers, about 750 nanometers and about 850 nanometers, about 800 nanometers and about 900 nanometers, about 850 nanometers and about 950 nanometers, about 900 nanometers and about 1000 nanometers, about 950 nanometers and about 1050 nanometers, about 1000 nanometers and about 1100 nanometers, about 1150 nanometers and about 1250 nanometers, or about 1200 nanometers and about 1300 nanometers, are used.

In an embodiment, the present disclosure provides one or more chromophores with narrow-band emissions. Narrow-band emissions are advantageous for certain applications, including but not limited to resolution of multiple fluorescence signals. The emission wavelength of the one or more chromophores can vary from ultraviolet to near infrared region. In an embodiment, the FWHM of the emission band is less than about 100 nanometers, about 70 nanometers, about 65 nanometers, about 60 nanometers, about 55 nanometers, about 50 nanometers, about 45 nanometers, about 40 nanometers, about 35 nanometers, about 30 nanometers, about 25 nanometers, about 20 nanometers, or about 10 nanometers. In an embodiment, the FWHM of the polymer particles described herein can range between about 5 nanometers to about 100 nanometers, from about 10 nanometers to about 70 nanometers, from about 20 nanometers to about 60 nanometers, or from about 30 nanometers to about 50 nanometers.

In an embodiment, the variety of one or more chromophores of the present disclosure include polymers that have a narrow band emissive unit (e.g., a narrow band repeating unit and/or a narrow band unit). For example, the present disclosure can include a homopolymer or heteropolymer including a narrow band repeating unit, such as BODIPY and/or BODIPY derivative repeating unit, a squaraine and/or squaraine derivative repeating unit, a metal complex and/or metal complex derivative repeating unit, a porphyrin and/or porphyrin derivative repeating unit, a metalloporphyrin and/or metalloporphyrin derivative repeating unit, a phthalocyanine and/or phthalocynanine derivative repeating unit, a lanthanide complex and/or lanthanide complex derivative repeating unit, a perylene and/or perylene derivative repeating unit, a cyanine and/or cyanine derivative repeating unit, a rhodamine and/or rhodamine derivative repeating unit, a coumarin and/or coumarin derivative repeating unit, and/or a xanthene and/or xanthene derivative repeating unit. In an embodiment, a narrow band unit is, e.g., a narrow band repeating unit or a fluorescent nanoparticle embedded in or attached to the polymer particle. The one or more chromophores can include, e.g., a quantum dot. Optionally, a narrow band unit includes a polymer or fluorescent dye molecule that gives a narrow emission in a polymer particle of the present disclosure.

In some embodiments, the chemical composition and structure of the one or more chromophores can affect the absorption spectrum of the one or more chromophores. The absorption peak can shift from ultra-violet region to infrared region. In some embodiments, the absorption peak of the one or more chromophores can be tuned to a certain laser wavelength. In some embodiments, for example, the absorption peak can be tuned to 405 nm. In some embodiments, the absorption peak can be tuned to around 450 nm. In some embodiments, the absorption peak can be tuned to around 488 nm. In some embodiments, the absorption peak can be tuned to around 532 nm. In some embodiments, the absorption peak can be tuned to around 561 nm. In some embodiments, the absorption peak can be tuned to around 633 nm. In some embodiments, the absorption peak can be tuned to around 635 nm. In some embodiments, the absorption peak can be tuned to around 640 nm. In some embodiments, the absorption peak can be tuned to around 655 nm. In some embodiments, the absorption peak can be tuned to around 700 nm. In some embodiments, the absorption peak can be tuned to around 750 nm. In some embodiments, the absorption peak can be tuned to around 800 nm. In some embodiments, the absorption peak can be tuned to around 850 nm. In some embodiments, the absorption peak can be tuned to around 900 nm. In some embodiments, the absorption peak can be tuned to around 980 nm. In some embodiments, the absorption peak can be tuned to the near-infrared region of the wavelength spectrum (e.g., from 750 nm to 1200 nm). In some embodiments, the absorption peak can be tuned to around 1064 nm. In some embodiments, for example, the absorption peak can be tuned to between 380 and 420 nm. In some embodiments, the absorption peak can be tuned to between 440 and 460 nm. In some embodiments, the absorption peak can be tuned to between 478 and 498 nm. In some embodiments, the absorption peak can be tuned to between 522 and 542 nm. In some embodiments, the absorption peak can be tuned to between 550 and 570 nm. In some embodiments, the absorption peak can be tuned to between 625 and 645 nm. In some embodiments, the absorption peak can be tuned to between 645 and 665 nm. In some embodiments, the absorption peak can be tuned to between 690 and 710 nm. In some embodiments, the absorption peak can be tuned to between 740 and 760 nm. In some embodiments, the absorption peak can be tuned to between 790 and 810 nm. In some embodiments, the absorption peak can be tuned to between 890 and 910 nm. In some embodiments, the absorption peak can be tuned to between 970 and 990 nm. In some embodiments, the absorption peak can be tuned to between 1054 and 1074 nm.

In certain embodiments the chromophore absorbance width is measured at from 20% to 16% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 20% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 19% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 18% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 17% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 16% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 15% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 14% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 13% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 12% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 11% of the absorbance maximum. In some embodiments the chromophore has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 10% of the absorbance maximum.

In an embodiment of the present disclosure, the apparatus, compositions, systems, and methods provided herein utilize one or more chromophores (e.g., dyes or semiconducting chromophoric polymers) that are capable of generating fluorescent light at one or more wavelengths, for example, in response to incident radiation such as UV, visible, far red, near infrared, or other light. In some cases, amount of fluorescence from the chromophore at a given wavelength varies as a function of the local concentration of a fluid constituent (a signal chromophore); in other aspects, amount of fluorescence from the chromophore does not vary in response to said local concentration (a control chromophore). In an embodiment, nanoparticles as provided herein can incorporate both a signal and a control chromophore, emitting fluorescence at a signal wavelength and a control wavelength, respectively. Although various embodiments herein are described in the context of nanoparticles having one or two different emission wavelengths, it shall be understood that the approaches presented herein are also applicable to nanoparticles that emit more than two wavelengths. For example, nanoparticles can be provided that emit at two signal wavelengths and one or two control wavelengths, which can be used for multiple analyte measurement signals. A plurality of different nanoparticles with different signal/control wavelength pairs, each responsive to a distinct analyte (or optionally to the same analyte, e.g., for redundant signaling), may be provided.

In an embodiment, the chromophores generating fluorescence at the signal wavelength exhibit different optical characteristics (e.g., emission spectrum, absorbance spectrum, peak emission wavelength(s), peak excitation wavelength(s), emission intensities, emission lifetimes, emission rates) when in different concentrations of a fluid constituent. For instance, chromophore may exhibit increased (or decreased) fluorescence in response to increased concentration of a fluid constituent such as a molecule. In an embodiment, the variation of the fluorescence can be ratiometric as a function of the concentration of a fluid constituent. The molecule can be oxygen or NADH or NADPH, or NAD⁺ or NADP⁺ or hydrogen peroxide, for example, which can be a reactant or a product of a reaction involving an analyte to be measured and catalyzed by an enzyme. The enzyme can be physically associated or coupled to a nanoparticle comprising the chromophore, such that reactions catalyzed by the enzyme change the local concentration of the molecule, thereby changing the fluorescence of the chromophore in response to changes in concentration of the analyte. In an embodiment, such changes can be ratiometric. Fluorescence can be generated at a control wavelength, such that a fluorescence ratio of control to signal can serve as a signal of analyte concentration, eliminating or reducing certain sources of noise and uncertainty in fluorescence intensity measurements, for example.

Enzyme Compositions

In an embodiment disclosed herein, small molecule detection is provided based on the integration of an enzyme, such as an NADH-dependent or NADPH-dependent enzyme, that catalyzes the small molecule reactions and which comprise a plurality of reaction elements, and a chromophore configured to emit fluorescence based on a concentration of a reaction element, such as NADH or NADPH, of the plurality of reaction elements. In an embodiment disclosed herein, detection of a molecule (e.g. lipid, carbohydrate, protein, nucleic acid, metabolite, peptide, drug, substrate of an enzyme) is provided based on the integration of an enzyme, such as an NADH-dependent or NADPH-dependent enzyme, that catalyzes the molecule reactions and which comprise a plurality of reaction elements, and a chromophore configured to emit fluorescence based on a concentration of a reaction element, such as NADH or NADPH, of the plurality of reaction elements. In an embodiment disclosed herein, small molecule detection is provided based on the integration of a transducer with an enzyme, such as an NADH-dependent or NADPH-dependent enzyme, that catalyzes the small molecule reactions.

In some cases, the chromophore can be directly mixed with the enzyme for measurements. In some cases, covalent conjugation is provided to link the nanoparticle to the enzyme, yielding a compact probe that can be used for, for example, intracellular sensing. In an embodiment, the enzyme is physically associated with the chromophore. As discussed further herein, such physical association can include an enzyme dispersed in a common solvent with a chromophore, an enzyme coupled to a common substrate with a chromophore, an enzyme lyophilized in a common powder with a chromophore, an enzyme encapsulated with a chromophore in a hydrogel bead, or an enzyme otherwise in physical/chemical contact with a chromophore. In an embodiment, the enzyme physically associated with the chromophore is coupled to the chromophore. In this regard, the enzyme is directly or indirectly physically connected to the chromophore. Such coupling can include a covalent bond. In another embodiment, the coupling between an enzyme and a chromophore is through one or more non-covalent bonds or interactions, such as an ionic bond, van der Waals forces, hydrogen bonding, and the like. Accordingly, in an embodiment, the coupling does not include a covalent bond.

As discussed elsewhere herein, the chromophores can take a number of different forms and comprise a number of different types of chromophores. In an embodiment, the chromophore is the form of a nanoparticle. In an embodiment, the chromophore is a semiconducting polymer, such as in the form of a semiconducting polymer nanoparticle. As described elsewhere herein, in an embodiment, the chromophore is part of a polymer dot or pdot, such as where the chromophore and other polymeric components are present in a condensed, stable, submicron state. In an embodiment, the chromophore can include a chromophoric polymer, such as a semiconducting chromophoric polymer, in an uncondensed state.

Upon formation of the enzyme corona on a nanoparticle surface, the nanoparticle-enzyme bioconjugate behaves as a nanoreactor that depletes or produces, for example, NAD⁺ or NADP⁺, or NADH or NADPH in the presence of the small molecule analyte to which the enzyme is sensitive. Small molecule concentrations are thus monitored by the optical signal of the transducer as an analyte is depleted or produced. The performance of this sensing scheme is dependent on factors including: (1) whether the presence of the analyte can induce distinct changes in reaction-element distribution profile; (2) whether the transducer is able to transform the analyte concentration change to optical signal. In addition, the in vivo detection is also closely related with the issues such as local microvascular perfusion, availability of analyte in tissue, and enzyme activity. In the following EXAMPLES, NAD⁺/NADH is provided as an example by which the effectiveness of the transducers described herein in sensing analyte concentrations may be illustrated with both theoretical analysis and experimental evidence, for both in vitro and in vivo applications. Based on the examples described herein, transducers may be fabricated to produce fluorescent signals for detection of a wide variety of analytes, including small molecules, large molecules, and other fluid constituents, through the selection of an appropriately-reactive enzyme and corresponding chromophores sensitive to a reaction element of the reaction catalyzed thereby.

In an embodiment, the nanoparticle transducers provided herein comprise an enzyme, and the enzyme catalyzes a reaction. The reaction involves the analyte to be measured, and produces products and consumes reactants, collectively referred to as reaction elements. In an embodiment, the reaction elements include a fluid constituent, and a concentration of the fluid constituent is changed by the reaction. For example, the fluid constituent can be a reaction product and the reaction can increase the concentration of the fluid constituent. Alternatively, the fluid constituent can be a reactant and reaction can decrease its concentration.

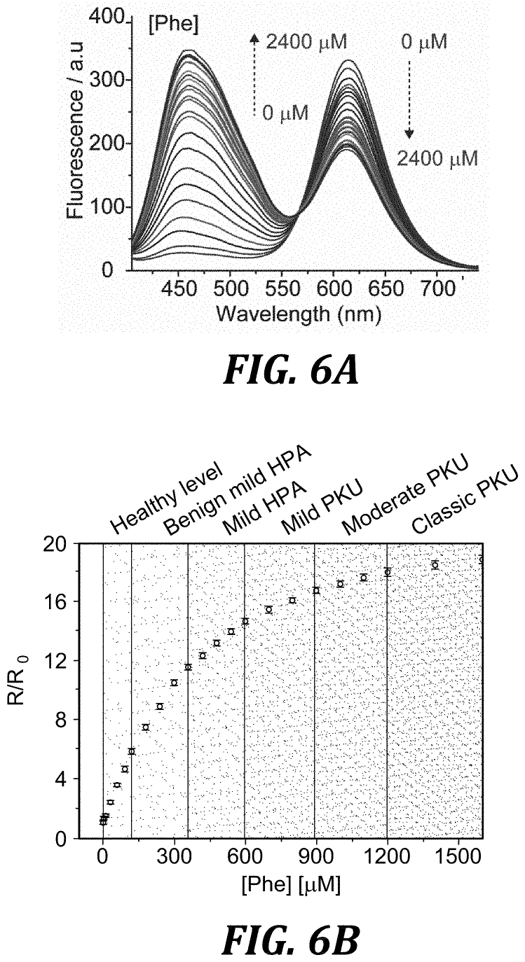

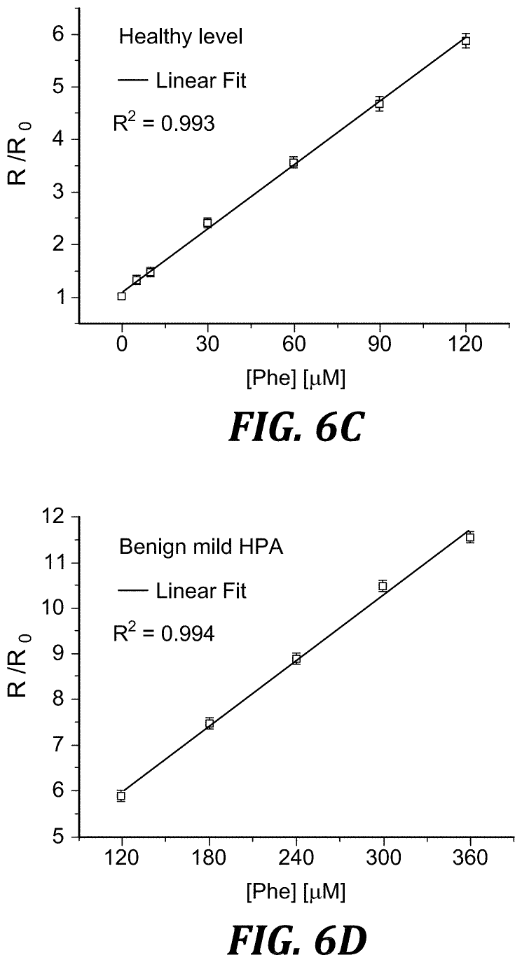

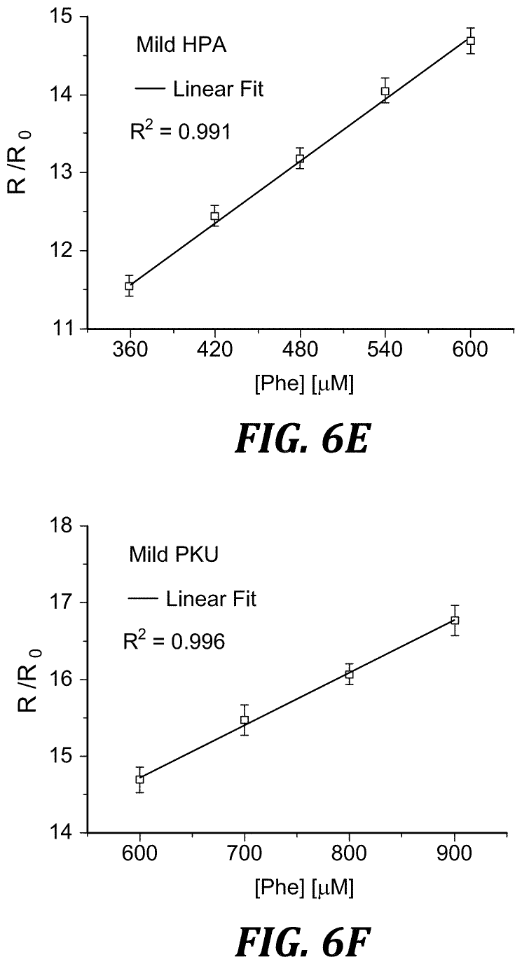

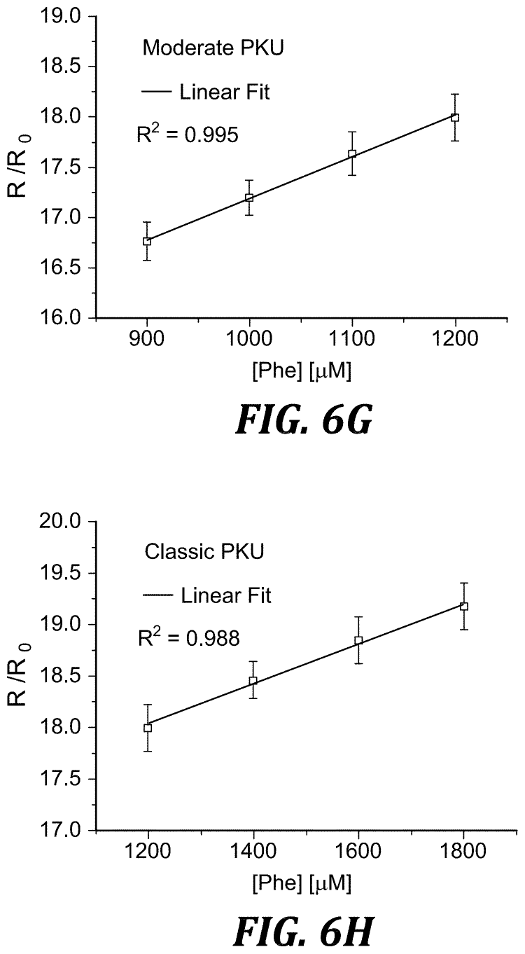

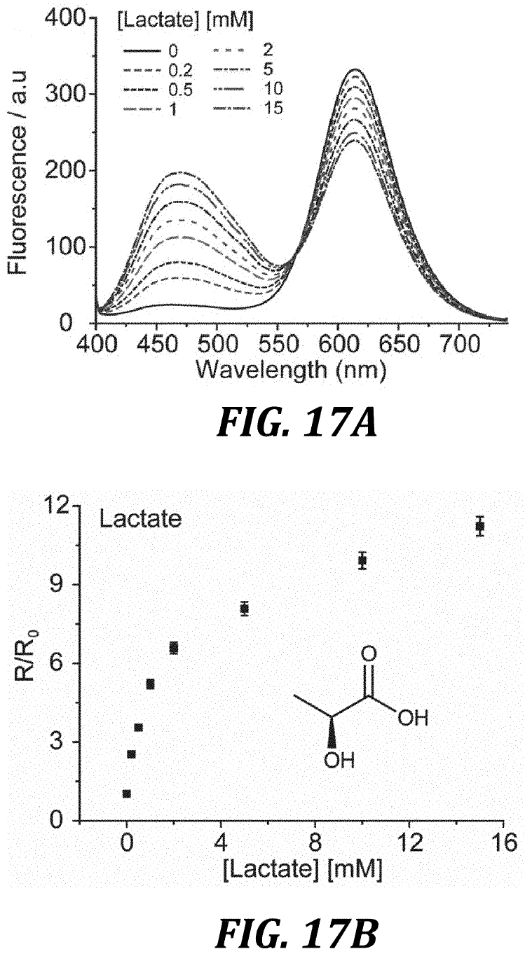

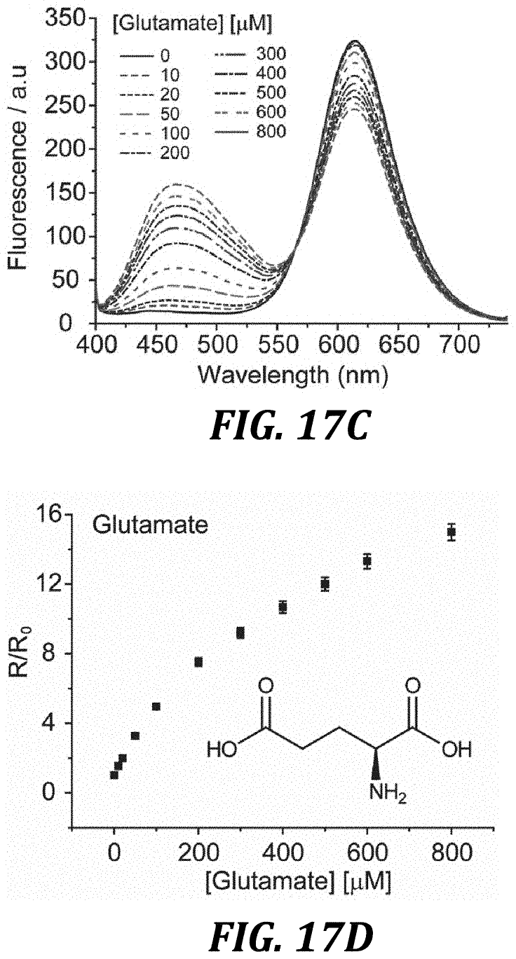

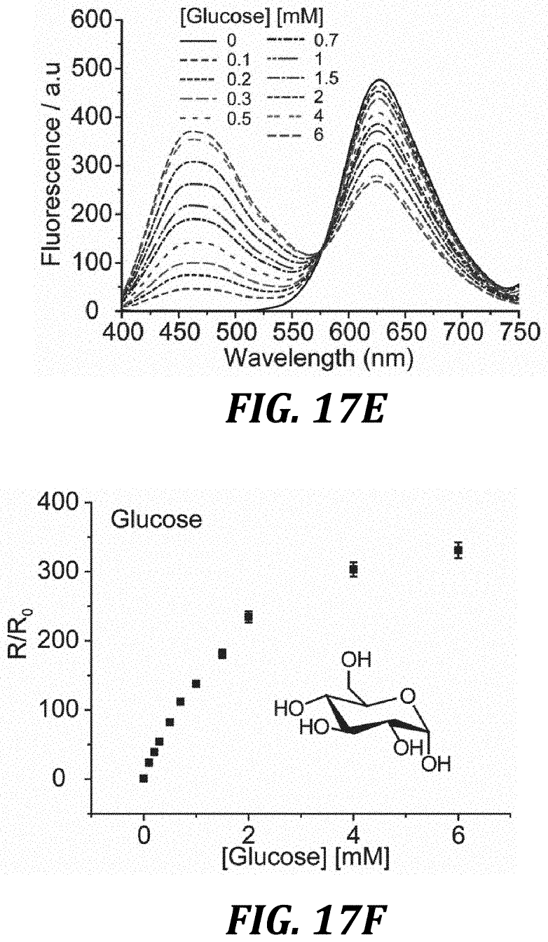

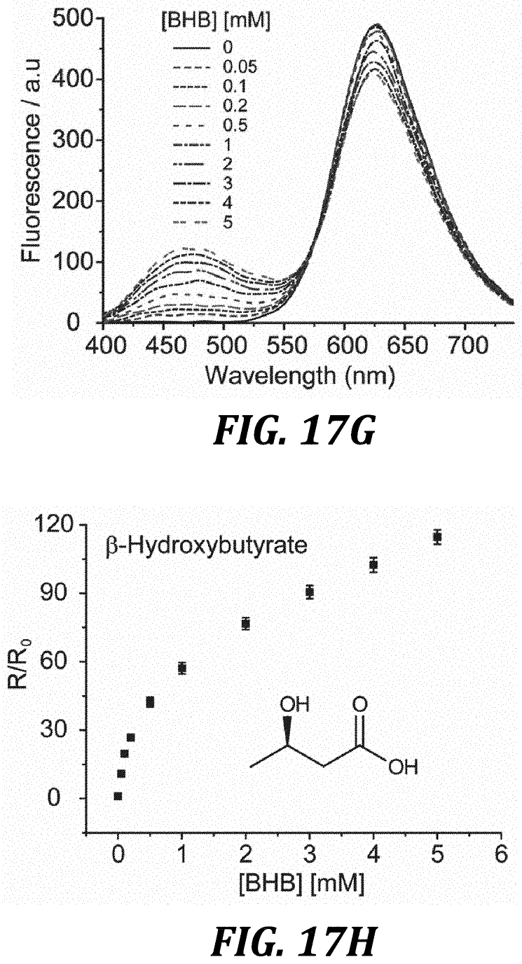

In an embodiment, the fluid constituent is NADH, and the NADH is a product. In an embodiment, the fluid constituent is NADH, and the NADH is a reactant. In an embodiment, the fluid constituent is NAD⁺, and the NAD⁺ is a reactant. In an embodiment, the fluid constituent is NAD⁺, and the NAD⁺ is a product. In an embodiment, the fluid constituent is NADPH, and the NADPH is a product. In an embodiment, the fluid constituent is NADPH, and the NADPH is a reactant. In an embodiment, the fluid constituent is NADP⁺, and the NADP⁺ is a reactant. In an embodiment, the fluid constituent is NADP⁺, and the NADP⁺ is a product. In an embodiment, the NADH-dependent or NADPH-dependent enzyme and analyte, respectively, comprise one or more of the following pairs: phenylalanine and phenylalanine dehydrogenase (see FIGS. 6A and 6B), lactate and lactate dehydrogenase (see FIGS. 17A and 17B); glutamate and glutamate dehydrogenase (see FIGS. 17C and 17D); glucose and glucose dehydrogenase (see FIGS. 17E and 17F); and β-hydroxybutyrate (BHB) and BHB dehydrogenase (see FIGS. 17G and 17H).

In an embodiment, the fluid constituent is other than NADH; for example, the fluid constituent can be an ion, the enzyme can catalyze a reaction that changes ionic concentration, and the chromophore can produce fluorescence modulated by said ionic concentration; the fluid constituent can be an acid or base, the enzyme can catalyze a reaction that changes pH, and the chromophore can produce fluorescence modulated by said pH; or the fluid constituent can be heat energy, the enzyme can catalyze a reaction that changes temperature, and the chromophore can produce fluorescence modulated by said temperature. In an embodiment, the fluid constituent can be hydrogen peroxide, the enzyme can catalyze a reaction that changes hydrogen peroxide concentration, and the chromophore can produce fluorescence modulated by said hydrogen peroxide concentration. For example, hydrogen peroxide can be a product of the reaction. In an embodiment, the fluid constituent is oxygen, the enzyme can catalyze a reaction that changes oxygen concentration, and the chromophore can produce fluorescence modulated by said oxygen concentration. For example, oxygen can be a reactant of the reaction.

In an embodiment, a plurality of enzymes is coupled to the nanoparticle transducer to catalyze a respective plurality of reactions. The plurality of reactions forms a reaction chain, where one or more products of one reaction are reactants of another reaction. For example, an enzyme cascade may be provided by the plurality of enzymes, with each enzyme performing a step of the cascade. At least one of the plurality of reactions involves the analyte as a reactant, and at least one of the reactions has the fluid constituent as a reaction element for modulation of the chromophore emission intensity.

In an embodiment, the enzyme is an NADH-dependent or NADPH-dependent enzyme. In an embodiment, the NADH-dependent or NADPH-dependent enzyme is selected from the group consisting a dehydrogenase, a reductase, an oxygenase, a synthase, a hydroxylase, and combinations thereof. In an embodiment, the NADH-dependent or NADPH-dependent enzyme is a dehydrogenase. In an embodiment, the dehydrogenase is selected from the group consisting of (−)-menthol dehydrogenase; (+)-neomenthol dehydrogenase; (+)-sabinol dehydrogenase; (+)-trans-carveol dehydrogenase; (3S,4R)-3,4-dihydroxycyclohexa-1,5-diene-1,4-dicarboxylate dehydrogenase; (R)-2-hydroxy-fatty-acid dehydrogenase; (R)-2-hydroxyacid dehydrogenase; (R)-4-hydroxyphenyllactate dehydrogenase; (R)-aminopropanol dehydrogenase; (R)-dehydropantoate dehydrogenase; (S)-2-hydroxy-fatty-acid dehydrogenase; (S)-carnitine 3-dehydrogenase; 1,2-dihydroxy-6-methylcyclohexa-3,5-dienecarboxylate dehydrogenase; 1,3-propanediol dehydrogenase; 1,6-dihydroxycyclohexa-2,4-diene-1-carboxylate dehydrogenase; 2-alkyn-1-ol dehydrogenase; 2-dehydro-3-deoxy-D-gluconate 5-dehydrogenase; 2-deoxy-D-gluconate 3-dehydrogenase; 2-hydroxymethylglutarate dehydrogenase; 2-hydroxypropyl-CoM dehydrogenase; 2-oxoaldehyde dehydrogenase; 2-oxoisovalerate dehydrogenase; 2,3-dihydro-2,3-dihydroxybenzoate dehydrogenase; 2,3-dihydroxy-2,3-dihydro-p-cumate dehydrogenase; 2,4-diaminopentanoate dehydrogenase; 2,5-dioxovalerate dehydrogenase; 3-(imidazol-5-yl)lactate dehydrogenase; 3-α-hydroxy-5β-androstane-17-one 3α-dehydrogenase; 3-α-hydroxycholanate dehydrogenase; 3-α-hydroxyglycyrrhetinate dehydrogenase; 3-α-hydroxysteroid dehydrogenase; 3-α(17beta)-hydroxysteroid dehydrogenase; 3-α(or 20beta)-hydroxysteroid dehydrogenase; 3-beta-hydroxy-5α-steroid dehydrogenase; 3-beta-hydroxy-5beta-steroid dehydrogenase; 3-beta-hydroxysteroid dehydrogenase; 3-beta(or 20α)-hydroxysteroid dehydrogenase; 3-dehydro-L-gulonate 2-dehydrogenase; 3-hydroxy-2-methylbutyryl-CoA dehydrogenase; 3-hydroxyacid-ester dehydrogenase; 3-hydroxyacyl-CoA dehydrogenase; 3-hydroxybenzyl-alcohol dehydrogenase; 3-hydroxyisobutyrate dehydrogenase; 3-hydroxypimeloyl-CoA dehydrogenase; 3-hydroxypropionate dehydrogenase; 3-Isopropylmalate dehydrogenase; 3(or 17)a-hydroxysteroid dehydrogenase; 4-(hydroxymethyl)benzenesulfonate dehydrogenase; 4-carboxy-2-hydroxymuconate-6-semialdehyde dehydrogenase; 4-formylbenzenesulfonate dehydrogenase; 4-hydroxybenzaldehyde dehydrogenase; 4-hydroxybutyrate dehydrogenase; 4-hydroxycyclohexanecarboxylate dehydrogenase; 4-hydroxymuconic-semialdehyde dehydrogenase; 4-hydroxyphenylacetaldehyde dehydrogenase; 4-hydroxythreonine-4-phosphate dehydrogenase; 4-phosphoerythronate dehydrogenase; 4-trimethylammoniobutyraldehyde dehydrogenase; 5-carboxymethyl-2-hydroxymuconic-semialdehyde dehydrogenase; 5,6-dihydroxy-3-methyl-2-oxo-1,2,5,6-tetrahydroquinoline dehydrogenase; 5-hydroxyeicosanoid dehydrogenase; 6-endo-hydroxycineole dehydrogenase; 6-hydroxyhexanoate dehydrogenase; 6-oxocineole dehydrogenase; 6-oxohexanoate dehydrogenase; 6-phosphogluconic dehydrogenase; 7-α-hydroxysteroid dehydrogenase; 7-β-hydroxysteroid dehydrogenase; 11-β-hydroxysteroid dehydrogenase; 12-α-hydroxysteroid dehydrogenase; 12-β-hydroxysteroid dehydrogenase; 15-hydroxyicosatetraenoate dehydrogenase; 15-hydroxyprostaglandin dehydrogenase; 16-α-hydroxysteroid dehydrogenase; 17-β-hydroxysteroid dehydrogenase; 20-α-hydroxysteroid dehydrogenase; 21-hydroxysteroid dehydrogenase; Acyl-CoA dehydrogenase; Alanine dehydrogenase; Alanopine dehydrogenase; α-ketoglutarate dehydrogenase; α-glycerophosphate dehydrogenase; Alcohol dehydrogenase; Aldehyde dehydrogenase; Aldose 1-dehydrogenase; Allyl-alcohol dehydrogenase; Aminobutyraldehyde dehydrogenase; Aminomuconate-semialdehyde dehydrogenase; Arogenate dehydrogenase; Aryl-alcohol dehydrogenase; Aryl-aldehyde dehydrogenase; Aspartate dehydrogenase; Aspartate-semialdehyde dehydrogenase; Benzaldehyde dehydrogenase; Benzyl-2-methyl-hydroxybutyrate dehydrogenase; β-alanopine dehydrogenase; β-hydroxybutyrate dehydrogenase; betaine-aldehyde dehydrogenase; Bomeol dehydrogenase; Butanal dehydrogenase; Butanediol dehydrogenase; Carnitine 3-dehydrogenase; Carveol dehydrogenase; Cholest-5-ene-30,7α-diol 30-dehydrogenase; Cholestanetetraol 26-dehydrogenase; Cholesterol dehydrogenase; Cinnamyl-alcohol dehydrogenase; Cis-1,2-dihydro-1,2-dihydroxynaphthalene dehydrogenase; Cis-1,2-dihydrobenzene-1,2-diol dehydrogenase; Cis-1,2-dihydroxy-4-methylcyclohexa-3,5-diene-1-carboxylate dehydrogenase; Cis-2,3-dihydrobiphenyl-2,3-diol dehydrogenase; Cis-3,4-dihydrophenanthrene-3,4-diol dehydrogenase; Cis-dihydroethylcatechol dehydrogenase; Coniferyl-alcohol dehydrogenase; Coniferyl-aldehyde dehydrogenase; Cyclohexane-1,2-diol dehydrogenase; Cyclohexanol dehydrogenase; Cyclopentanol dehydrogenase; D-arabinitol 2-dehydrogenase; D-arabinitol 4-dehydrogenase; D-arabinose 1-dehydrogenase; D-lysopine dehydrogenase; D-iditol 2-dehydrogenase; D-malate dehydrogenase; D-nopaline dehydrogenase; D-pinitol dehydrogenase; D-threo-aldose 1-dehydrogenase; D-xylose 1-dehydrogenase; Diaminopimelate dehydrogenase; Dibenzothiophene dihydrodiol dehydrogenase; Dihydrobunolol dehydrogenase; Dihydropyrimidine dehydrogenase; Dihydrouracil dehydrogenase; Dimethylmalate dehydrogenase; DTDP-6-deoxy-L-talose 4-dehydrogenase; DTDP-galactose 6-dehydrogenase; Ephedrine dehydrogenase; Erythrose-4-phosphate dehydrogenase; Estradiol 17-α-dehydrogenase; Estradiol 17-β-dehydrogenase; Farnesol dehydrogenase; Fluoren-9-ol dehydrogenase; Fluoroacetaldehyde dehydrogenase; Formaldehyde dehydrogenase; Formate dehydrogenase; Formyltetrahydrofolate dehydrogenase; Fructose dehydrogenase; Galactitol 2-dehydrogenase; Galactitol-1-phosphate 5-dehydrogenase; Galactose dehydrogenase; Gamma-guanidinobutyraldehyde dehydrogenase; GDP-6-deoxy-D-talose 4-dehydrogenase; GDP-mannose 6-dehydrogenase; Geissoschizine dehydrogenase; Geraniol dehydrogenase; Gluconate 2-dehydrogenase; Gluconate 5-dehydrogenase; Glucose dehydrogenase; Glucose-6-phosphate dehydrogenase; Glutamic (glutamate) dehydrogenase; Glutamate-5-semialdehyde dehydrogenase; Glutarate-semialdehyde dehydrogenase; Glyceraldehyde-3-phosphate dehydrogenase; Glycerate dehydrogenase; Glycerol dehydrogenase; Glycerol 2-dehydrogenase; Glycerol-3-phosphate dehydrogenase; Glycine dehydrogenase; Glycolaldehyde dehydrogenase; Glyoxylate dehydrogenase; Hexadecanol dehydrogenase; Histidinol dehydrogenase; Homoisocitrate dehydrogenase; Homoserine dehydrogenase; Hydrogen dehydrogenase; Hydroxycyclohexanecarboxylate dehydrogenase; Hydroxymalonate dehydrogenase; Hypotaurine dehydrogenase; Indanol dehydrogenase; Indolelactate dehydrogenase; Inosine-5′-monophosphate dehydrogenase; Inositol 2-dehydrogenase; Isocitric (isocitrate) dehydrogenase; Isopiperitenol dehydrogenase; Isopropanol dehydrogenase; Kynurenate-7,8-dihydrodiol dehydrogenase; L-amino-acid dehydrogenase; L-aminoadipate-semialdehyde dehydrogenase; L-arabinitol 2-dehydrogenase; L-arabinitol 4-dehydrogenase; L-arabinose 1-dehydrogenase; L-arginine dehydrogenase; L-erythro-3,5-diaminohexanoate dehydrogenase; L-glycol dehydrogenase; L-gulonate 3-dehydrogenase; L-iditol 2-dehydrogenase; L-idonate 5-dehydrogenase; L-rhamnose 1-dehydrogenase; L-threonate 3-dehydrogenase; L-threonine 3-dehydrogenase; L-xylose 1-dehydrogenase; Lactaldehyde dehydrogenase; Lactic (lactate) dehydrogenase; Leucine dehydrogenase; Long-chain-alcohol dehydrogenase; Lysine dehydrogenase; Malic (malate) dehydrogenase; Malonate-semialdehyde dehydrogenase; Mannitol 2-dehydrogenase; Mannitol dehydrogenase; Mannitol-1-phosphate 5-dehydrogenase; Meso-tartrate dehydrogenase; Methylenetetrahydrofolate dehydrogenase; Methylmalonate-semialdehyde dehydrogenase; Morphine 6-dehydrogenase; Mycothiol-dependent formaldehyde dehydrogenase; N-acetylhexosamine 1-dehydrogenase; N-acylmannosamine 1-dehydrogenase; N-methylalanine dehydrogenase; NADH dehydrogenase; NADPH dehydrogenase; Nicotinate dehydrogenase; Octanol dehydrogenase; Omega-hydroxydecanoate dehydrogenase; Opine dehydrogenase; Oxoglutarate dehydrogenase; Pantoate 4-dehydrogenase; Perillyl-alcohol dehydrogenase; Phenylacetaldehyde dehydrogenase; Phenylalanine dehydrogenase; Phenylglyoxylate dehydrogenase; Phosphogluconate dehydrogenase; Phosphoglycerate dehydrogenase; Phosphonate dehydrogenase; Phthalate 4,5-cis-dihydrodiol dehydrogenase; Pimeloyl-CoA dehydrogenase; Precorrin-2 dehydrogenase; Prephenate dehydrogenase; Propanediol-phosphate dehydrogenase; Pyridoxal 4-dehydrogenase; Pyridoxine 4-dehydrogenase; Pyruvate dehydrogenase; Quinate dehydrogenase; Retinal dehydrogenase; Retinol dehydrogenase; Ribitol 2-dehydrogenase; Ribitol-5-phosphate 2-dehydrogenase; Ribose 1-dehydrogenase; S-(hydroxymethyl)glutathione dehydrogenase; Saccharopine dehydrogenase; Salicylaldehyde dehydrogenase; Sequoyitol dehydrogenase; Serine 2-dehydrogenase; Serine 3-dehydrogenase; Shikimate dehydrogenase; Sn-glycerol-1-phosphate dehydrogenase; Sorbitol-6-phosphate 2-dehydrogenase; Sorbose 5-dehydrogenase; Sterol-4α-carboxylate 3-dehydrogenase; Strombine dehydrogenase; Succinate-semialdehyde dehydrogenase; Succinylglutamate-semialdehyde dehydrogenase; Tartrate dehydrogenase; Tauropine dehydrogenase; Terephthalate 1,2-cis-dihydrodiol dehydrogenase; Testosterone 170-dehydrogenase; Thiomorpholine-carboxylate dehydrogenase; Trans-1,2-dihydrobenzene-1,2-diol dehydrogenase; Trans-acenaphthene-1,2-diol dehydrogenase; Tryptophan dehydrogenase; UDP-glucose 6-dehydrogenase; UDP-N-acetylglucosamine 6-dehydrogenase; UDP-N-acetylmuramate dehydrogenase; Ureidoglycolate dehydrogenase; Uronate dehydrogenase; Valine dehydrogenase; Vanillin dehydrogenase; Vellosimine dehydrogenase; Vomifoliol dehydrogenase; Xanthine dehydrogenase; and Xanthoxin dehydrogenase.

In an embodiment, the NADH-dependent or NADPH-dependent enzyme is a reductase. In an embodiment, the reductase is selected from the group consisting of (S)-usnate reductase; 1,2-dehydroreticulinium reductase; 1,2-dihydrovomilenine reductase; 1,5-anhydro-D-fructose reductase; 2-alkenal reductase; 2-coumarate reductase; 2-dehydropantoate 2-reductase; 2-dehydropantolactone reductase; 2-enoate reductase; 2-hexadecenal reductase; 2-hydroxy-1,4-benzoquinone reductase; 2-hydroxy-3-oxopropionate reductase; 2-hydroxy-6-oxo-6-phenylhexa-2,4-dienoate reductase; 2-oxoadipate reductase; 2-oxopropyl-CoM reductase; 2,4-dichlorobenzoyl-CoA reductase; 2,5-didehydrogluconate reductase; 2′-hydroxydaidzein reductase; 2′-hydroxyisoflavone reductase; 3″-deamino-3″-oxonicotianamine reductase; 3-dehydrosphinganine reductase; 3-ketosteroid reductase; 3-methylbutanal reductase; 3-methyleneoxindole reductase; 3-oxoacyl-(acyl-carrier-protein) reductase; 4-(dimethylamino)phenylazoxybenzene reductase; 4-hydroxy-tetrahydrodipicolinate reductase; 4-oxoproline reductase; 5-amino-6-(5-phosphoribosylamino)uracil reductase; 6-pyruvoyltetrahydropterin 2′-reductase; 6,7-dihydropteridine reductase; 8-oxocoformycin reductase; 12-oxophytodienoate reductase; Acetoacetyl-CoA reductase; Acylglycerone-phosphate reductase; Aldose reductase; Aldose-6-phosphate reductase; α-santonin 1,2-reductase; Anthocyanidin reductase; Apiose 1-reductase; Aquacobalamin reductase; Asparagusate reductase; Azobenzene reductase; Berberine reductase; β-nitroacrylate reductase; Biliverdin reductase; Biochanin-A reductase; Bis-gamma-glutamylcystine reductase; Carbonyl reductase; CDP-4-dehydro-6-deoxyglucose reductase; Chlordecone reductase; Cholestenone 5α-reductase; Cinnamoyl-CoA reductase; Cis-2-enoyl-CoA reductase; CoA-glutathione reductase; CoA-disulfide reductase; Cob(II)alamin reductase; Codeinone reductase; Cortisone α-reductase; Cucurbitacin Delta23-reductase; Cyanocobalamin reductase; Cystine reductase; D-xylulose reductase; Delta1-piperideine-2-carboxylate reductase; Delta14-sterol reductase; Delta24-sterol reductase; Delta24(241)-sterol reductase; Diethyl 2-methyl-3-oxosuccinate reductase; Diferric-transferrin reductase; Dihydrokaempferol 4-reductase; Diiodophenylpyruvate reductase; Divinyl chlorophyllide a 8-vinyl-reductase; DTDP-4-dehydro-6-deoxyglucose reductase; DTDP-4-dehydrorhamnose reductase; Enoyl-(acyl-carrier-protein) reductase; Erythrulose reductase; Ferredoxin—NAD⁺ reductase; Ferric-chelate reductase; Flavanone 4-reductase; Flavin reductase; FMN reductase; Fructuronate reductase; Fumarate reductase; GDP-4-dehydro-6-deoxy-D-mannose reductase; GDP-4-dehydro-D-rhamnose reductase; Glucuronate reductase; Glucuronolactone reductase; Glutamyl-tRNA reductase; Glyoxylate reductase; Hydroxylamine reductase; Hydroxymethylglutaryl-CoA reductase; Hydroxyphenylpyruvate reductase; Hydroxypyruvate reductase; Hyponitrite reductase; Indole-3-acetaldehyde reductase; L-xylulose reductase; Lactaldehyde reductase; Leghemoglobin reductase; Leucoanthocyanidin reductase; Long-chain-fatty-acyl-CoA reductase; Maleylacetate reductase; Mannose-6-phosphate 6-reductase; Mannuronate reductase; Mercury(II) reductase; (Methionine synthase) reductase; Methylglyoxal reductase; Mevaldate reductase; Monodehydroascorbate reductase; Mycothione reductase; N-hydroxy-2-acetamidofluorene reductase; N-acetyl-gamma-glutamyl-phosphate reductase; NADPH—cytochrome-c2 reductase; NADPH—hemoprotein reductase; NADPH:quinone reductase; Nitrite reductase; Nitroquinoline-N-oxide reductase; Orotate reductase; Oxaloglycolate reductase; P-benzoquinone reductase; Phloroglucinol reductase; Precorrin-6A reductase; Progesterone 5α-reductase; Prostaglandin-E2 9-reductase; Protein-disulfide reductase; Protochlorophyllide reductase; Pteridine reductase; Pyrroline-2-carboxylate reductase; Pyrroline-5-carboxylate reductase; Rubredoxin—NAD⁺ reductase; Rubredoxin—NAD(P)⁺ reductase; Salutaridine reductase; Sepiapterin reductase; Sorbose reductase; Sulcatone reductase; Tagaturonate reductase; Tetrahydroxynaphthalene reductase; Trans-2-enoyl-CoA reductase; Trimethylamine-N-oxide reductase; Tropinone reductase; Trypanothione-disulfide reductase; Vomilenine reductase; Xanthommatin reductase; and Zeatin reductase.

In an embodiment, the NADH-dependent or NADPH-dependent enzyme is an oxygenase. In an embodiment, the oxygenase is selected from the group consisting of (S)-limonene 3-monooxygenase; (S)-limonene 7-monooxygenase; 2-hydroxybiphenyl 3-monooxygenase; 2-hydroxycyclohexanone 2-monooxygenase; 2-hydroxyquinoline 8-monooxygenase; 2-nitrophenol 2-monooxygenase; 2,4-dichlorophenol 6-monooxygenase; 2,6-dihydroxypyridine 3-monooxygenase; 3-hydroxybenzoate 4-monooxygenase; 3-hydroxybenzoate 6-monooxygenase; 3,9-dihydroxypterocarpan 6a-monooxygenase; 4-aminobenzoate 1-monooxygenase; 4-hydroxyacetophenone monooxygenase; 4-hydroxybenzoate 3-monooxygenase; 4-hydroxyphenylacetaldehyde oxime monooxygenase; 4-hydroxyphenylacetate 1-monooxygenase; 4-hydroxyquinoline 3-monooxygenase; 4-nitrophenol 2-monooxygenase; 5-O-(4-coumaroyl)-D-quinate 3′-monooxygenase; 27-hydroxycholesterol 7α-monooxygenase; Albendazole monooxygenase; Alkene monooxygenase; Anhydrotetracycline monooxygenase; Anthranilate 3-monooxygenase; Anthraniloyl-CoA monooxygenase; Benzoate 4-monooxygenase; Benzoyl-CoA 3-monooxygenase; Cholestanetriol 26-monooxygenase; Cholesterol 7α-monooxygenase; Cyclopentanone monooxygenase; Dihydrochelirubine 12-monooxygenase; Dihydrosanguinarine 10-monooxygenase; Flavonoid 3′-monooxygenase; Hydroxyphenylacetonitrile 2-monooxygenase; Imidazoleacetate 4-monooxygenase; Kynurenine 3-monooxygenase; L-lysine 6-monooxygenase; Leukotriene-B4 20-monooxygenase; Leukotriene-E4 20-monooxygenase; Limonene 6-monooxygenase; Melilotate 3-monooxygenase; Methyltetrahydroprotoberberine 14-monooxygenase; N-methylcoclaurine 3′-monooxygenase; Orcinol 2-monooxygenase; Phenol 2-monooxygenase; Phenylacetone monooxygenase; Phosphatidylcholine 12-monooxygenase; Protopine 6-monooxygenase; Questin monooxygenase; Quinine 3-monooxygenase; Salicylate 1-monooxygenase; Taxifolin 8-monooxygenase; Trans-cinnamate 2-monooxygenase; Trans-cinnamate 4-monooxygenase; Vanillate monooxygenase; 2-aminobenzenesulfonate 2,3-dioxygenase; 2-chlorobenzoate 1,2-dioxygenase; 2-hydroxyquinoline 5,6-dioxygenase; 3-hydroxy-2-methylpyridinecarboxylate dioxygenase; 3-phenylpropanoate dioxygenase; 4-chlorophenylacetate 3,4-dioxygenase; 4-sulfobenzoate 3,4-dioxygenase; 5-pyridoxate dioxygenase; Anthranilate 1,2-dioxygenase; Benzene 1,2-dioxygenase; Benzoate 1,2-dioxygenase; Biphenyl 2,3-dioxygenase; Naphthalene 1,2-dioxygenase; Nitric oxide dioxygenase; Phthalate 4,5-dioxygenase; Senecionine N-oxygenase; Terephthalate 1,2-dioxygenase; and Toluene dioxygenase.

In an embodiment, the NADH-dependent or NADPH-dependent enzyme is a synthase. In an embodiment, the synthase is selected from the group consisting of (S)-canadine synthase; (S)-cheilanthifoline synthase; (S)-stylopine synthase; 6-methylsalicylic-acid synthase; 6′-deoxychalcone synthase; Berbamunine synthase; Corydaline synthase; Fatty acid synthase; Fatty-acyl-CoA synthase; GDP-L-fucose synthase; Glutamate synthase; Glyceollin synthase; Glycine cleavage system (glycine synthase); Icosanoyl-CoA synthase; Licodione synthase; Lovastatin nonaketide synthase; Mycocerosate synthase; N5-(carboxyethyl)ornithine synthase; Precorrin-3B synthase; PreQI synthase; Prostaglandin-F synthase; Psoralen synthase; Pterocarpin synthase; Salutaridine synthase; and Secologanin synthase.

In an embodiment, the NADH-dependent or NADPH-dependent enzyme is a hydroxylase. In an embodiment, the hydroxylase is selected from the group consisting of 3-hydroxyphenylacetate 6-hydroxylase; 4-hydroxybenzoate 1-hydroxylase; 4′-methoxyisoflavone 2′-hydroxylase; 5-β-cholestane-3α,7α-diol 12α-hydroxylase; 7-deoxyloganin 7-hydroxylase; 7-α-hydroxycholest-4-en-3-one 12α-hydroxylase; 8-dimethylallylnaringenin 2′-hydroxylase; 24-hydroxycholesterol 7α-hydroxylase; Cholesterol 24-hydroxylase; Deoxysarpagine hydroxylase; Isoflavone 2′-hydroxylase; Isoflavone 3′-hydroxylase; Lithocholate 60-hydroxylase; Tabersonine 16-hydroxylase; Taxane 10-β-hydroxylase; Taxane 13-α-hydroxylase; Vinorine hydroxylase.

In an embodiment, the NADH-dependent or NADPH-dependent enzyme is selected from the group consisting of 15-oxoprostaglandin 13-oxidase; Lathosterol oxidase; NADH peroxidase; NADPH peroxidase; 3α,7α,12α-trihydroxycholestan-26-al26-oxidoreductase; Myristoyl-CoA 11 desaturase; Phosphatidylcholine desaturase; ATP-dependent NAD(P)H-hydrate dehydratase; GDP-mannose 4,6-dehydratase; Ketol-acid reductoisomerase; Monoprenyl isoflavone epoxidase; and Sterol 14-demethylase.

### Kits

In another aspect, the present disclosure provides a kit for analyte concentration measurements. In an embodiment, the kit comprises a transducer, such as a nanoparticle transducer, comprising a chromophore; and an enzyme physically associated with the nanoparticle and configured to catalyze a reaction comprising a plurality of reaction elements. In an embodiment, the enzyme is physically associated with the transducer and/or chromophore, such as when the enzymes and transducer and/or enzymes and chromophore are dispersed in a common solvent, coupled to a common substrate, coupled together, encapsulated together in a hydrogel bead, and the like. In an embodiment, the transducer is a transducer according to any of the transducers described herein. In an embodiment, the nanoparticle comprises a Pdot. In an embodiment, the enzyme is an enzyme as described herein. In an embodiment, the kit includes components suitable for performing one or more reactions according to the methods of the present disclosure.

In an embodiment, the plurality of reaction elements comprises one or more reactants including the analyte and one or more products, and wherein an amount of fluorescence emitted from the chromophore is determined by a concentration of a reaction element of the plurality of reaction elements. In an embodiment, the enzyme is an NADH-dependent or NADPH-dependent enzyme, such as discussed further herein with respect to the transducers of the present disclosure. In an embodiment, the NADH-dependent or NADPH-dependent enzyme is selected from the group consisting of a dehydrogenase, a reductase, an oxygenase, a synthase, a hydroxylase, and combinations thereof. In an embodiment, a reaction element of the plurality of reaction elements comprises NADH and/or NAD⁺, and wherein the amount of fluorescence emitted from the chromophore is determined by a concentration of the NADH and/or NAD⁺. In an embodiment, the analyte comprises NADH and/or NAD⁺. In an embodiment, a reaction element of the plurality of reaction elements comprises NADPH and/or NADP⁺, and wherein the amount of fluorescence emitted from the chromophore is determined by a concentration of the NADPH and/or NADP⁺. In an embodiment, the analyte comprises NADPH and/or NADP⁺.

While NADH and NADH-dependent or NADPH and NADPH-dependent enzyme are described, it will be understood that other analyte and enzyme pairs are within the scope of the disclosure. In that regard, in an embodiment, the analyte is glucose and the enzyme is a glucose oxidase. In an embodiment, a reaction element of the plurality of reaction elements comprises oxygen and wherein the amount of fluorescence emitted from the chromophore is determined by a concentration of the oxygen.

As above, the enzyme and the nanoparticle are physically associated. In some cases, the chromophore can be directly mixed with the enzyme for measurements. In some cases, covalent conjugation is provided to link the nanoparticle to the enzyme, yielding a compact probe that can be used for, for example, intracellular sensing. In an embodiment, the enzyme is physically associated with the chromophore. As discussed further herein, such physical association can include an enzyme dispersed in a common solvent with a chromophore, an enzyme coupled to a common substrate with a chromophore, an enzyme lyophilized in a common powder with a chromophore, an enzyme encapsulated with a chromophore in a hydrogel bead, or an enzyme otherwise in physical/chemical contact with a chromophore. In an embodiment, the enzyme physically associated with the chromophore is coupled to the chromophore. In this regard, the enzyme is directly or indirectly physically connected to the chromophore. Such coupling can include a covalent bond. In another embodiment, the coupling between an enzyme and a chromophore is through one or more non-covalent bonds or interactions, such as an ionic bond, van der Waals forces, hydrogen bonding, and the like. Accordingly, in an embodiment, the coupling does not include a covalent bond.

As discussed elsewhere herein, the chromophores can take a number of different forms and comprise a number of different types of chromophores. In an embodiment, the chromophore is the form of a nanoparticle. In an embodiment, the chromophore is a semiconducting polymer, such as in the form of a semiconducting polymer nanoparticle. As described elsewhere herein, in an embodiment, the chromophore is part of a polymer dot or pdot, such as where the chromophore and other polymeric components are present in a condensed, stable, submicron state. In an embodiment, the chromophore can include a chromophoric polymer, such as a semiconducting chromophoric polymer, in an uncondensed state.

As discussed further herein, it has been surprisingly found that the transducers of the present disclosure can operate to detect or monitor analyte concentration through physical association, rather than only through covalent bonding. Accordingly, such physical association can include covalent bonding, wherein the enzyme is covalently bonded to the nanoparticle. Additionally, such physical association include other forms of association, such as through non-covalent bonding of the enzyme and the nanoparticle. Accordingly, in an embodiment, the enzyme is not covalently bonded to the nanoparticle. In an embodiment, the enzyme and the nanoparticle are associated through ionic bonding, van der Waals forces, hydrogen bonding, and the like.

In an embodiment, the enzyme and the nanoparticle are mixed together. In an embodiment, the enzyme and the nanoparticle are encapsulated together in a hydrogel bead. In an embodiment, the enzyme and the nanoparticle are in a lyophilized powder. Such a lyophilized powder can be, for example, rehydrated and/or reconstituted into a common solvent. In an embodiment, the enzyme and the nanoparticle are dispersed in a common solvent. In an embodiment, the enzyme and the nanoparticle are covalently or non-covalently attached to a substrate or surface. In an embodiment, the enzyme and the nanoparticle are covalently or non-covalently attached to a common substrate or surface.

### Methods

In another aspect, the present disclosure provides methods for measuring a concentration of an analyte in a fluid. In an embodiment, the method is performed, in part or in whole, using a system of the present disclosure.

In an embodiment, the method includes contacting the fluid with a transducer according the present disclosure. In an embodiment, the method includes contacting the fluid with a Pdot including a chromophore and an NADH-dependent or NADPH-dependent enzyme coupled to the Pdot configured to catalyze a reaction. In an embodiment, the method includes contacting the fluid with a Pdot including a chromophore and an enzyme physically associated with the Pdot configured to catalyze a reaction. In an embodiment, the method includes contacting the fluid with a chromophore including a semiconducting chromophoric polymer and an enzyme physically associated with the chromophore configured to catalyze a reaction. In an embodiment, the fluid is a liquid. In an embodiment, the fluid is a gas. In an embodiment, the fluid is a combination of a liquid and a gas. While methods including contacting a fluid with, for example, a transducer are described, it will be understood that, in certain embodiments, the methods of the present disclosure comprise contacting the transducers of the present disclosure with a solid or a slurry. In an embodiment, the method includes contacting a transducer as described herein with a biological specimen of TABLE 1.

The chromophore and enzyme can be coupled and/or physically associated as described further herein with respect to the transducers of the present disclosure. Accordingly, in an embodiment, the chromophore and the enzyme are coupled to a substrate. In an embodiment, the chromophore and the enzyme are coupled to a common substrate or to the same surface. Likewise, in an embodiment, the chromophore and the enzyme are dispersed in a common solvent. In an embodiment, the chromophore and the enzyme are encapsulated together in a hydrogel bead. In an embodiment, the enzyme is not coupled to the Pdot, such as wherein the enzyme is not covalently bound to the Pdot. In an embodiment, the Pdot is covalently bound to the enzyme.

In some cases, the chromophore can be directly mixed with the enzyme for measurements. In some cases, covalent conjugation is provided to link the nanoparticle to the enzyme, yielding a compact probe that can be used for, for example, intracellular sensing. In an embodiment, the enzyme is physically associated with the chromophore. As discussed further herein, such physical association can include an enzyme dispersed in a common solvent with a chromophore, an enzyme coupled to a common substrate with a chromophore, an enzyme lyophilized in a common powder with a chromophore, an enzyme encapsulated with a chromophore in a hydrogel bead, or an enzyme otherwise in physical/chemical contact with a chromophore. In an embodiment, the enzyme physically associated with the chromophore is coupled to the chromophore. In this regard, the enzyme is directly or indirectly physically connected to the chromophore. Such coupling can include a covalent bond. In another embodiment, the coupling between an enzyme and a chromophore is through one or more non-covalent bonds or interactions, such as an ionic bond, van der Waals forces, hydrogen bonding, and the like. Accordingly, in an embodiment, the coupling does not include a covalent bond.

As discussed elsewhere herein, the chromophores can take a number of different forms and comprise a number of different types of chromophores. In an embodiment, the chromophore is the form of a nanoparticle. In an embodiment, the chromophore is a semiconducting polymer, such as in the form of a semiconducting polymer nanoparticle. As described elsewhere herein, in an embodiment, the chromophore is part of a polymer dot or pdot, such as where the chromophore and other polymeric components are present in a condensed, stable, submicron state. In an embodiment, the chromophore can include a chromophoric polymer, such as a semiconducting chromophoric polymer, in an uncondensed state.

As discussed further herein, in an embodiment, the reaction comprises a plurality of reaction elements, such as wherein the plurality of reaction elements comprises one or more reactants including the analyte and one or more products. As also discussed further herein, in an embodiment, an amount of fluorescence emitted from the chromophore is determined by a concentration of a reaction element of the plurality of reaction elements.

In an embodiment, the method further includes illuminating the chromophore to induce fluorescence therefrom. In an embodiment, such illumination includes illuminating the chromophore with a wavelength absorbed by the chromophore. In an embodiment, the method further comprises illuminating a second chromophore, such as in a wavelength range different from the light used to illuminate the chromophore, such as for excitation multiplexing. In an embodiment, illumination is provided by an illumination source of a system in accordance with an embodiment of the disclosure.

In an embodiment, the method further includes measuring the fluorescence from the chromophore. In an embodiment, the fluorescence emitted from the chromophore defines a fluorescence ratio equal to a ratio of an amount of fluorescence emitted at a signal fluorescence wavelength to an amount of fluorescence emitted at a control fluorescence wavelength. In an embodiment, the fluorescence ratio is determined by the concentration of the fluid component or fluid constituent.

In an embodiment, the method further comprises determining the concentration of the analyte based on the measured fluorescence. In an embodiment, the determining of the concentration of the analyte comprises measuring fluorescence at the signal fluorescence wavelength and the control fluorescence wavelength; and determining a measured fluorescence ratio based on the measuring and determining a concentration of the analyte based on the measured fluorescence ratio.

As above, the methods include contacting a fluid with a transducer according the present disclosure. Such a fluid can include any fluid suitable for assaying for an analyte. In an embodiment, the fluid is a biological fluid thought or suspected to contain an analyte. In an embodiment, the fluid is selected from blood, plasma, serum, lymph, saliva, tears, interstitial fluid, spinal fluid, urine, sweat, and combinations thereof.

The methods of the present disclosure are suitable to determine or monitor concentrations of a number of analytes in fluids. Such analytes can be those which are consumed or altered by an NADH-dependent or NADPH-dependent enzyme or other enzyme described herein. In an embodiment, the analyte is an amino acid. In an embodiment, the analyte is NADH. In an embodiment, the analyte is selected from the group consisting of ascorbic acid, glutamate, dopamine, cholesterol, alcohol. In an embodiment, the analyte is a drug. In an embodiment, the analyte is a drug metabolite. In an embodiment, the analyte is a protein, a nucleic acid molecule, or a transmitter molecule. In an embodiment, the analyte is a carbohydrate, a lipid, or a metabolite. In an embodiment, the analyte is a sugar. In an embodiment, the analyte is a metabolite. In an embodiment, the metabolite is selected from the group consisting of lactate, glutamate, glucose, and β-hydroxybutyrate. In an embodiment, the metabolite is a metabolite according to any one or more of the metabolites of TABLE 1.

TABLE 1
LIST OF POSSIBLE METABOLITES

| Analytes | Biospecimen |
|---|---|
| Acetaldehyde | Blood, Cerebrospinal fluid (CSF), |
| | Feces, Saliva, Sweat, Urine |
| Acetoacetic acid | Blood, Cytoplasm, CSF, Feces, Saliva, |
| | Urine |
| Adipate semialdehyde | |
| Alanine | Blood, Breast milk, CSF, Feces, Saliva, |
| | Sweat, Urine |
| Alanopine | |
| Albendazole | Blood, Urine |
| Allyl alcohol | |
| Allysine | Blood, Urine |
| α-Ketoisovaleric acid | Blood, CSF, Feces, Saliva, Urine |
| Altruronic acid | |
| Ammonia | Blood, Cytoplasm, CSF, Urine |
| Androstan-3α,17β-diol | |
| Androsterone | Blood, CSF, Urine |
| Apiose | |
| Aquacobalamin | Blood |
| Arabinose | Blood, CSF, Feces, Urine |
| Arabitol | Blood, CSF, Feces, Saliva, Urine |
| Arachidyl alcohol | Saliva |
| Arginine | Blood, CSF, Feces, Saliva, Sweat, Urine |
| Aromadendrin | |
| Ascorbic acid | Amniotic fluid, Blood, Breast milk, |
| | Cytoplasm, CSF, Saliva, Urine |
| Asparagusic acid | |
| Aspartate-semialdehyde | |
| Aspartic acid | Blood, Breast milk, CSF, Feces, Saliva, |
| | Sweat, Urine |
| Benzaldehyde | Blood, Feces, Saliva |
| Benzene | Blood, Breath, Feces, Saliva |
| Benzoyl-CoA | |
| Benzyl alcohol | Blood, Breath, Feces, Saliva |
| Berberine | Blood |
| Betaine aldehyde | |
| Bilirubin | Bile, Blood, CSF, Feces, Urine |
| Biochanin A | Blood, Urine |
| Biphenyl | Breath, |
| Borneol | Saliva, |
| Butanal | Feces, |
| Carnitine | Blood, Breast milk, CSF, Feces, Saliva, |
| | Urine |
| Carveol | |
| Chlordecone | |
| Cholestenone | Blood, Feces |
| Cholesterol | Bile, Blood, CSF, Feces, Saliva, Urine |
| Cholic acid | Bile, Blood, Feces, Urine |
| Chtriol | |
| Cinnamyl alcohol | |
| Cob(I)alamin | Blood |
| Cob(II)alamin | |
| Codeine | Blood, Feces |
| Coniferaldehyde | |
| Coniferyl alcohol | |
| Cortisone | Blood, Saliva, Urine |
| Cucurbitacin | |
| Currayangine | |
| Cyanocobalamin | Blood, CSF, Urine |
| Cyclohexanol | Feces |
| Cyclopentanone | |
| Cysteine | Blood, CSF, Feces, Saliva, Sweat, Urine |
| Cystine | Blood, CSF, Feces, Urine |
| Diaminopimelic acid | Blood, Feces, Urine |
| Diethyl oxalpropionate | |
| Dihydroasparagusic acid | |
| Dihydroorotic acid | Feces |
| Dihydrouracil | Blood, CSF, Feces, Saliva, Urine |
| Dihydroxyacetone | |
| Phosphate Acyl Ester | |
| Dimethylmalonic acid | Feces |
| dTDP-D-galactose | |
| Dyspropterin | |
| Eicosanoyl-CoA | |
| Ephedrine | Blood, Urine |
| Erythrose | Blood, Feces |
| Erythrose 4-phosphate | Cytoplasm |
| Erythrulose | |
| Estradiol | Blood, CSF, Saliva, Urine |
| Estriol | Amniotic fluid, Blood, Urine |
| Ethanol | Blood, CSF, Feces, Saliva, Sweat, Urine |
| Farnesol | Blood |
| Fe2+ | Blood, CSF, Saliva, Urine |
| FMNH2 | |
| Folinic acid | Blood |
| Formaldehyde | Blood, Urine |
| Formic acid | Blood, Breast milk, CSF, Feces, Saliva, |
| | Sweat, Urine |
| Fructose | Blood, CSF, Feces, Saliva, Urine |
| Fumaric acid | Blood, Breast milk, CSF, Feces, Saliva, |
| | Sweat, Urine |
| Galactitol | Amniotic fluid, Blood, CSF, Feces, |
| | Saliva, Urine |
| Galactose | Blood, Breast milk, Cytoplasm, CSF, |
| | Feces, Saliva, Urine |
| Galacturonic acid | Feces, Urine |
| GDP-D-Rhamnose | |
| Geraniol | Feces, Saliva |
| Gluconic acid | Blood, Feces, Saliva, Urine |
| Glucose | Blood, Breast milk, CSF, Feces, |
| | Saliva, Sweat, Urine |
| Glucose 6-phosphate | Blood, Cytoplasm, Feces, Saliva, |
| | Urine |
| Glucuronic acid | Blood, Feces, Urine |
| Glutamic acid | Blood, Breast milk, Feces, Saliva, |
| | Urine |
| Glutamic gamma- | |
| semialdehyde | |
| Glutarate semialdehyde | |
| Glyceraldehyde 3-phosphate | Blood, Cytoplasm |
| Glyceric acid | Blood, CSF, Feces, Saliva, Sweat, |
| | Urine |
| Glycerol | Blood, CSF, Feces, Saliva, Sweat, |
| | Urine |
| Glycerol 3-phosphate | Blood, Feces, Saliva, Urine |
| Glycine | Bile, Blood, CSF, Feces, Saliva, |
| | Sweat, Urine |
| Glycolaldehyde | |
| Glycolic acid | Blood, CSF, Feces, Saliva, Sweat, |
| | Urine |
| Glyoxylic acid | Blood, CSF, Feces, Urine |
| Guanosine diphosphate | |
| mannose | |
| Gulonic acid | Feces |
| Hexadecenal | |
| Histidinol | Prostate tissue |
| Homocitric acid | Feces |
| Homogentisic acid | Blood, Feces, Urine |
| Homoserine | Blood, Feces, Urine |
| Hydrogen | |
| Hydroquinone | Blood, Urine |
| Hydroxylamine | Blood, Urine |
| Hydroxyphenyllactic acid | Blood, CSF, Feces, Saliva, Urine |
| Hydroxypropanedioic acid | |
| Hydroxypropionic acid | Blood, CSF, Feces, Urine |
| Hypotaurine | Blood, Feces, Saliva, Urine |
| Hypoxanthine | Amniotic fluid, Blood, Breast milk, |
| | Cytoplasm, CSF, Feces, Saliva, Urine |
| Iditol | Blood, Feces |
| Imidazoleacetic acid | Blood, CSF, Feces, Urine |
| Indan-1-ol | |
| Indoleacetic acid | Blood, CSF, Feces, Saliva, Urine |
| Inosinic acid | Blood, Cytoplasm, CSF, Feces |
| Isocitric acid | Blood, Cytoplasm, CSF, Feces, Saliva, |
| | Semen, Urine |
| Isopentanol | Feces, Saliva, |
| Isopropyl alcohol | Blood, Breath, CSF, Feces, Saliva, |
| | Urine |
| Kynurenine | Blood, CSF, Feces, Urine |
| Lactaldehyde | |
| Lactic acid | Bile, Blood, Breast milk, Cytoplasm, |
| | CSF, Feces, Saliva, Sweat, Urine |
| Lathosterol | Blood, Feces |
| Leucine | Blood, Breast milk, CSF, Feces, Saliva, |
| | Sweat, Urine |
| Leucoanthocyanidin | |
| Leukotriene B4 | Blood, CSF, Urine |
| Leukotriene E4 | Blood, CSF, Urine |
| Limonene | Blood, Breath, Feces, Saliva, Urine |
| Lithocholic acid | Bile, Blood, Feces, Urine |
| Lysine | Blood, Breast milk, CSF, Feces, Saliva, |
| | Sweat, Urine |
| Lysopine | |
| Malic acid | Blood, Cytoplasm, CSF, Feces, Saliva, |
| | Sweat, Urine |
| Malonic acid | Blood, CSF, Feces, Saliva, Urine |
| Malonic semialdehyde | |
| Mannitol | Blood, CSF, Feces, Saliva, Urine |
| Mannitol 1-phosphate | |
| Mannose 6-phosphate | Feces |
| Menthol | Blood, Feces, Saliva, Urine |
| Mercury | |
| Methoxsalen | Blood, Urine |
| Methyloxirane | |
| Mevalonic acid | Blood, Feces, Saliva, Urine |
| Morphine | Blood, Urine |
| myo-Inositol | Blood, Breast milk, CSF, Feces, Saliva, |
| | Sweat, Urine |
| N,N′-Bis(gamma- | |
| glutamyl)cystine | |
| N2-Succinyl-L-glutamic | |
| acid 5-semialdehyde | |
| N-Acetyl-D-glucosamine | Blood, Breast milk, Feces, Saliva, |
| | Urine |
| N-Acetyl-L-glutamyl | |
| 5-phosphate | |
| N-Acetylmannosamine | Feces, Urine |
| NADH | Blood |
| NADPH | Blood |
| Neomenthol | |
| Nicotinic acid | Blood, CSF, Feces, Saliva, Urine |
| Nitrate | Blood, CSF, Urine |
| N-Methylalanine | Feces |
| N-Methylcoclaurine | |
| Norsanguinarine | |
| Octanol | Feces, Urine |
| O-Phospho-4-hydroxy-L- | |
| threonine | |
| Palmitaldehyde | |
| p-Aminobenzoic acid | Blood, Feces, Urine |
| Pantoic acid | |
| Pectin | Urine |
| Perillyl alcohol | Blood |
| Phenol | Blood, Feces, Saliva, Sweat, Urine |
| Phenylacetaldehyde | |
| Phenylalanine | Blood, Breast milk, CSF, Feces, Saliva, |
| | Sweat, Urine |
| Phenylglyoxylic acid | Urine |
| Pinitol | Feces, Urine |
| p-Menthane-3,8-diol | |
| Prephenate | |
| Progesterone | Blood, CSF |
| Proline | Blood, Breast milk, CSF, Feces, Saliva, |
| | Sweat, Urine |
| Propane-1,2-diol | Saliva |
| Propylene glycol | Blood, Breath, CSF, Feces, Saliva, |
| | Sweat, Urine |
| Prostaglandin E2 | Blood, CSF, Feces, Urine |
| Protocatechuic acid | Blood, Feces, Urine |
| Protochlorophyllide | |
| Protopine | |
| Pyridoxal | Blood, CSF, Feces |
| Pyridoxine | Blood, CSF, Feces, Urine |
| Quinine | Blood, Urine |
| Quinone | Urine |
| Retinal | Blood |
| Retinol | |
| Rhamnose | Feces, |
| Ribitol | Blood, CSF, Feces, Urine |
| Ribose | Blood, CSF, Feces, Saliva, Urine |
| Saccharopine | Blood, Feces, Urine |
| Salicylic acid | Blood, Feces, Saliva, Urine |
| Sepiapterin | CSF |
| Sequoyitol | |
| Serine | Blood, CSF, Feces, Saliva, Sweat, Urine |
| Shikimic acid | Blood, Urine |
| S-Hydroxymethylglutathione | |
| Sorbitol-6-phosphate | Blood |
| Sorbose | Feces, Sweat, Urine |
| Succinic acid | Blood, Breast milk, CSF, Feces, Saliva, |
| | Sweat, Urine |
| Succinic acid semialdehyde | CSF, Urine |
| Sulcatol | |
| Tartaric acid | Feces, Urine |
| Terephthalic acid | Blood, Saliva, Urine |
| Testosterone | Blood, CSF, Saliva, Urine |
| Tetracycline | Blood, Urine |
| Tetradecenoyl-CoA | |
| Tetrahydrodipicolinate | |
| Tetrahydropteridine | |
| Thiomorpholine 3-carboxylate | |
| Threitol | Blood, CSF, Feces, Urine |
| Threonic acid | Blood, Feces, Saliva, Sweat, Urine |
| Threonine | Blood, Breast milk, CSF, Feces, Saliva, |
| | Sweat, Urine |
| Toluene | Blood, Breath, Feces, Saliva, Urine |
| trans-1,2-Dihydrobenzene- | |
| 1,2-diol | |
| trans-Cinnamic acid | Saliva, |
| Trimethylamine | Blood, CSF, Feces, Saliva, Urine |
| Troxerutin | |
| Trypanothione disulfide | |
| Tryptophan | Blood, Breast milk, CSF, Feces, Saliva, |
| | Sweat, Urine |
| Tryptophol | Urine |
| Tyrosine | Blood, Breast milk, CSF, Feces, Saliva, |
| | Sweat, Urine |
| UDP-N-acetylmuraminate | Prostate tissue |
| Ureidoglycolic acid | |
| Uridine diphosphate glucose | Blood, Feces, Urine |
| Uridine diphosphate-N- | Saliva |
| acetylglucosamine | |
| Valine | Blood, Breast milk, CSF, Feces, Saliva, |
| | Sweat, Urine |
| Vanillin | Blood, Feces, Saliva, Urine |
| Vitamin A | Blood, Feces, Saliva, |
| Xanthine | Blood, CSF, Feces, Saliva, Sweat, Urine |
| Xylitol | Blood, CSF, Feces, Saliva, Urine |
| Xylose | Blood, Feces, Saliva, Urine |
| Xylulose | Blood, CSF, Feces, Urine |
| Zeatin | |
| 1,2-Dihydrobenzene-1,2-diol | |
| 1,2-Dihydronaphthalene- | |
| 1,2-diol | |
| 1,3,5-Trihydroxybenzene | Feces |
| 1,5-Anhydrofructose | |
| 1-Amino-propan-2-ol | Saliva |
| 1-Hexadecanol | Feces |
| 1-Piperideine-2-carboxylic | |
| acid | |
| 2,2′-Iminobispropanoic | |
| acid | |
| 2,3-Butanediol | Blood, Feces, Urine |
| 2,4-Dichlorophenol | Blood, Urine |
| 2-Aminobenzoic acid | Blood, CSF, Feces, Urine |
| 2-Aminomuconic acid | |
| semialdehyde | |
| 2′-Deoxymugineic acid | Feces, |
| 2-Exo-hydroxy-1,8-cineole | |
| 2-Hydroxyadipic acid | Blood, Feces, Urine |
| 2-Hydroxybenzaldehyde | Feces |
| 2-Hydroxybutyric acid | Blood, CSF, Feces, Saliva, Sweat, Urine |
| 2′-Hydroxydaidzein | |
| 2-Hydroxystearic acid | Feces |
| 2-Methyl-3-hydroxybutyryl- | |
| CoA | |
| 2-Methyl-3-oxopropanoic acid | |
| 3-(2- | Feces |
| Hydroxyphenyl)propanoic | |
| acid | |
| 3-(3,5-Diiodo-4- | |
| hydroxyphenyl)lactate | |
| 3-(3,5-Diiodo-4- | |
| hydroxyphenyl)pyruvate | |
| 3,5-Diaminohexanoate | |
| 3b,5a,6b-Cholestanetriol | Feces |
| 3-Dehydro-L-gulonate | |
| 3-Dehydrosphinganine | Feces |
| 3-Hydroxybenzoic acid | Blood, Feces, Urine |
| 3-Hydroxybenzyl alcohol | |
| 3-Hydroxybutyric acid | Blood, Cytoplasm, CSF, Feces, Saliva, |
| | Urine |
| 3-Hydroxyisobutyric acid | Blood, CSF, Feces, Saliva, Urine |
| 3-Hydroxypimelyl-CoA | |
| 3-Isopropylmalic acid | |
| 3-O-p-Coumaroylquinic acid | |
| 3-Oxoadipic acid | |
| 3-Oxoglutaric acid | Blood, Urine |
| 3-Phosphoglyceric acid | Blood, Cytoplasm, Saliva, Urine |
| 4-Aminobutyraldehyde | |
| 4-Hydroxybenzaldehyde | Blood, Feces, Urine |
| 4-Hydroxybenzeneacetonitrile | |
| 4-Hydroxybutyric acid | Blood, CSF, Feces, Saliva, Urine |
| 4-Hydroxyphenylacetaldehyde | |
| 4-Hydroxyproline | Blood, CSF, Feces, Saliva, Urine |
| 4-Nitrocatechol | |
| 4-Nitrophenol | |
| 4-Trimethylammoniobutanal | |
| 5,10-Methylene-THF | Blood |
| 6-Hydroxyhexanoic acid | Saliva |
| 6-Phosphogluconic acid | Blood, Cytoplasm, Saliva |
| 7α-Hydroxycholesterol | Blood, CSF, Feces |
| 7-Dehydrocholesterol | Blood, CSF, Urine |
| 9-Hydroxyfluorene | Urine |
| 13,14-Dihydro-15-keto-PGE2 | Blood, Urine |
| 15-HETE | Blood, CSF, Urine |
| 17a-Estradiol | Blood, Urine |
| 17α,20alpha-Dihydroxypregn- | |
| 4-en-3-one | |
| 27-Hydroxycholesterol | Blood, CSF |

In an embodiment, the metabolite is a metabolite according to any one or more of the metabolites of TABLE 2.

TABLE 2
Medically relevant metabolites compatible with the Pdot biosensor.

| | Analyte | Specimen | Medical condition | Enzyme |
|---|---|---|---|---|
| 1 | (−)-Menthol | Urine | Breast cancer | (−)-Menthol |
| | | | | dehydrogenase |
| 2 | 15-HETE | Urine | Zellweger syndrome | 15- |
| | | | | Hydroxyicosatetraenoate |
| | | | | dehydrogenase |
| 3 | 17a-Estradiol | Urine | Glutaryl-CoA | Estradiol 17α- |
| | | | dehydrogenase | dehydrogenase |
| | | | deficiency | |
| 4 | 2,3-Butanediol | Blood | Cirrhosis | Butanediol |
| | | | | dehydrogenase |
| 5 | 2-Hydroxyadipic | Urine | α-aminoadipic aciduria | 2-Oxoadipate reductase |
| | acid | | | |
| 6 | 2- | Urine | Dihydrolipoamide | Lactate dehydrogenase |
| | Hydroxybutyric | | dehydrogenase | |
| | acid | | deficiency | |
| 7 | 3-(2- | Feces | Irritable bowel syndrome | 2-Coumarate reductase |
| | Hydroxyphenyl) | | | |
| | propanoic acid | | | |
| 8 | 3- | Blood | 3-Hydroxyacyl-CoA | β-Hydroxybutyrate |
| | Hydroxybutyric | | dehydrogenase | dehydrogenase |
| | acid | | deficiency | |
| 9 | 3- | Blood | Diabetes mellitus type 2 | 3-Hydroxyisobutyrate |
| | Hydroxyisobutyric | | | dehydrogenase |
| | acid | | | |
| 10 | 4- | Blood | Prostate cancer | 4-Hydroxybenzaldehyde |
| | Hydroxybenz- | | | dehydrogenase |
| | aldehyde | | | |
| 11 | 4- | Blood | Succinic semialdehyde | 4-Hydroxybutyrate |
| | Hydroxybutyric | | dehydrogenase | dehydrogenase |
| | acid | | deficiency | |
| 12 | 4- | Blood | Hydroxyprolinemia | 4-Oxoproline reductase |
| | Hydroxyproline | | | |
| 13 | 7- | Blood | Smith-lemli-opitz | Lathosterol oxidase |
| | Dehydro- | | syndrome | |
| | cholesterol | | | |
| 14 | Acetoacetic acid | Blood | Succinyl CoA: 3- | 2-Oxopropyl-CoM |
| | | | ketoacid CoA transferase | reductase |
| | | | deficiency | |
| 15 | Allysine | Urine | Pyridoxine dependency | L-Aminoadipate- |
| | | | with seizures | semialdehyde |
| | | | | dehydrogenase |
| 16 | a-Ketoisovaleric | Blood | Maple syrup urine | 2-Oxoisovalerate |
| | acid | | disease | dehydrogenase |
| 17 | Ammonia | Blood | Argininosuccinic | Hydroxylamine |
| | | | aciduria | reductase |
| 18 | Androsterone | Blood | Schizophrenia | 3(or 17)α- |
| | | | | Hydroxysteroid |
| | | | | dehydrogenase |
| 19 | Aromatic alcohol | Blood | Uremia | Aryl-Alcohol |
| | | | | dehydrogenase |
| 20 | Ascorbic acid | Blood | Hyperoxalemia | Monodehydroascorbate |
| | | | | reductase |
| 21 | Benzaldehyde | Feces | Ulcerative colitis | Benzaldehyde |
| | | | | dehydrogenase |
| 22 | Bilirubin | Blood | Crigler-Najjar syndrome | Biliverdin reductase |
| | | | Type I | |
| 23 | Butanal | Feces | Campylobacter jejuni | Butanal dehydrogenase |
| | | | infection | |
| 24 | Carnitine | Blood | 3-Hydroxy-3- | Carnitine 3- |
| | | | methylglutaryl-CoA | dehydrogenase |
| | | | synthase deficiency | |
| 25 | Cholesterol | Blood | Gallbladder disease | Cholesterol 24- |
| | | | | hydroxylase |
| 26 | Cholic acid | Blood | Biliary cirrhosis | 7α-Hydroxysteroid |
| | | | | dehydrogenase |
| 27 | Cyclohexanol | Feces | Crohn's disease | Cyclohexanol |
| | | | | dehydrogenase |
| 28 | D-Arabinose | Feces | Colorectal cancer | D-Arabinose 1- |
| | | | | dehydrogenase |
| 29 | D-Arabitol | Blood | Ribose-5-phosphate | D-Arabinitol |
| | | | isomerase deficiency | dehydrogenase |
| 30 | D-Fructose | Blood | Diabetes | Fructose 5- |
| | | | | dehydrogenase |
| 31 | Dihydrouracil | Urine | Dihydropyrimidine | Dihydrouracil |
| | | | dehydrogenase | dehydrogenase |
| | | | deficiency | |
| 32 | D-Malate | Feces | Colorectal cancer | D-malate dehydrogenase |
| 33 | D-Mannitol | Blood | Uremia | Mannitol 2- |
| | | | | dehydrogenase |
| 34 | D-Ribose | Cerebrospinal | Ribose-5-phosphate | Ribose 1-dehydrogenase |
| | | fluid | isomerase deficiency | |
| 35 | D-Threitol | Blood | Ribose-5-phosphate | Erythrulose reductase |
| | | | isomerase deficiency | |
| 36 | D-Xylitol | Blood | Ribose-5-phosphate | D-Xylulose reductase |
| | | | isomerase deficiency | |
| 37 | D-Xylose | Blood | Small intestinal | D-Xylose 1- |
| | | | malabsorption, HIV, & | dehydrogenase |
| | | | diarrhea | |
| 38 | Ethanol | Blood | Drunk driver | Alcohol dehydrogenase |
| 39 | Fe²⁺ | Blood | Parkinson's disease | Ferric-chelate reductase |
| 40 | Formic acid | Blood | Methanol poisoning | Formate dehydrogenase |
| 41 | Galactitol | Blood | Galactosemia | Galactitol 2- |
| | | | | dehydrogenase |
| 42 | Galactose | Blood | Galactose-1-phosphate | Galactose 1- |
| | | | uridyltransferase | dehydrogenase |
| | | | deficiency | |
| 43 | Gluconate | Blood | Schizophrenia | Gluconate 5- |
| | | | | dehydrogenase |
| 44 | Glucose | Blood | Diabetes mellitus | Glucose dehydrogenase |
| 45 | Glutamic acid | Blood | Schizophrenia | Glutamate |
| | | | | dehydrogenase |
| 46 | Glyceric acid | Blood | D-Glyceric acidura | Glycerate |
| | | | | dehydrogenase |
| 47 | Glycerol | Blood | Glycerol kinase | Glycerol dehydrogenase |
| | | | deficiency | |
| 48 | Glycerol-3- | Saliva | Lewy body disease | Glycerol-3-phosphate |
| | phosphate | | | dehydrogenase |
| 49 | Glycine | Blood | D-Glyceric acidemia | Glycine dehydrogenase |
| 50 | Glycolic acid | Blood | Hemodialysis | Glyoxylate reductase |
| 51 | Glyoxylic acid | Urine | Primary hyperoxaluria I | Glyoxylate |
| | | | | dehydrogenase |
| 52 | Homogentisic | Blood | Alkaptonuria | 4-Hydroxyphenylacetate |
| | acid | | | 1-monooxygenase |
| 53 | Hydroxyphenyl- | Cerebrospinal | Morphine treatment | 4-Hydroxyphenyllactate |
| | lactic acid | fluid | | dehydrogenase |
| 54 | Hydroxypropionic | Blood | Gastric cancer | 3-Hydroxypropionate |
| | acid | | | dehydrogenase |
| 55 | Hypotaurine | Urine | Bladder cancer | Hypotaurine |
| | | | | dehydrogenase |
| 56 | Hypoxanthine | Urine | Xanthinuria type 1 | Xanthine dehydrogenase |
| 57 | Indoleacetic acid | Blood | Uremia | Indolelactate |
| | | | | dehydrogenase |
| 58 | Inosinic acid | Blood | Febrile seizures | Inosine-5′- |
| | | | | monophosphate |
| | | | | dehydrogenase |
| 59 | Isocitric acid | Cytoplasm | Anoxia | Isocitrate dehydrogenase |
| 60 | Isopropyl | Urine | Isopropyl alcohol | Isopropanol |
| | alcohol | | poisoning | dehydrogenase |
| 61 | Lactic acid | Blood | Lactic acidosis | Lactate dehydrogenase |
| 62 | L-Alanine | Blood | Pearson Syndrome | Alanine dehydrogenase |
| 63 | L-Arabinose | Cerebrospinal | Ribose-5-phosphate | L-Arabinose 1- |
| | | fluid | isomerase deficiency | dehydrogenase |
| 64 | L-Arabitol | Blood | Ribose-5-phosphate | L-Arabinitol 2- |
| | | | isomerase deficiency | dehydrogenase |
| 65 | L-Arginine | Blood | Argininemia | L-Arginine |
| | | | | dehydrogenase |
| 66 | L-Aspartic acid | Blood | Cirrhosis | Aspartate |
| | | | | dehydrogenase |
| 67 | Lathosterol | Blood | Cerebrotendinous | Lathosterol oxidase |
| | | | xanthomatosis | |
| 68 | L-Cysteine | Blood | Sulfite oxidase | Cystine reductase |
| | | | deficiency | |
| 69 | L-Histidinol | Prostate | Prostate cancer | Histidinol |
| | | tissue | | dehydrogenase |
| 70 | L-Homoserine | Urine | Eosinophilic esophagitis | Homoserine |
| | | | | dehydrogenase |
| 71 | L-Iditol | Feces | Irritable bowel syndrome | L-Iditol 2- |
| | | | | dehydrogenase |
| 72 | Lithocholic acid | Blood | Biliary cirrhosis | 3α-Hydroxycholanate |
| | | | | dehydrogenase |
| 73 | L-Leucine | Blood | Maple syrup urine | Leucine dehydrogenase |
| | | | disease | |
| 74 | L-Lysine | Blood | Pyruvate carboxylase | Lysine dehydrogenase |
| | | | deficiency | |
| 75 | L-Phenylalanine | Blood | Phenylketonuria | Phenylalanine |
| | | | | dehydrogenase |
| 76 | L-Proline | Blood | Hyperprolinemia, type I | Pyrroline-2-carboxylate |
| | | | | reductase |
| 77 | L-Serine | Blood | Phosphoserine | Serine 2-dehydrogenase |
| | | | aminotransferase | |
| | | | deficiency | |
| 78 | L-Threonate | Blood | Colorectal cancer | L-Threonate 3- |
| | | | | dehydrogenase |
| 79 | L-Threonine | Blood | Citrullinemia type II, | L-Threonine 3- |
| | | | neonatal-onset | dehydrogenase |
| 80 | L-Tryptophan | Blood | Schizophrenia | Tryptophan |
| | | | | dehydrogenase |
| 81 | L-Tyrosine | Blood | Tyrosinemia | Phenylalanine |
| | | | | dehydrogenase |
| 82 | L-Valine | Blood | Maple syrup urine | Valine dehydrogenase |
| | | | disease | |
| 83 | L-Xylulose | Cerebrospinal | Leukoencephalopathy | L-Xylulose reductase |
| | | fluid | and peripheral | |
| | | | neuropathy | |
| 84 | Malic acid | Cytoplasm | Anoxia | Malate dehydrogenase |
| 85 | Mercury | Blood | Acute mercury poisoning | Mercury(II) reductase |
| 86 | Mevalonic acid | Blood | Mevalonic aciduria | Mevaldate reductase |
| 87 | myo-Inositol | Blood | Schizophrenia | Inositol 2- |
| | | | | dehydrogenase |
| 88 | N-Acetyl-D- | Urine | Bladder cancer | N-Acetylhexosamine 1- |
| | glucosamine | | | dehydrogenase |
| 89 | Nitrate | Blood | Kidney disease | Nitric oxide |
| | | | | dioxygenase |
| 90 | Octanol | Feces | Celiac disease | Octanol dehydrogenase |
| 91 | Phenylglyoxylic | Urine | Occupational exposure to | Phenylglyoxylate |
| | acid | | styrene | dehydrogenase |
| 92 | Propylene glycol | Blood | Cirrhosis | Lactaldehyde reductase |
| 93 | Prostaglandin E2 | Cerebrospinal | Amyotrophic lateral | 15- |
| | | fluid | sclerosis | Hydroxyprostaglandin |
| | | | | dehydrogenase |
| 94 | Rhamnose | Feces | Colorectal cancer | L-Rhamnose 1- |
| | | | | dehydrogenase |
| 95 | Ribitol | Blood | Ribose-5-phosphate | Ribitol 2-dehydrogenase |
| | | | isomerase deficiency | |
| 96 | Salicylic acid | Blood | Mitochondrial complex I | Salicylate 1- |
| | | | deficiency | monooxygenase |
| 97 | Sorbitol-6- | Blood | Prostate cancer | Sorbitol-6-phosphate 2- |
| | phosphate | | | dehydrogenase |
| 98 | Succinic acid | Blood | Early preeclampsia | Fumarate reductase |
| 99 | Tartaric acid | Urine | Lung cancer | Tartrate dehydrogenase |
| 100 | Testosterone | Blood | Cadmium exposure | 17β-Hydroxysteroid |
| | | | | dehydrogenase |
| 101 | Trimethylamine | Urine | Trimethylaminuria | Trimethylamine-N- |
| | | | | oxide reductase |
| 102 | Uridine | Blood | Rheumatoid arthritis | UDP-glucose 6- |
| | diphosphate | | | dehydrogenase |
| | glucose | | | |
| 103 | Vitamin A | Blood | Hemodialysis | Retinol dehydrogenase |
| 104 | Xanthine | Blood | Xanthinuria | Xanthine dehydrogenase |

Corresponding enzyme-catalyzed reactions

| | |
|---|---|
| 1 | (−)-menthol + NADP⁺ [symbol] (−)-menthone + NADPH + H⁺ |
| 2 | (15S)-15-hydroxy-5,8,11-cis-13-trans-icosatetraenoate + NAD(P)⁺ [symbol] 15-oxo-5,8,11- |
| | cis-13-trans-icosatetraenoate + NAD(P)H + H⁺ |
| 3 | estradiol-17α + NAD(P)⁺ [symbol] estrone + NAD(P)H + H⁺ |
| 4 | butane-2,3-diol + NAD⁺ [symbol] acetoin + NADH + H⁺ |
| 5 | 2-hydroxyadipate + NAD⁺ [symbol] 2-oxoadipate + NADH + H⁺ |
| 6 | 2-hydroxybutanoic acid + NAD⁺ [symbol] 2-Oxobutanoate + NADH + H⁺ |
| 7 | 3-(2-hydroxyphenyl)propanoate + NAD⁺ [symbol] 2-coumarate + NADH + H⁺ |
| 8 | (R)-3-hydroxybutanoate + NAD⁺ [symbol] acetoacetate + NADH + H⁺ |
| 9 | 3-hydroxy-2-methylpropanoate + NAD⁺ [symbol] 2-methyl-3-oxopropanoate + NADH + H⁺ |
| 10 | 4-hydroxybenzaldehyde + NAD⁺ + H₂O [symbol] 4-hydroxybenzoate + NADH + 2 H⁺ |
| 11 | 4-hydroxybutanoate + NAD⁺ [symbol] succinate semialdehyde + NADH + H⁺ |
| 12 | 4-hydroxy-L-proline + NAD⁺ [symbol] 4-oxoproline + NADH + H⁺ |
| 13 | 5α-cholest-7-en-3β-ol + NAD(P)H + H⁺ + O₂ [symbol] cholesta-5,7-dien-3β-ol + |
| | NAD(P)⁺ + 2 H₂O |
| 14 | 2-mercaptoethanesulfonate + acetoacetate + NADP⁺ [symbol] 2-(2- |
| | oxopropylthio)ethanesulfonate + CO₂ + NADPH |
| 15 | L-2-aminoadipate 6-semialdehyde + NAD(P)⁺ + H₂O [symbol] L-2-aminoadipate + |
| | NAD(P)H + H⁺ |
| 16 | 3-methyl-2-oxobutanoate + CoA + NAD⁺ [symbol] 2-methylpropanoyl-CoA + CO₂ + |
| | NADH |
| 17 | NH₃ + NAD⁺ + H₂O [symbol] hydroxylamine + NADH + H⁺ |
| 18 | androsterone + NAD(P)⁺ [symbol] 5α-androstane-3,17-dione + NAD(P)H + H⁺ |
| 19 | an aromatic alcohol + NAD⁺ [symbol] an aromatic aldehyde + NADH + H⁺ |
| 20 | NADH + H⁺ + 2 monodehydroascorbate [symbol] NAD⁺ + 2 ascorbate |
| 21 | benzaldehyde + NAD⁺ + H₂O [symbol] benzoate + NADH + 2 H⁺ |
| 22 | bilirubin + NAD(P)⁺ [symbol] biliverdin + NAD(P)H |
| 23 | butanal + CoA + NAD(P)⁺ [symbol] butanoyl-CoA + NAD(P)H + H⁺ |
| 24 | carnitine + NAD⁺ [symbol] 3-dehydrocarnitine + NADH + H⁺ |
| 25 | cholesterol + NADPH + H+ + O₂ [symbol] (24S)-24-hydroxycholesterol + NADP+ + H₂O |
| 26 | 3α,7α,12α-trihydroxy-5β-cholanate + NAD⁺ [symbol] 3α,12α-dihydroxy-7-oxo-5β- |
| | cholanate + NADH + H⁺ |
| 27 | cyclohexanol + NAD+ [symbol] cyclohexanone + NADH + H⁺ |
| 28 | D-arabinose + NAD(P)⁺ [symbol] D-arabinono-1,4-lactone + NAD(P)H + H⁺ |
| 29 | D-arabinitol + NAD⁺ [symbol] D-ribulose + NADH + H⁺ |
| 30 | D-fructose + acceptor [symbol] 5-dehydro-D-fructose + reduced acceptor |
| 31 | 5,6-dihydrouracil + NAD⁺ [symbol] uracil + NADH + H⁺ |
| 32 | (R)-malate + NAD⁺ [symbol] pyruvate + CO₂ + NADH |
| 33 | D-mannitol + NAD⁺ [symbol] D-fructose + NADH + H⁺ |
| 34 | D-ribose + NADP⁺ + H₂O [symbol] D-ribonate + NADPH + H⁺ |
| 35 | D-threitol + NADP⁺ [symbol] D-erythrulose + NADPH |
| 36 | xylitol + NAD⁺ [symbol] D-xylulose + NADH + H⁺ |
| 37 | D-xylose + NAD⁺ [symbol] D-xylonolactone + NADH + H⁺ |
| 38 | CH₃CH₂OH + NAD⁺ [symbol] CH₃CHO + NADH + H⁺ |
| 39 | 2 Fe(II) + NAD⁺ [symbol] 2 Fe(III) + NADH + H⁺ |
| 40 | formate + NAD⁺ [symbol] CO₂ + NADH + H⁺ |
| 41 | galactitol + NAD⁺ [symbol] D-tagatose + NADH + H⁺ |
| 42 | D-galactose + NAD⁺ [symbol] D-galactono-1,4-lactone + NADH + H⁺ |
| 43 | D-gluconate + NAD(P)⁺ [symbol] 5-dehydro-D-gluconate + NAD(P)H + H⁺ |
| 44 | β-D-glucose + NAD(P)⁺ [symbol] D-glucono-1,5-lactone + NAD(P)H + H⁺ |
| 45 | L-glutamate + H₂O + NAD⁺ [symbol] 2-oxoglutarate + NH₃ + NADH |
| 46 | (D)-glycerate + NAD⁺ [symbol] hydroxypyruvate + NADH + H⁺ |
| 47 | glycerol + NAD⁺ [symbol] glycerone + NADH |
| 48 | sn-glycerol 3-phosphate + NAD(P)⁺ [symbol] glycerone phosphate + NAD(P)H + H⁺ |
| 49 | glycine + H₂O + NAD⁺ [symbol] glyoxylate + NH₃ + NADH + H⁺ |
| 50 | glycolate + NAD⁺ [symbol] glyoxylate + NADH |
| 51 | glyoxylate + CoA + NADP⁺ [symbol] oxalyl-CoA + NADPH + H⁺ |
| 52 | 4-hydroxyphenylacetate + NAD(P)H + H⁺ + O₂ [symbol] homogentisate + NAD(P)⁺ + H₂O |
| 53 | 3-(4-hydroxyphenyl)lactate + NAD(P)⁺ [symbol] 3-(4-hydroxyphenyl)pyruvate + |
| | NAD(P)H + H⁺ |
| 54 | 3-hydroxypropanoate + NAD⁺ [symbol] 3-oxopropanoate + NADH + H⁺ |
| 55 | hypotaurine + H₂O + NAD⁺ [symbol] taurine + NADH + H⁺ |
| 56 | Hypoxanthine + NAD⁺ + H₂O [symbol] urate + NADH |
| 57 | (indol-3-yl)lactate + NAD⁺ [symbol] (indol-3-yl)pyruvate + NADH + H⁺ |
| 58 | inosine 5′-phosphate + NAD⁺ + H₂O [symbol] xanthosine 5′-phosphate + NADH + H⁺ |
| 59 | Isocitrate + NAD(P)⁺ [symbol] 2-oxoglutarate + CO₂ + NAD(P)H |
| 60 | propan-2-ol + NADP⁺ [symbol] acetone + NADPH + H⁺ |
| 61 | lactate + NAD⁺ [symbol] pyruvate + NADH |
| 62 | L-alanine + H₂O + NAD⁺ [symbol] pyruvate + NH₃ + NADH + H⁺ |
| 63 | L-arabinose + NAD⁺ [symbol] L-arabinono-1,4-lactone + NADH + H⁺ |
| 64 | L-arabinitol + NAD⁺ [symbol] L-ribulose + NADH + H⁺ |
| 65 | L-arginine + H₂O + NAD(P)⁺ [symbol] 5-guanidino-2-oxopentanoate + NH₃ + NAD(P)H |
| 66 | L-aspartate + H₂O + NAD(P)⁺ [symbol] oxaloacetate + NH3 + NAD(P)H + H⁺ |
| 67 | 5α-cholest-7-en-3β-ol + NAD(P)H + H⁺ + O₂ [symbol] cholesta-5,7-dien-3β-ol + |
| | NAD(P)⁺ + 2 H₂O |
| 68 | 2 L-cysteine + NAD⁺ [symbol] L-cystine + NADH + H⁺ |
| 69 | L-histidinol + 2 NAD⁺ [symbol] L-histidine + 2 NADH + 2 H⁺ |
| 70 | L-homoserine + NAD(P)⁺ [symbol] L-aspartate 4-semialdehyde + NAD(P)H + H⁺ |
| 71 | L-iditol + NAD⁺ [symbol] L-sorbose + NADH + H⁺ |
| 72 | 3α-hydroxy-5β-cholanate + NAD⁺ [symbol] 3-oxo-5β-cholanate + NADH + H⁺ |
| 73 | L-leucine + H₂O + NAD⁺ [symbol] 4-methyl-2-oxopentanoate + NH₃ + NADH + H⁺ |
| 74 | L-lysine + NAD⁺ [symbol] 1,2-didehydropiperidine-2-carboxylate + NH₃ + NADH + H⁺ |
| 75 | L-phenylalanine + H₂O + NAD⁺ [symbol] phenylpyruvate + NH₃ + NADH + H⁺ |
| 76 | L-proline + NAD(P)⁺ [symbol] 1-pyrroline-2-carboxylate + NAD(P)H + H⁺ |
| 77 | L-serine + H₂O + NAD⁺ [symbol] 3-hydroxypyruvate + NH₃+ NADH + H⁺ |
| 78 | L-threonate + NAD⁺ [symbol] 3-dehydro-L-threonate + NADH + H⁺ |
| 79 | L-threonine + NAD⁺ [symbol] L-2-amino-3-oxobutanoate + NADH + H⁺ |
| 80 | L-tryptophan + NAD(P)⁺ + H₂O [symbol] (indol-3-yl)pyruvate + NH₃ + NAD(P)H + H⁺ |
| 81 | L-tyrosine + H₂O + NAD⁺ [symbol] 3-(4-Hydroxyphenyl)pyruvate + Ammonia + NADH + |
| | H⁺ |
| 82 | L-valine + H₂O + NAD⁺ [symbol] 3-methyl-2-oxobutanoate + NH₃ + NADH + H⁺ |
| 83 | xylitol + NADP⁺ [symbol] L-xylulose + NADPH + H⁺ |
| 84 | (S)-malate + NAD⁺ [symbol] pyruvate + CO₂ + NADH |
| 85 | Hg²⁺ + NADPH → Hg⁰ + H⁺ + NADP⁺ |
| 86 | (R)-mevalonate + NAD⁺ [symbol] mevaldate + NADH + H⁺ |
| 87 | myo-inositol + NAD⁺ [symbol] 2,4,6/3,5-pentahydroxycyclohexanone + NADH + H⁺ |
| 88 | N-acetyl-D-glucosamine + NAD⁺ [symbol] N-acetyl-D-glucosaminate + NADH + H⁺ |
| 89 | 2 nitric oxide + 2 O₂ + NAD(P)H [symbol] 2 nitrate + NAD(P)⁺ + H⁺ |
| 90 | 1-octanol + NAD⁺ [symbol] 1-octanal + NADH + H⁺ |
| 91 | phenylglyoxylate + NAD⁺ + CoA-SH [symbol] benzoyl-S-CoA + CO₂ + NADH |
| 92 | (R)[or (S)]-propane-1,2-diol + NAD⁺ [symbol] (R)[or (S)]-lactaldehyde + NADH + H⁺ |
| 93 | (5Z,13E)-(15S)-11α,15-dihydroxy-9-oxoprost-13-enoate + NAD⁺ [symbol] (5Z,13E)-11α- |
| | hydroxy-9,15-dioxoprost-13-enoate + NADH + H⁺ |
| 94 | L-rhamnofuranose + NAD⁺ [symbol] L-rhamno-1,4-lactone + NADH + H⁺ |
| 95 | ribitol + NAD⁺ [symbol] D-ribulose + NADH + H⁺ |
| 96 | salicylate + NADH + 2 H⁺ + O₂ [symbol] catechol + NAD⁺ + H₂O + CO₂ |
| 97 | D-sorbitol 6-phosphate + NAD⁺ [symbol] D-fructose 6-phosphate + NADH + H⁺ |
| 98 | succinate + NAD⁺ [symbol] fumarate + NADH + H⁺ |
| 99 | tartrate + NAD⁺ [symbol] oxaloglycolate + NADH + H⁺ |
| 100 | testosterone + NAD(P)⁺ [symbol] androst-4-ene-3,17-dione + NAD(P)H |
| 101 | NADH + H⁺ + trimethylamine N-oxide [symbol] NAD⁺ + trimethylamine + H₂O |
| 102 | UDP-glucose + 2 NAD⁺ + H₂O [symbol] UDP-glucuronate + 2 NADH + 2 H⁺ |
| 103 | retinol + NAD⁺ [symbol] retinal + NADH + H⁺ |
| 104 | xanthine + NAD⁺ + H₂O [symbol] urate + NADH + H⁺ |

In some embodiments, the order in which some or all of the steps are described in each process should not be deemed limiting. Rather, one of ordinary skill in the art having the benefit of the present disclosure will understand that some of the steps may be executed in a variety of orders not illustrated, or even in parallel.

### Systems

In another aspect, the present disclosure provides a system for analyte concentration measurements. In an embodiment, the system comprises transducer, such as a nanoparticle transducer and/or a transducer substrate, or a kit as described further herein. In an embodiment, the system is configured for and suitable to perform the methods of the present disclosure.

As discussed elsewhere herein, the transducers and kits of the present disclosure include a chromophore, such as a chromophore physically associated with an enzyme. In some cases, the chromophore can be directly mixed with the enzyme for measurements. In some cases, covalent conjugation is provided to link the nanoparticle to the enzyme, yielding a compact probe that can be used for, for example, intracellular sensing. In an embodiment, the enzyme is physically associated with the chromophore. As discussed further herein, such physical association can include an enzyme dispersed in a common solvent with a chromophore, an enzyme coupled to a common substrate with a chromophore, an enzyme lyophilized in a common powder with a chromophore, an enzyme encapsulated with a chromophore in a hydrogel bead, or an enzyme otherwise in physical/chemical contact with a chromophore. In an embodiment, the enzyme physically associated with the chromophore is coupled to the chromophore. In this regard, the enzyme is directly or indirectly physically connected to the chromophore. Such coupling can include a covalent bond. In another embodiment, the coupling between an enzyme and a chromophore is through one or more non-covalent bonds or interactions, such as an ionic bond, van der Waals forces, hydrogen bonding, and the like. Accordingly, in an embodiment, the coupling does not include a covalent bond.

As discussed elsewhere herein, the chromophores can take a number of different forms and comprise a number of different types of chromophores. In an embodiment, the chromophore is the form of a nanoparticle. In an embodiment, the chromophore is a semiconducting polymer, such as in the form of a semiconducting polymer nanoparticle. As described elsewhere herein, in an embodiment, the chromophore is part of a polymer dot or pdot, such as where the chromophore and other polymeric components are present in a condensed, stable, submicron state. In an embodiment, the chromophore can include a chromophoric polymer, such as a semiconducting chromophoric polymer, in an uncondensed state.

In an embodiment, the system includes an illumination source configured to illuminate the chromophore of the transducer, the transducer substrate, the kit, or the transducer to induce fluorescence therefrom. In an embodiment, the illumination source is a laser. In an embodiment, the illumination source is a laser diode. In an embodiment, the illumination source is a LED (light emitting diode). In an embodiment, the illumination source is a lamp.

In an embodiment, the illumination source is configured to emit electromagnetic radiation configured to excite the chromophore, such as to emit fluorescence therefrom.

In an embodiment, the illumination source is a first illumination source, the system including a second illumination configured to emit second electromagnetic radiation, such as second electromagnetic radiation having a wavelength range different from the electromagnetic radiation emitted from the first illumination source. Such first and second illumination sources may be suitable to excite transducers having different chromophores, such as chromophores configured to absorb and be excited by electromagnetic radiation having different wavelength ranges. In this regard, such systems are suitable for excitation multiplexing, as described further herein.

In an embodiment, the system includes a photodetector configured to generate a signal based on the fluorescence from the chromophore. In an embodiment, the photodetector is a first photodetector and the signal is a first signal and the system includes a second photodetector configured to generate a second signal. In an embodiment, the second photodetector is configured to generate the second signal based on light having a wavelength range different from the first light. In this regard, the system may be configured to generate first and second signals from, for example, fluorescence from different chromophores, such as chromophores that are part of different transducers configured to react with different enzymes. In this regard, the system may be configured to perform emission multiplexing. In this regard, the system also may be configured to generate first and second signals, for example, at a signal wavelength and at a control wavelength. In this regard, the system may be configured to perform ratiometric fluorescence measurements, as described further herein with respect to the methods of the present disclosure.

In an embodiment, the system includes a controller operatively coupled to the illumination source and the photodetector. In embodiment, the controller includes logic, that when executed by the controller, causes the system to perform operations. Such operations can be configured to perform one or more of the methods of the present disclosure. In an embodiment, the operations include illuminating the chromophore with the illumination source; and determining a concentration of the analyte based upon the signal from the photodetector. As discussed further herein with respect to the EXAMPLES of the present disclosure, an amount or intensity of the fluorescence emitted from the transducers of the present disclosure can be based upon a concentration of an analyte. In this regard, an amount or intensity of detected/measured fluorescence of the chromophores of the transducers may be used to infer and/or calculate a concentration of an analyte.

In an embodiment, the photodetectors are configured to detect an amount of signal fluorescence at the signal fluorescence wavelength and an amount of control fluorescence at the control fluorescence wavelength. In an embodiment, the controller includes further logic that, when executed by the controller, causes the system to perform operations including determining a measured fluorescence ratio based on the measured amounts of the signal fluorescence and the control fluorescence. In an embodiment, determining the concentration of the analyte is based on the measured fluorescence ratio. In an embodiment, the system is configured to generate a signal indicative of the concentration of the analyte.

In an embodiment, the system is shaped to receive a transducer substrate as described further herein. In an embodiment, the transducer substrate is configured to receive a sample, such as a fluid sample, containing or potentially containing an analyte for analysis with the system.

In some embodiments, the processes or operations explained above are described in terms of computer software and hardware. The techniques described may constitute machine-executable instructions embodied within a tangible or non-transitory machine (e.g., computer) readable storage medium, that when executed by a machine will cause the machine to perform the operations described.

In an embodiment, the non-transitory, machine-readable storage medium has instructions stored thereon, which when executed by a processing system, cause the processing system to perform operations comprising, for example, steps or portions of the methods of the present disclosure.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.). Additionally, the processes may be embodied within hardware, such as an application specific integrated circuit (“ASIC”) or otherwise.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

### EXAMPLES

### Example 1: Materials

Poly[9,9-dioctylfluorenyl-2,7-diyl]—End capped with Dimethylphenyl (DMP) (PFO, ADS129BE, Mw: 40,000-150,000), Poly[9,9-dihexylfluorenyl-2,7-diyl]—End capped with DMP (PDHF, ADS130BE, Mw: 40,000-150,000), Poly[(9,9-dioctylfluorenyl-2,7-diyl)-alt-co-(1,4-benzo-{2,1′,3}-thiadiazole)] (PFBT, ADS133YE, Mw: 15,000-200,000), Poly[{2-methoxy-5-(2-ethylhexyloxy)-1,4-(1-cyanovinylenephenylene)}-co-{2,5-bis(N,N′-diphenylamino)-1,4-phenylene}] (DPA-CNPPV, ADS113RE, Mw: 15,000-50,000), Poly[{9,9-dihexyl-2,7-bis(1-cyanovinylene)fluorenylene}-alt-co-{2,5-bis(N,N′-diphenylamino)-1,4-phenylene}](DPA-CNPF, ADS111RE, Mw: 25,000-250,000), Poly[2-methoxy-5-(2-ethylhexyloxy)-1,4-phenylene-vinylene]—End capped with Polysilsesquioxane (POSS) (MEHPPV, ADS200RE, Mw: >100,000), Poly[2-methoxy-5-(2-ethylhexyloxy)-1,4-(1-cyanovinylene-1,4-phenylene)] (CNPPV, ADS110RE, Mw: ˜15,000) were obtained from America Dye Source Inc. (Montreal, Canada). L-Phenylalanine dehydrogenase from Sporosarcina sp. (PheDH, 1.4.1.20), poly(styrene-comaleic anhydride) (PSMA, average Mw: ˜1,700), anhydrous tetrahydrofuran (THF, >99.9%) were obtained from Sigma-Aldrich (St. Louis, USA), β-nicotinamide adenine dinucleotide hydrate, oxidized form (NAD⁺) and reduced form (NADH), β-nicotinamide adenine dinucleotide phosphate, oxidized form (NADP⁺) and reduced form (NADPH) were obtained from Tokyo Chemical Industry Co., Ltd. (Tokyo, Japan) without further purification unless otherwise indicated. Poly[(9,9-dioctylfluorenyl-2,7-diyl)-co-(1,4-benzo-{2,1′,3}-thiadiazole)]-co-4,7-bis(thiophen-2-yl)benzo-2,1,3-thiadiazole] (PFBTTBT) and Poly[2,7-(9,9-di-octyl-fluorene)-alt-4,7-bis(thiophen-2-yl)benzo-2,1,3-thiadiazole] (PFTBT were synthesized in our group. Milli-Q water (18.2 MΩ·cm⁻² at 25° C.) was used throughout the experiment, all other chemical reagents were used as received.

### Example 2: Synthesis of NADH-Sensitive Pdots

Pdots were prepared using the nanoprecipitation method. In a typical preparation, the fluorescent conjugated polymers were dissolved in anhydrous THF to make a stock solution (1.0 mg mL⁻¹). The stock solution was further diluted in THF to produce mixture containing of fluorescent polymers (0.1 mg mL⁻¹) and functional polymer PSMA (0.02 mg mL⁻¹). A 5-mL aliquot of the above solution was quickly dispersed into 10 mL of Milli-Q water under vigorous sonication. THF was removed by blowing nitrogen gas at 90° C. for about 60 min. A small fraction of aggregates was removed by filtration through a 0.2 μm membrane filter.

Pdots have been developed with energy band theory that span the full range of the visible spectrum. FIG. 8 presents the chemical structures of the semiconducting polymers (included PFO, PDHF, PFBT, PFBTTBT, PFTBT, DPA-CNPPV, and DPA-CNPF) employed in this work. NADH-sensitive Pdots were prepared via a facile nanoprecipitation method by folding and distorting the polymers through hydrophobic interactions with amphiphilic polymer PSMA.

### Example 3: Characterization of NADH-Sensitive Pdots

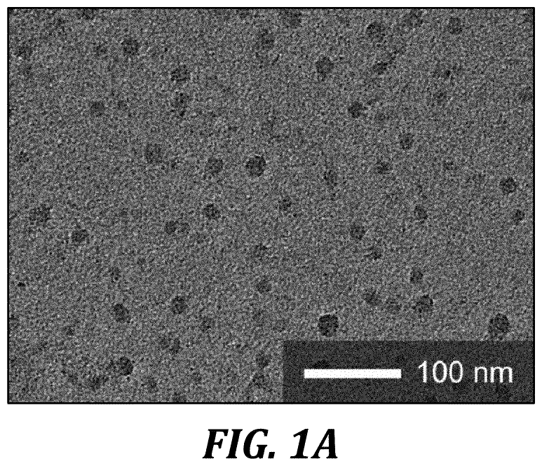

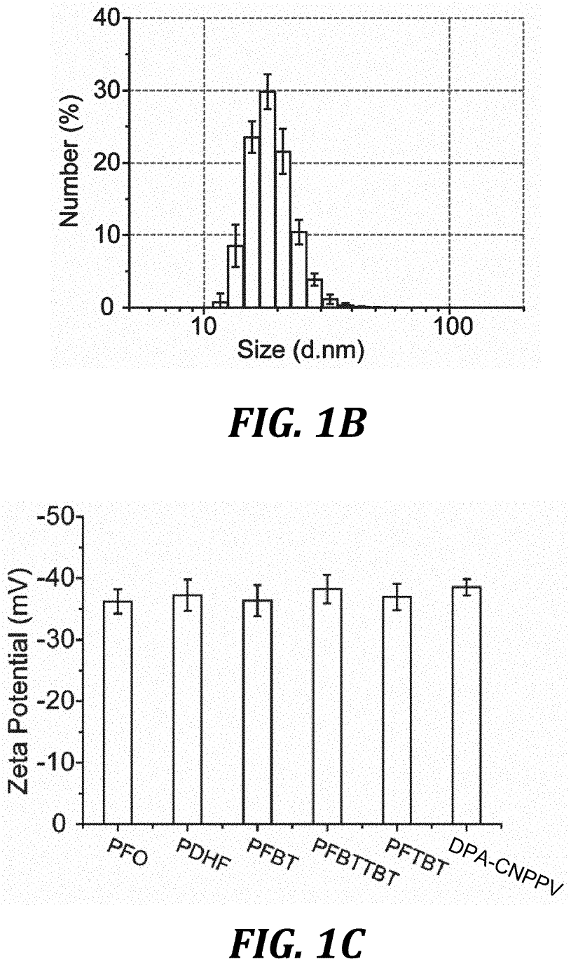

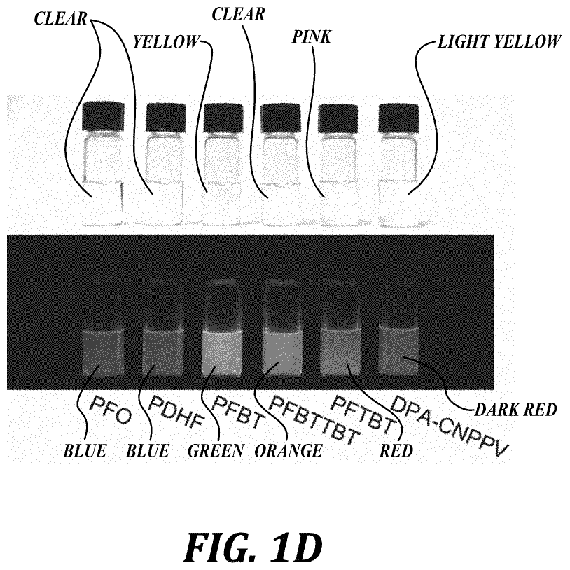

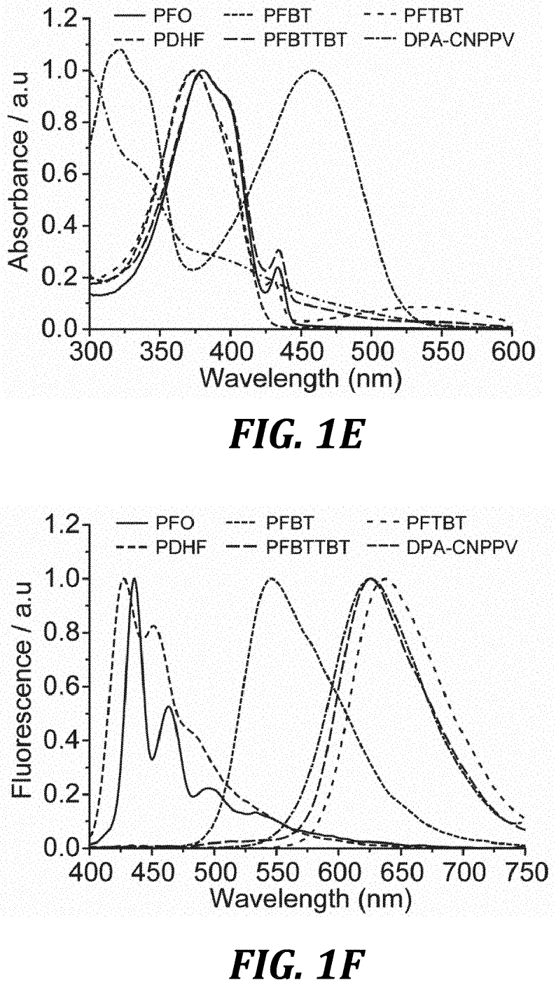

The morphology of obtained Pdots were characterized by transmission electron microscopy (TEM), which shown that Pdots were monodispersed and approximately spherical in shape (FIG. 1A). The average diameter of Pdots determined by dynamic light scattering (DLS) with a hydrodynamic diameter of −19 nm (FIG. 1B). Zeta potential measurements showed that Pdots had a negatively charged surface at neutral pH, and an initial ζ-potential of approximately −37 mV (FIG. 1C). These clear Pdots remained stable in phosphate-buffered saline (PBS) solution for weeks at room temperature (FIG. 9) with no obvious sign of further aggregation or decomposition. FIG. 1D shows the photographs of Pdot suspensions under white light and 365 nm ultraviolet (UV) light, respectively (from left to right: PFO, PDHF, PFBT, DPA-CNPPV, PFBTTBT, and PFTBT Pdots). The changes in absorption and fluorescence spectra vary depending on the polymer structure as indicated in FIG. 1E. Upon excitation, the aqueous Pdots suspensions exhibited strong fluorescence with a nearly full color emission (400-750 nm) (FIG. 1F).

UV-Vis absorption spectra were recorded on a DU 720 scanning spectrophotometer (Beckman Coulter, Inc., CA, USA). Fluorescence spectra were obtained and calibrated using a LS-55 fluorescence spectrometer (LS55, PerkinElmer Life and Analytical Sciences, Shelton, Conn., USA). Fluorescence quantum yields were measured using a Hamamatsu photonic multichannel analyzer C10027 equipped with a CCD camera and an integrating sphere. For the quantum-yield calibration, we used the solvent as the reference. Size distribution and zeta potential of Pdots in aqueous solution were determined by dynamic light scattering (DLS) with a Malvern Zetasizer Nano ZS instrument. The Pdots morphology was recorded on a FEI Tecnai F20 TEM operating at 200 kV.

The size and photophysical properties of Pdots were summarized in TABLE 3.

TABLE 3
SUMMARY OF SIZE AND PHOTOPHYSICAL
PROPERTIES OF PDOTS.

| | λₘₐₓ^{abs} | λₘₐₓ^{em} | size | ζ | LOD | |
|---|---|---|---|---|---|---|
| Pdots | (nm) ^{a} | (nm) ^{b} | (nm) | (mV) ^{c} | (μM) ^{d} | K_{SV} (M⁻¹) ^{e} |
| PFO | 380 | 436 | 20.2 | −36.2 | 25 | 1.04 × 10³ |
| PDHF | 374 | 428 | 22.1 | −37.2 | 36 | 0.94 × 10³ |
| PFBT | 322, 458 | 546 | 19.3 | −36.4 | 14 | 4.89 × 10³ |
| PFBTTBT | 380 | 626 | 19.7 | −38.2 | 27 | 1.17 × 10³ |
| PFTBT | 374, 528 | 638 | 17.5 | −36.9 | 28 | 0.97 × 10³ |
| DPA- | 294 | 627 | 18.8 | −38.5 | 3.1 | 2.89 × 10^{5 f} |
| CNPPV | | | | | | |

^{a} Absorption maximum.
^{b} Fluorescence maximum.
^{c} Zeta potential.
^{d} Limit of detection.
^{e} Quenching constant (0-2 mM).
^{f} Sensitivity (0-2 mM).

### Example 4: Fluorescence Response of Pdots to NADH

The fluorescence responses of Pdots toward NADH were first investigated in aqueous solution to find the best candidate. The present Example demonstrates pdots dispersed in a common solvent with NADH.

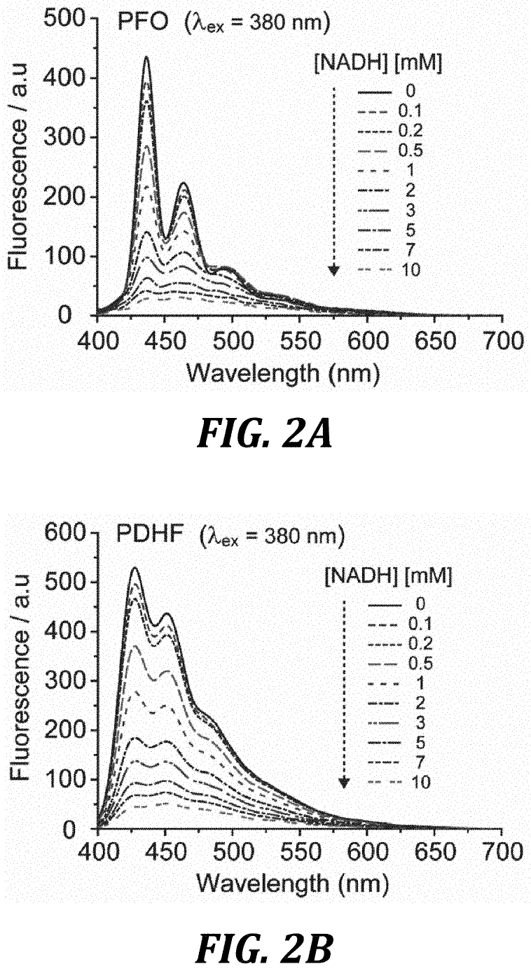

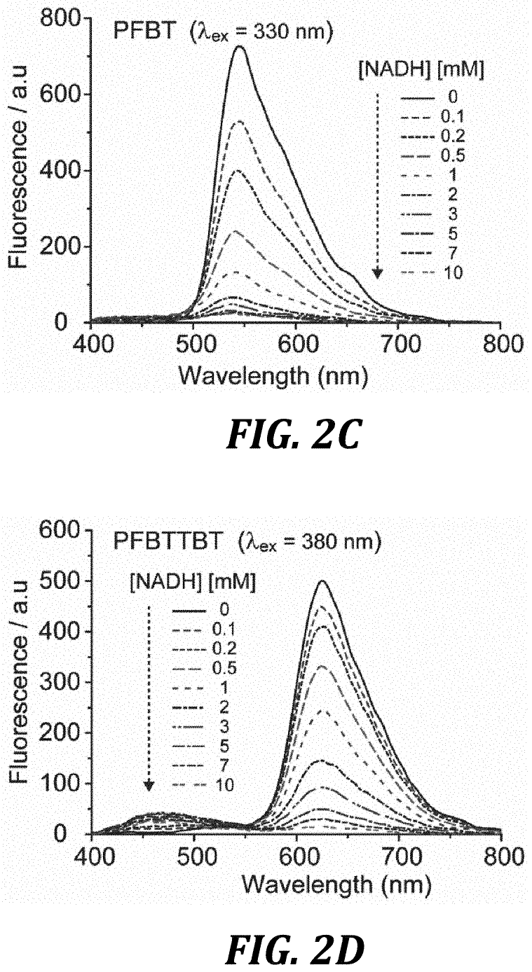

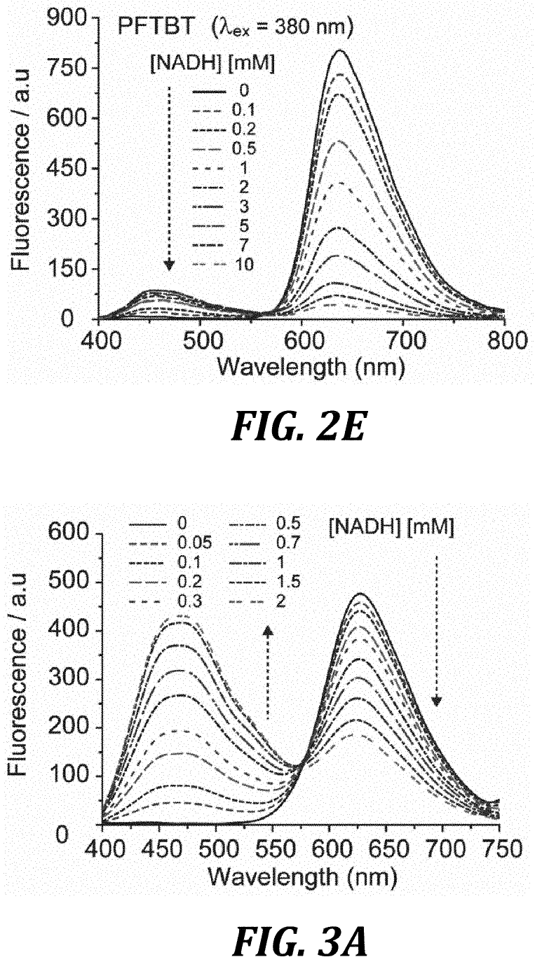

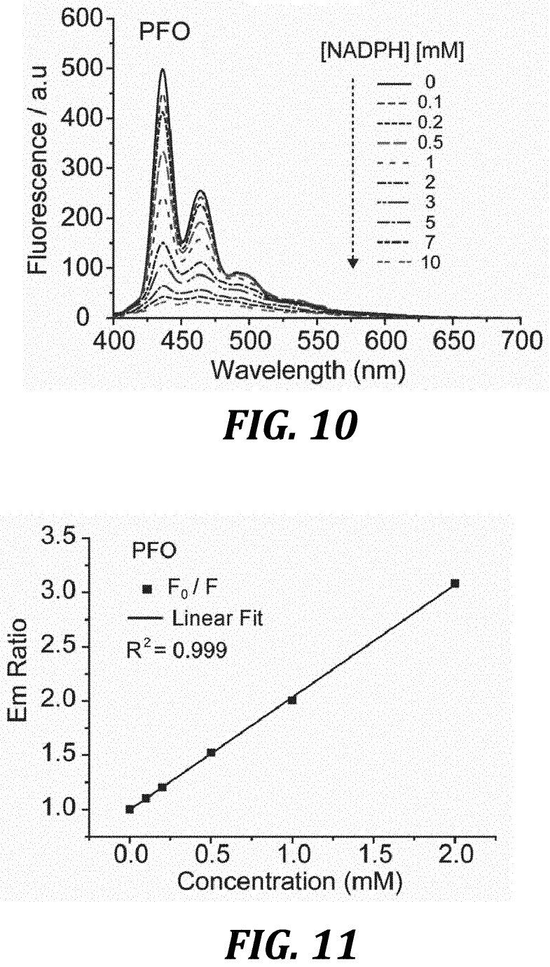

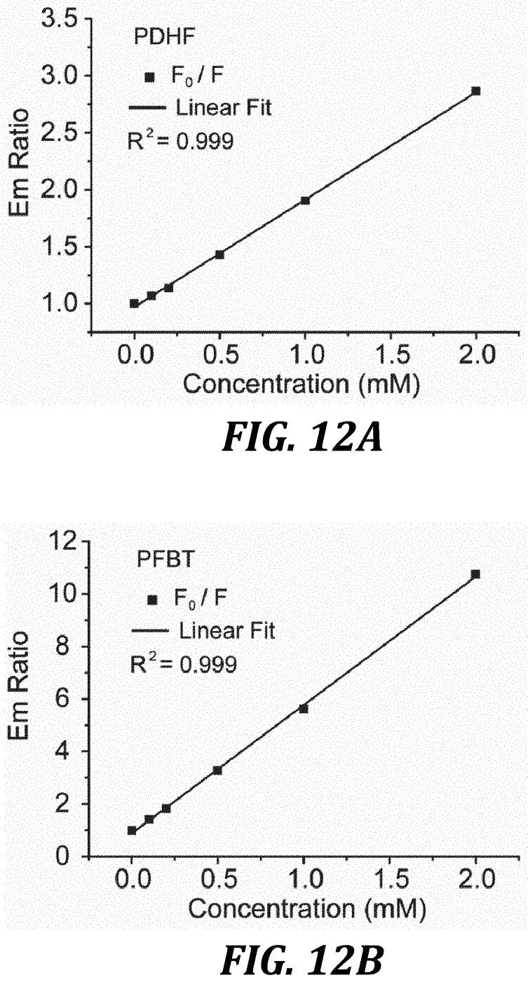

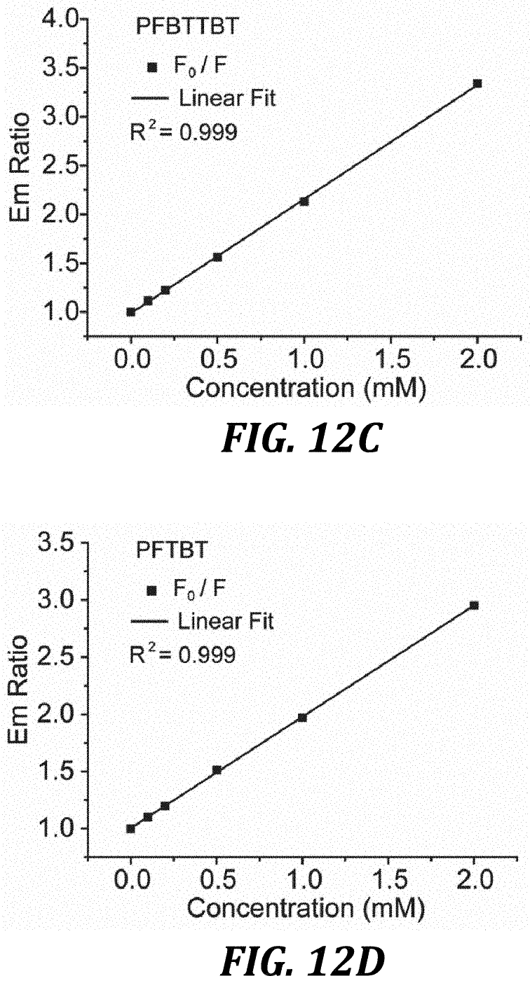

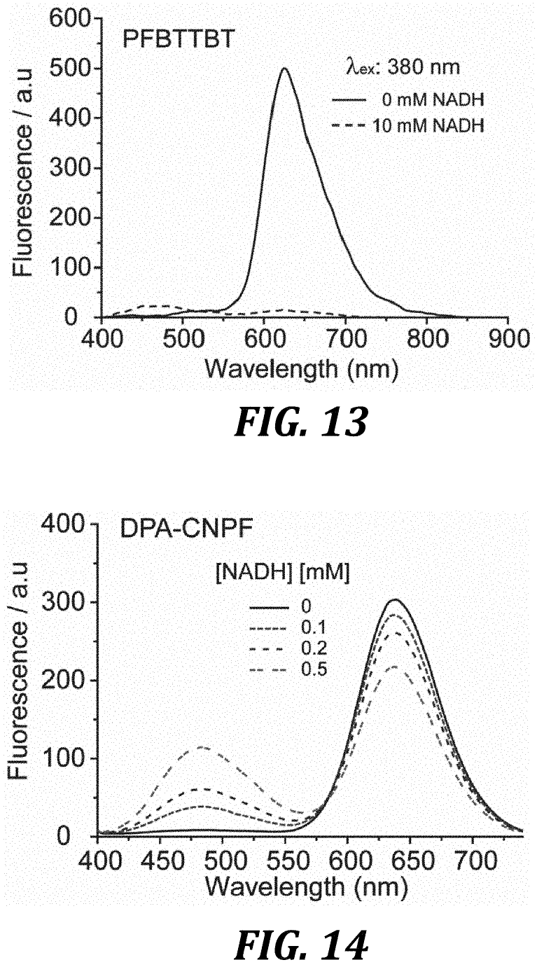

FIG. 2A shows the representative luminescence spectral changes of PFO Pdots upon addition of NADH (The assay upon NADPH showed similar spectral evolution, provided in FIG. 10). The emission is strongly quenched by increased NADH concentration. FIG. 11 depicts the Stern-Volmer plot of fluorescence intensity of PFO Pdots versus dissolved NADH. The data are fitted very well with a linear function with the NADH concentration in physiologically relevant ranges of 0-2 mM. The limit of detection (LOD) is 25 μM and quenching constant (K_{SV}) is 1.04×10³ M⁻¹. We also investigated the luminescence quenching of the other Pdots under the same conditions. PDHF Pdot has a LOD of 36 μM with a K_{SV} of 0.94×10³ M⁻¹ (FIG. 2B, FIG. 12A), PFBT Pdot has a LOD of 14 μM with a K_{SV} of 4.89×10³ M⁻¹ (FIG. 2C, FIG. 12B), PFBTTBT has a LOD of 27 μM with a K_{SV} of 1.17×10³ M⁻¹ (FIG. 2D, FIG. 12C), and PFTBT has a LOD of 28 μM with a K_{SV} of 0.97×10³ M⁻¹ (FIG. 2E, FIG. 12D). The PFBT Pdots exhibited a smallest LOD and largest K_{SV} among these Pdots, illustrating a superior sensitivity in detecting NADH. FIG. 13 graphically illustrates fluorescence emission of PFBTTBT Pdots, in accordance with an embodiment of the disclosure, before and after adding NADH (10 mM) obtained by exciting at λₑₓ=380 nm. These results demonstrate that Pdots can be promising “turn-off” fluorescent probes for NADH detection.

Ratiometric fluorescent probes rely on analyte-induced changes in emission intensity at two or more different wavelengths that greatly increase signal-to-noise ratio and improve quantification. Luminescence response of DPA-CNPPV Pdots toward NADH was investigated, the emission band changed dramatically with a large decrease in red emission at 627 nm and a concomitant increase in blue emission at 458 nm, resulting in ratiometric sensor to NADH.

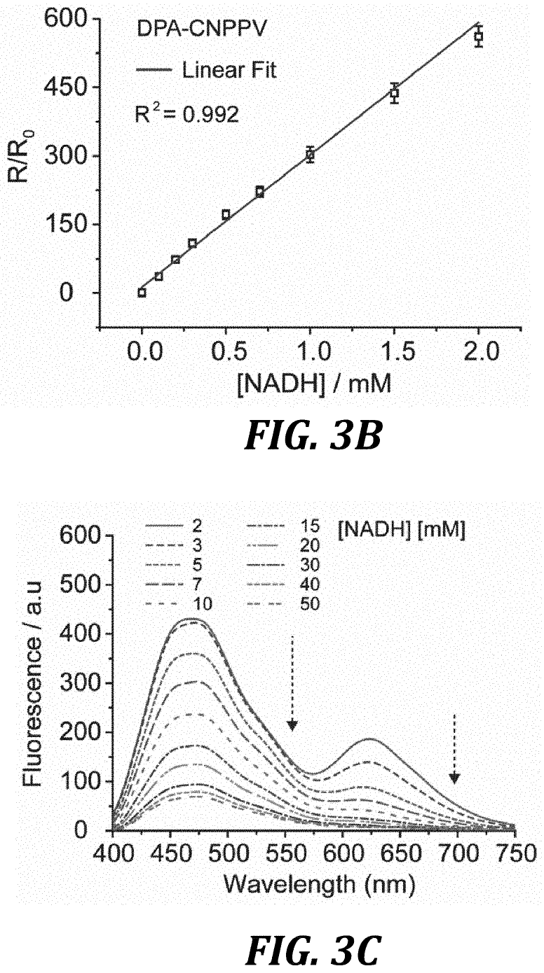

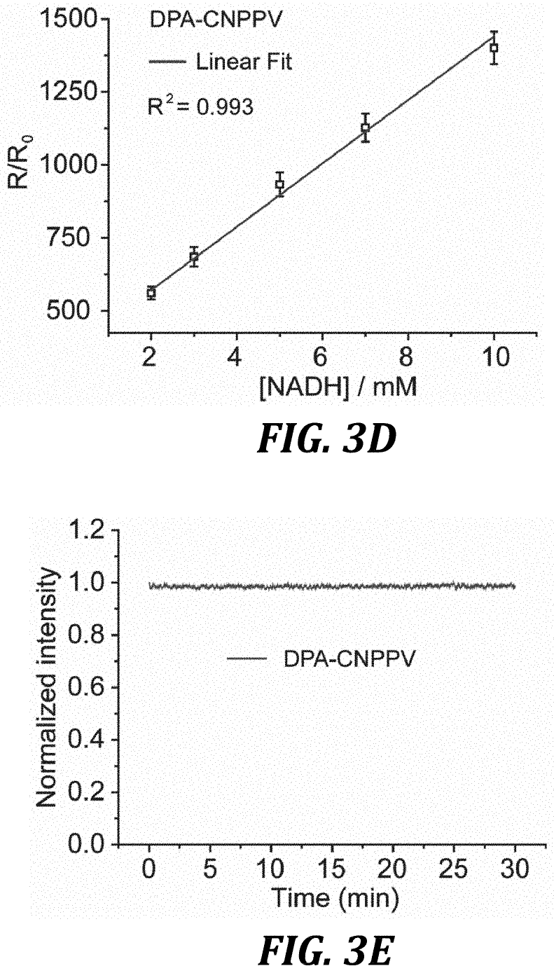

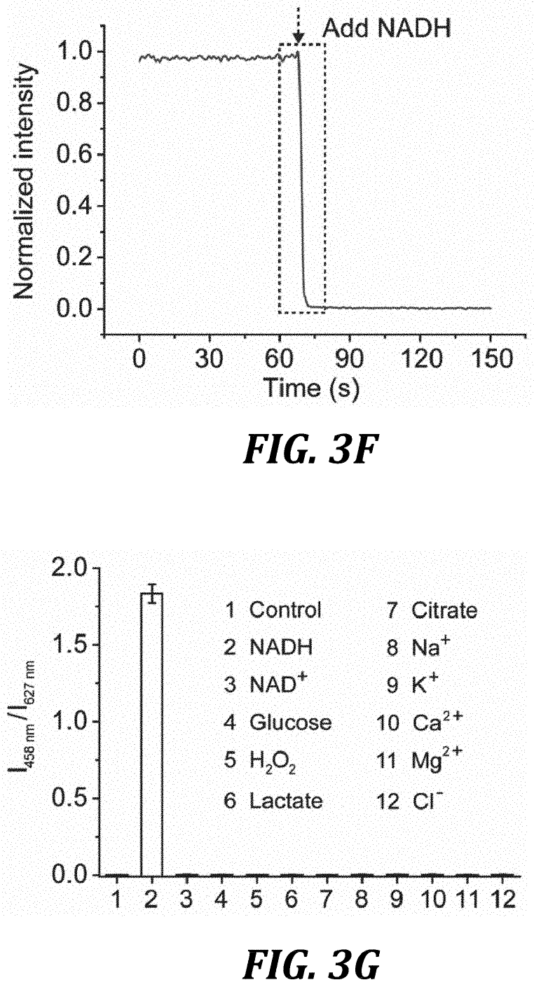

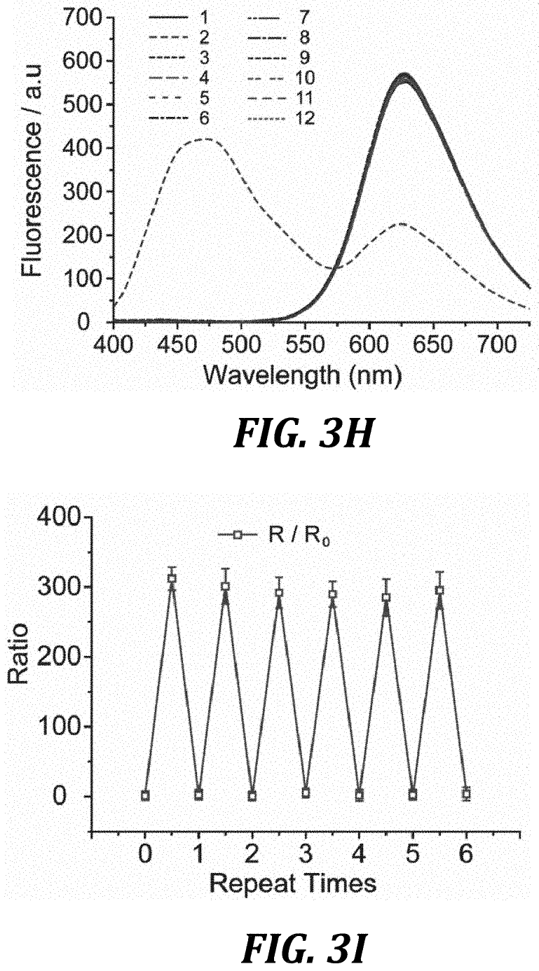

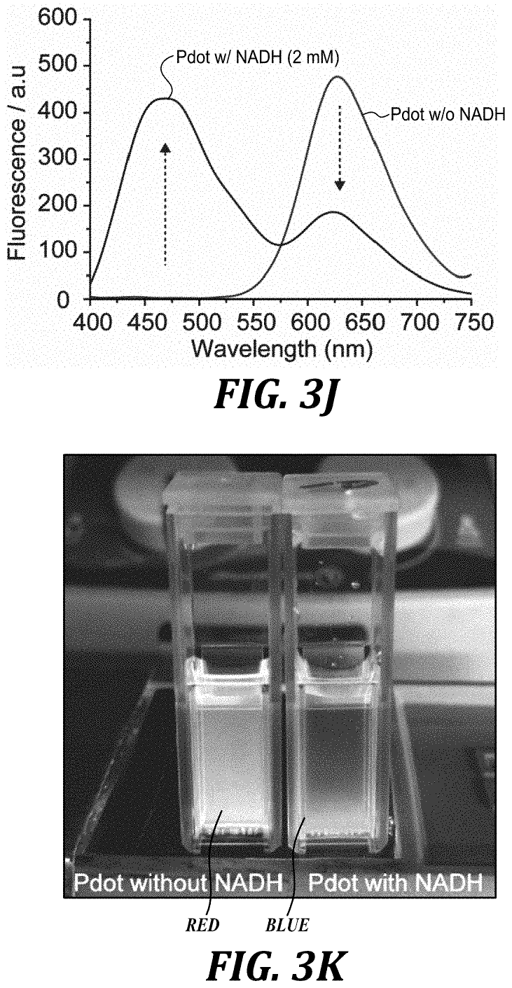

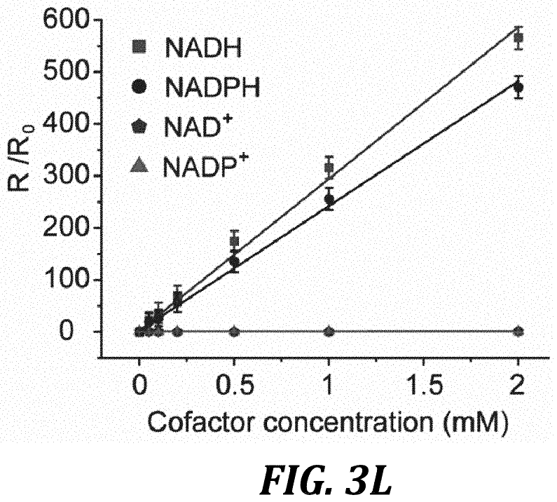

The fluorescence quantum yield (QY) of DPA-CNPPV Pdots recorded at the region of 500-800 nm decreased from 10.8% to 3.4% in the presence of 100 mM NADH, while the corresponding QY recorded at the region of 400-500 nm increased from 0.2% to 1.3% (TABLE 4). The fluorescence intensity ratio (R=I_{458 nm}/I_{627 nm}, relative variation: R/R₀; where R₀ indicates the fluorescence intensity ratio of pure Pdots in the absence of NADH and R is the fluorescence intensity ratio with different NADH concentrations) changes showed an excellent linear relationship with NADH concentrations in the wide ranges of 0-2 mM (FIGS. 3A, 3B) and 2-10 mM (FIGS. 3C, 3D), respectively. The LOD of DPA-CNPPV Pdots for NADH was determined to be as low as 3.1 μM.

TABLE 4
QUANTUM YIELD (QY) CHARACTERIZATIONS OF
DPA-CNPPV PDOTS (2 μG ML⁻¹)

| Pdots + | | | | | | | |
|---|---|---|---|---|---|---|---|
| [NADH] | 0 μM | 10 μM | 20 μM | 30 μM | 40 μM | 60 μM | 100 μM |
| QY | 10.8 | 7.6 | 6.2 | 5.4 | 4.7 | 4.0 | 3.4 |
| (500-800 nm) | | | | | | | |
| QY | 0.2 | 0.7 | 0.9 | 1.0 | 1.1 | 1.2 | 1.3 |
| (400-500 nm) | | | | | | | |

Photostability of fluorescent probes is a critical issue for long-term monitoring of analyte. It can be seen from FIG. 3E that the fluorescence intensity of DPA-CNPPV Pdots remains almost constant under the continuous illumination of 385 nm light for 30 min. Additionally, the results of the reaction kinetics demonstrated that the reaction of DPA-CNPPV Pdots with NADH cause a radical time-dependent fluorescence intensity ratio change, which is completed within 5 s (FIG. 3F), suggesting a fast response between the DPA-CNPPV Pdots probe and NADH. Selectivity is another important sensor parameter to be concerned for biosensing. The DPA-CNPPV Pdots sensor shows high selectivity in the presence of various potentially interfering substrates, including active oxidizing and reducing species, different carbohydrate derivatives, and abundant cellular cations. NADH triggers a remarkable enhancement of emission ratio of I₄₅₈ nm/I₆₂₇ nm (FIG. 3G), while spectral changes by other potentially interfering substrates up to 1 mM are not discernible (FIG. 3H). Reversibility of the ratiometric Pdots sensor is also investigated. An DPA-CNPPV Pdots sensor was repeatedly separated from NADH (1 mM) by ultrafiltration and gel filtration. The sensor's response remained unchanged for each cycle measurement (FIG. 3I), demonstrating the good reversibility of DPA-CNPPV Pdots sensor, which is also in agreement with the electrons transfer mechanism without chemical reaction. FIGS. 3J and 3K show that NADH quenches Pdot emission at 627 nm and fluoresces at 458 nm. FIG. 3J shows emission spectra of DPA-CNPPV Pdots and NADH at 0 and 2 mM NADH with excitation at 385 nm. FIG. 3K shows photograph of Pdot solutions illuminated with UV light at 365 nm. FIG. 3L shows fluorescence response of the sensor to NADH and NADPH. FIG. 3L shows fluorescence response of the sensor to NADH and NADPH. Titration of the DPA-CNPPV/PSMA Pdot with NADH, NADPH, NAD⁺, and NADP⁺ showed a fluorescence response only to NADH and NADPH, indicating that NAD⁺ and NADP⁺ do not quench the Pdot emission, and do not themselves emit at 458 nm under UV illumination.

We also investigated the luminescence performance of other DPA-based Pdots (DPA-CNPF) with NADH, which yielded a similar result as of DPA-CNPPV (FIG. 14). Fast, sensitive, selective, and reversible response to NADH make Pdot sensor have a great potential for monitoring metabolites.

### Example 5: Detecting Nadh in Living Cells

After confirming the sensitivity, selectivity, and stability of the Pdots probe, we next explored the potential application of Pdots for imaging NADH in live cells. The present Example demonstrates pdots in a common solvent with NADH.

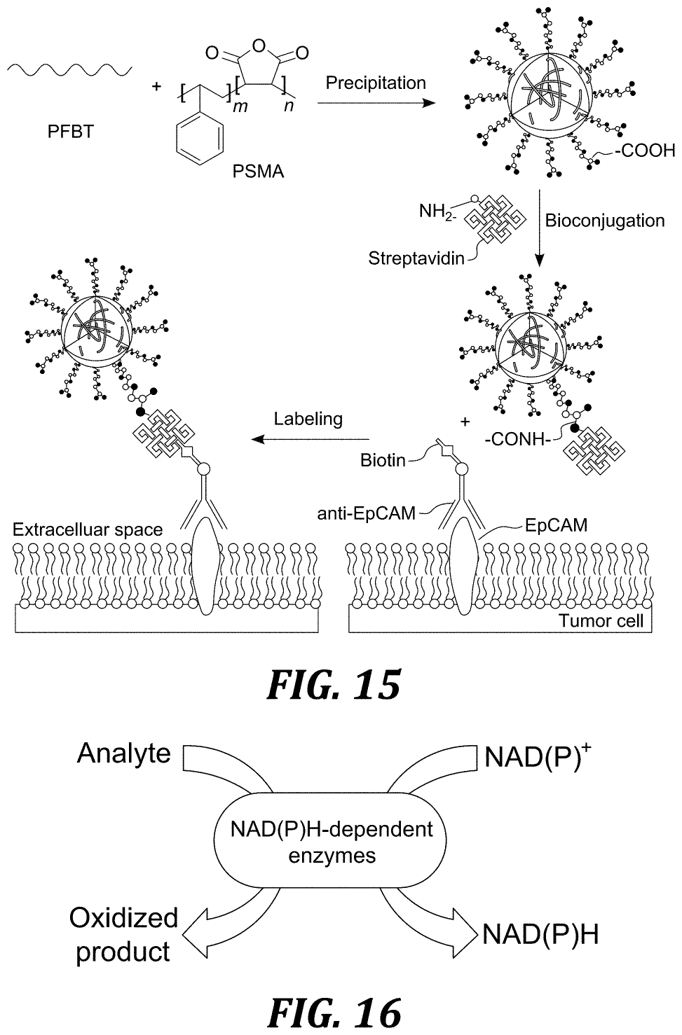

Pdots with ultrabright fluorescence have been successfully applied for specific cell labeling. Here, we choose PFBT Pdots as an example for in vitro application. The streptavidin functionalized Pdots are used to label a specific cellular target, protein EpCAM, for the detection of circulating tumor cells MCF-7 (FIG. 15).

Pdots-labeled MCF-7 cells were incubated with PBS and NADH, respectively. Breast cancer cell line MCF-7 cells were purchased from American Type Culture Collection (Manassas, Va., USA). Primary cultured MCF-7 cells were cultured at 37° C. in Dulbecco's Modified Eagle Medium (DMEM) containing 10% fetal bovine serum and 1% penicillin/streptomycin with a humidified environment containing 5% CO₂. The culture medium was changed every two days. Cells were detached at 80% confluence using 0.25% trypsin-EDTA, then centrifuged at 800 rpm for 5 min. Pellets were resuspended in culture medium and sub-cultured in culture flasks.

We performed bioconjugation by using an EDC-catalyzed reaction between carboxyl groups on the Pdots and amine groups on the biomolecule streptavidin. In a typical bioconjugation reaction, 80 μL of polyethylene glycol (PEG, 5% w/v) and an equal amount of concentrated HEPES buffer (1 M) were added to 4 mL of a PFBT Pdot solution (50 μg/mL, in Milli-Q water), resulting in a Pdot solution in 20 mM HEPES buffer with a pH of 7.3. Then, 240 μL of streptavidin (5 mg/mL) was added to the solution and vortexed. Next, 80 μL of a freshly prepared EDC solution (5 mg/mL, in deionized water) was added and the resulting mixture was left on a rotary shaker for 4 h at room temperature. The resulting Pdot-streptavidin bioconjugates were separated from free biomolecules by gel filtration using Sephacryl HR-300 gel media. For labeling the surface marker EpCAM, MCF-7 cells were harvested from the culture flask, washed, centrifuged, and resuspended in a labeling buffer (1×PBS, 1% BSA). The MCF-7 cells were dispersed in 100 μL of labeling buffer in a 5 mL round-bottom tube, and sequentially incubated with a biotinylated primary antiEpCAM (0.5 mg/mL) and Pdot-streptavidin. The Pdot-tagged MCF-7 cells were then incubated at 37° C. for 30 min in PBS solution (10 mM, pH=7.4) in the absence or presence of NADH (1 mM). Fluorescence imaging was performed on a fluorescence microscope with a 20× objective. The excitation light was provided by a xenon lamp and filtered by a band-pass filter (Semrock FF01-350/52). The fluorescence signal was filtered by a band-pass filter (Semrock FF01-525/20). Images processing and analysis was carried out on Image J and Matlab software.

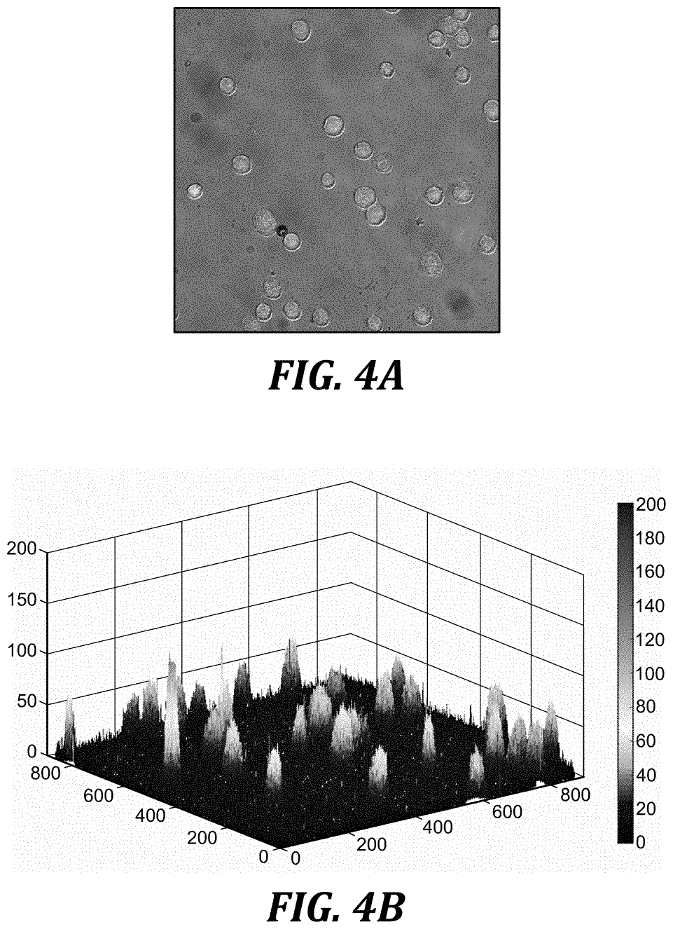

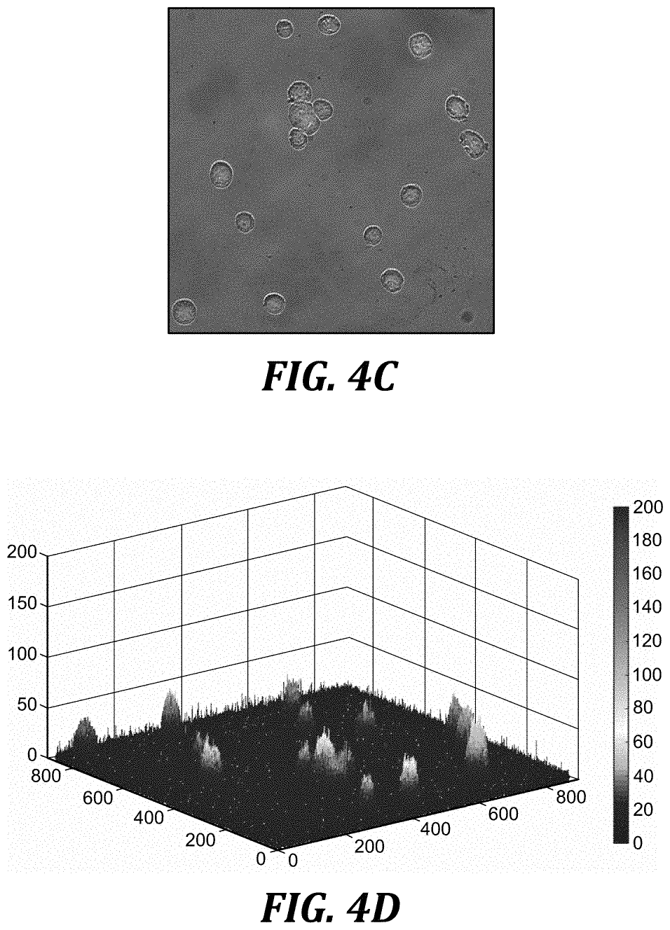

FIG. 4A shows merged bright-field and fluorescence images of PFBT Pdots-labeled MCF-7 cells in PBS without NADH. Pdots-labeled MCF-7 cells exhibit the strong fluorescence. As compared with the control group, the cellular fluorescence which receive NADH is apparently decreased (FIG. 4C). The above differentiation in fluorescence intensity is presented more vividly by their 3D interactive intensity (FIGS. 4B and 4D). Fluorescence intensity of Pdots is inversely proportional to NADH concentration, indicating successful detection of NADH in live cells by the Pdot sensor.

### Example 6: Ratiometric NAD(P)H Sensing Using Digital Cameras or Smartphones for Point-of-Care (POC) Testing and In Vivo Imaging

The present Example demonstrates pdots dispersed in a common solvent with NADH.

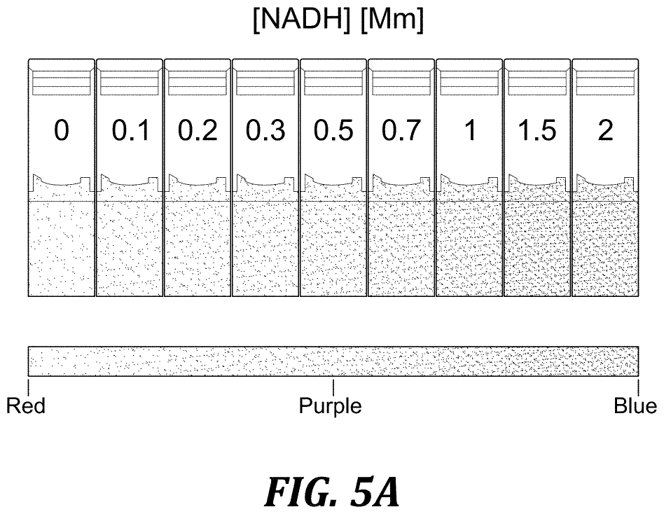

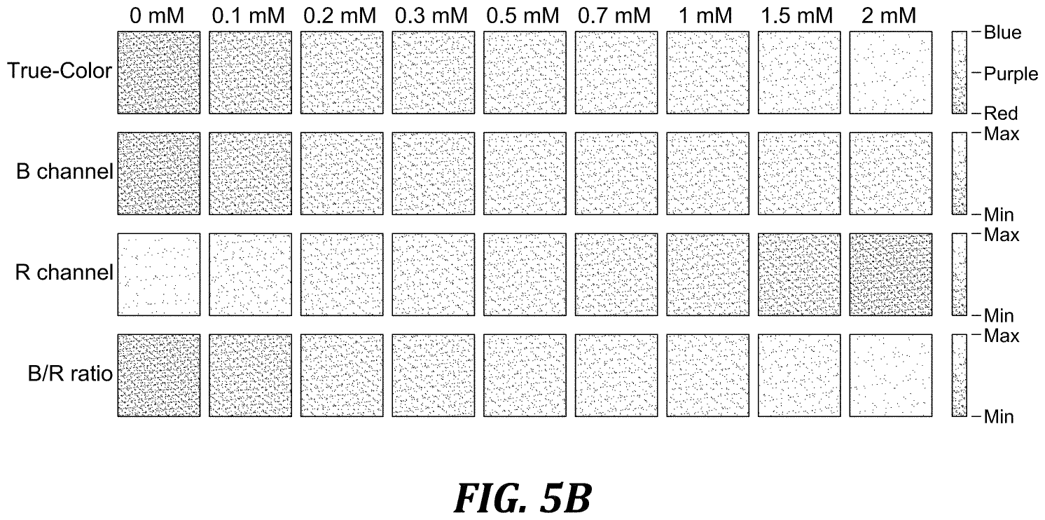

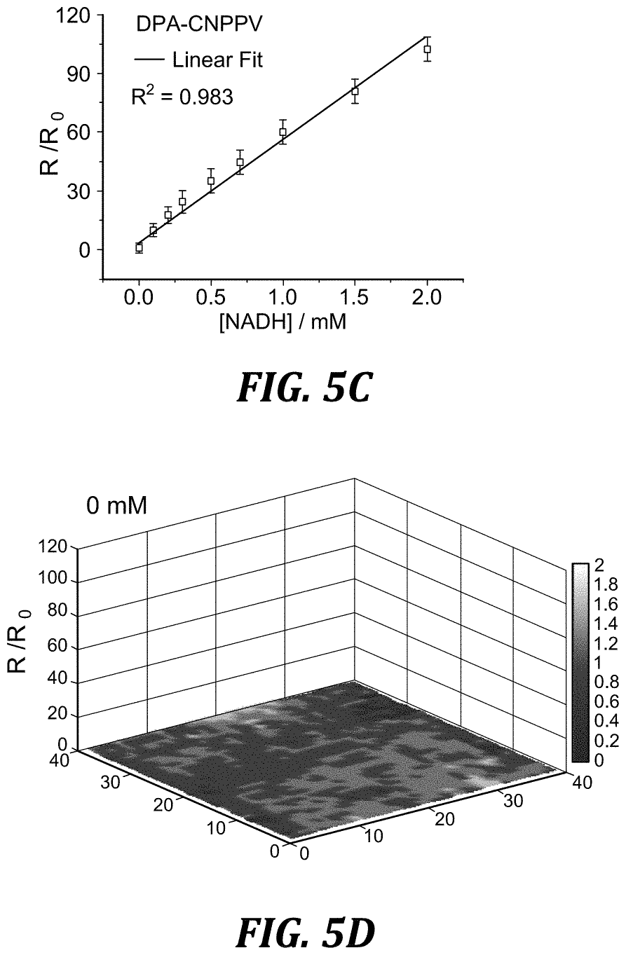

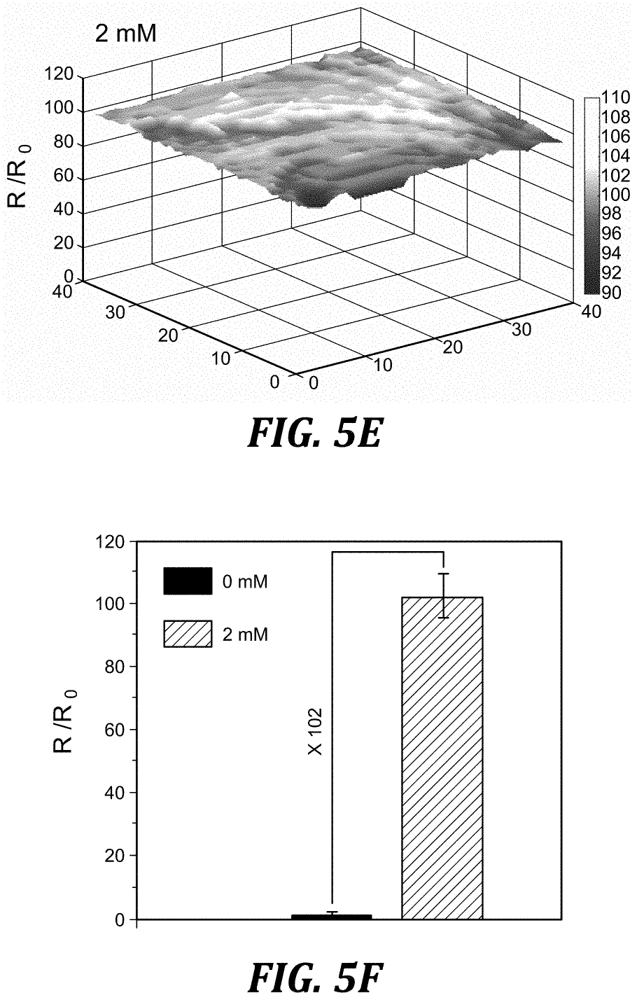

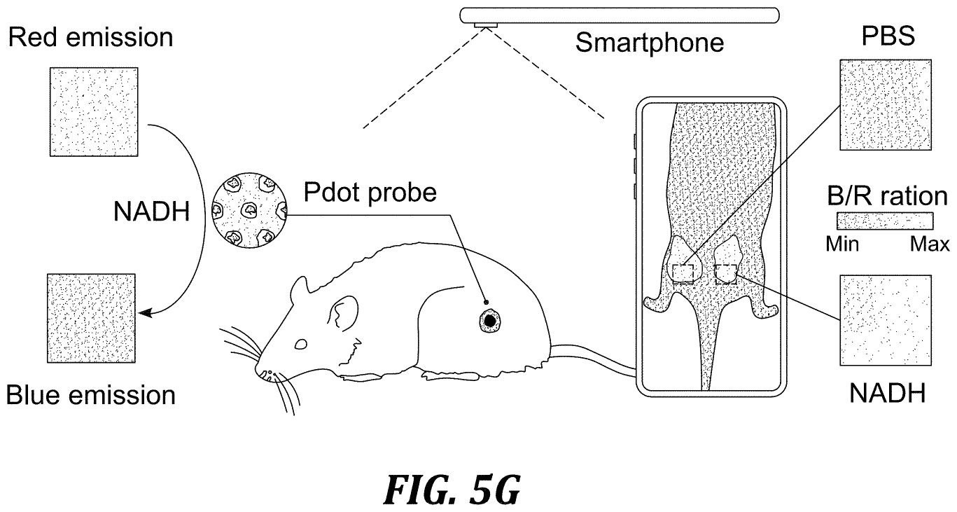

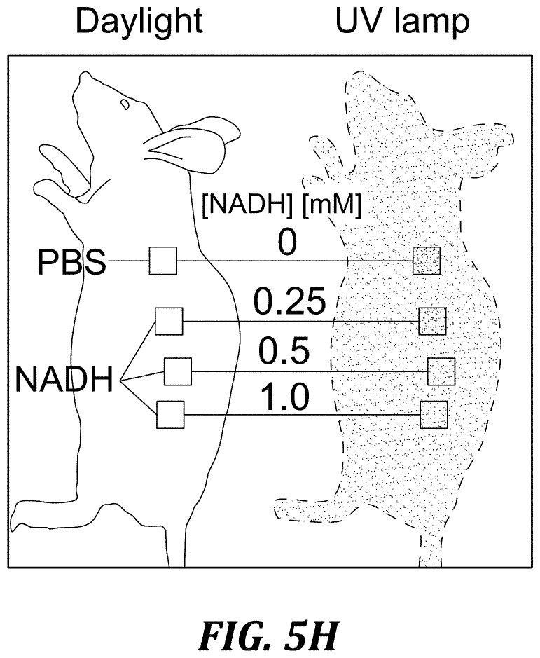

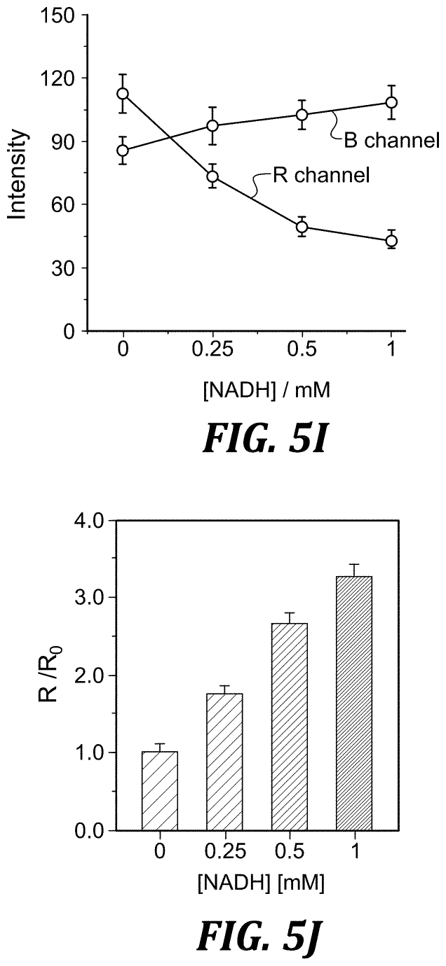

Recent years have witnessed major innovations in biosensors for POC applications, which allowing individuals to perform simple diagnostic or prognostic tests without visiting a medical laboratory or hospital, thereby improving the convenience. This also increases the likelihood that doctor will receive the results quicker, which allows for better immediate clinical management decisions to be made. Digital and smartphone camera-based POC testing is rapidly emerging as a potential technology to generate moveable diagnostic and monitoring systems for POC testing owing to economic considerations and availability of equipment. As shown in FIG. 5A, a red-to-blue solution fluorescent color change could be directly visualized by the smartphone camera for DPA-CN-PPV Pdots after incubation with NADH at physiologically relevant range. In the absence of NADH, the solution fluorescent color was red, with the increase in NADH concentrations, the emission color finally turned to blue. The large ratiometric variation also allowed the clear visualization of the solution's fluorescent color change. Each of the primary true-color images can be split into red (R), green (G), blue (B) channels. After digitized by using an image-processing algorithm (FIG. 5B), the intensity ratio of B/R channels is used for quantification of the NADH concentrations. FIG. 5C displayed the linear response of intensity ratio (R=B/R) in the physiological relevant ranges of 0-2 mM. The maximum ratio enhancement could reach more than 100-fold when NADH concentration increased from 0 to 2 mM (FIG. 5D-5F). These results indicated that the ratiometric DPA-CNPPV Pdots sensor combined with the smartphone camera or a digital camera provides a viable approach for NADH monitoring.

In addition, the feasibility of the DPA-CNPPV Pdots for the in vivo imaging of NADH is next evaluated by processing the fluorescence of Pdots imaged by the smartphone under a UV-lamp excitation (FIG. 5G-5J).

Here, female Balb/c nude mice were used in compliance with the Guidelines for the Care and Use of Research Animals. Nude mouse under anesthesia was given a subcutaneous injection of DPA-CNPPV Pdots in PBS together with different concentrations of NADH (at 0 mM, i.e. no NADH, and at 0.25, 0.5, and 1.0 mM NADH) into the dorsal area of nude mice. UV lamp was used for illumination and a smartphone camera for imaging (FIG. 5G). FIG. 5H shows the concentration-dependent sensing at the various NADH concentrations (0.25, 0.5, and 1.0 mM). True-color images of regions of interest were split into blue- and red-channel images to calculate the B/R ratio (FIGS. 5I, 5J). The B/R ratio showed excellent linearity in the Pdot sensor's in vivo NADH detection response.

### Example 7: Pdot Sensor for PKU

The present Example demonstrates an NADH-dependent enzyme coupled to a pdot and an NADH-dependent enzyme coupled to a common substrate with a pdot.

Metabolites play highly important roles in all aspects of living organisms because they have various functions such as energy conversion, structure, signaling, epigenetic influence, cofactor activity, and interactions with other organisms. NAD⁺/NADH and NADP⁺/NADPH is a cofactor that is central to metabolism. The total number of NAD⁺/NADH and NADP⁺/NADPH participating reactions is over 500. Most of these reactions involve dehydrogenases. Dehydrogenases are enzymes belonging to the group of oxidoreductases that usually oxidize substrates of interest by reducing NAD⁺. The stoichiometrically produced NADH can be quantified by the NADH-sensitive Pdots sensor. The level of NADH corresponds to the level of the substrate in the sample (FIG. 16). Here, our strategy for metabolites detection is based on the integration of an NADH-sensitive Pdots sensor with an NADH-dependent enzyme that catalyzes the analyte of interest oxidation reactions. Our strategy for metabolites detection also can be based on the integration of an NADPH-sensitive Pdots sensor with an NADPH-dependent enzyme that catalyzes the analyte of interest oxidation reactions (FIG. 16).

As the first application of our NADH-sensitive Pdots sensor, we developed a smartphone-based assay for Phenylketonuria (PKU). PKU is an inherited disorder of metabolism of the essential amino acid phenylalanine due to a defect in phenylalanine hydroxylase (PAH). Infants and children with PKU typically develop signs of gradual, progressive neurological disease. To quantify phenylalanine, we used DPA-CNPPV Pdots sensor with the enzyme phenylalanine dehydrogenase (PheDH), which has a high specific activity toward phenylalanine, for measurements (FIG. 6A). Defined concentrations of phenylalanine were spiked into Pdots sensor (contains 0.05 mg/mL DPA-CNPPV Pdots, 3 mM NAD⁺, 1 μM PheDH, 200 mM glycine buffer at pH 10.5). After 10 min incubation, the emission ratio of the sensor in the reaction mixture was measured using a fluorescence spectrometer.

The resulting luminescent sensor showed an 18.9-fold change in the emission ratio when titrated with phenylalanine. The LOD of Pdots for phenylalanine was determined to be 3.5 μM. The c₅₀ of the sensor (the phenylalanine concentration resulting in 50% of the maximum sensor response) was measured to be 279.7 μM. (FIG. 6B).

PKU patients should be managed from birth with all available options to control blood phenylalanine levels for whole life. This is important because phenylalanine rises to toxic levels in the blood causing irreversible brain damage and neurological complications. PKU patients are classified according to their blood or serum or plasma phenylalanine concentrations before treatment: newly diagnosed newborn infant with levels of 120 to 360 μM denote benign mild hyperphenylalaninaemia (HPA); levels of 360 to 600 μM, mild HPA; levels of 600 to 900 μM, mild PKU; levels of 900 to 1200 μM, moderate PKU; and levels exceed 1200 μM, classic PKU. As shown in FIG. 6C-6I, the ratio of emission showed linear relationships with the phenylalanine level in different clinical symptoms relevant range of blood phenylalanine. For healthy level, Pdots sensor has a resolution of 2.6 μM with a sensitivity of 4.89×10⁴ M⁻¹. Performance parameters of PKU sensor in other dynamic range including maximal signal change, sensitivity, and resolution are summarized in TABLE 5.

TABLE 5
SUMMARY OF PERFORMANCE PARAMETERS OF PKU SENSOR

| | | R/R₀ | Dynamic | | | | |
|---|---|---|---|---|---|---|---|
| | [Phe] | (R = I₄₅₈ | range | Δc | | | R |
| Diagnose | (μM) ^{a} | nm/I₆₂₇ nm) | (μM) ^{b} | (μM) ^{c} | Δsₘₐₓ ^{d} | S (M⁻¹) ^{e} | (μM) ^{f} |
| Health level | ~60 | <5.9 | 0-120 | 120 | 488% | 4.079 × 10⁴ | 2.6 |
| Benign mild | 120-360 | 5.9-11.5 | 120-360 | 240 | 96.5% | 2.399 × 10⁴ | 5.4 |
| HPA | | | | | | | |
| Mild HPA | 360-600 | 11.5-14.7 | 360-600 | 240 | 27.2% | 1.324 × 10⁴ | 11.2 |
| Mild PKU | 600-900 | 14.7-16.8 | 600-900 | 300 | 14.1% | 6.84 × 10³ | 25.4 |
| Moderate | 900-1200 | 16.8-18.0 | 900-1200 | 600 | 7.3% | 4.15 × 10³ | 49.3 |
| PKU | | | | | | | |
| Classic PKU | 1200-2400 | >18.0 | 1200-1800 | 600 | 6.5% | 1.93 × 10³ | 110.9 |
| | | >18.0 | 1200-2400 | 600 | 3.9% | 1.29 × 10³ | 172.0 |

^{a} Concentration of phenylalanine in plasma.
^{b} Linear range of biosensor.
^{c} Maximal signal change over the dynamic range.
^{d} Sensitivity (S = Δs/Δc; .s = R/R₀, c = [Phe]).
^{e} Resolution(R = σ/S).

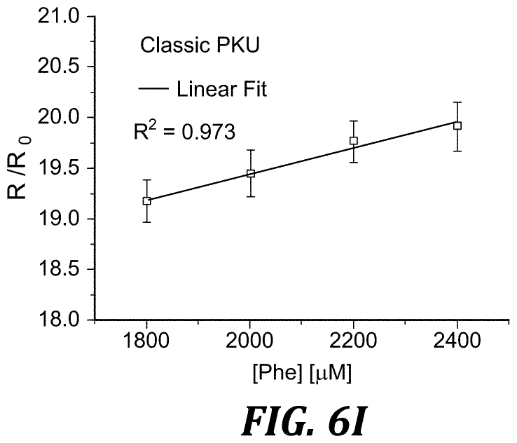

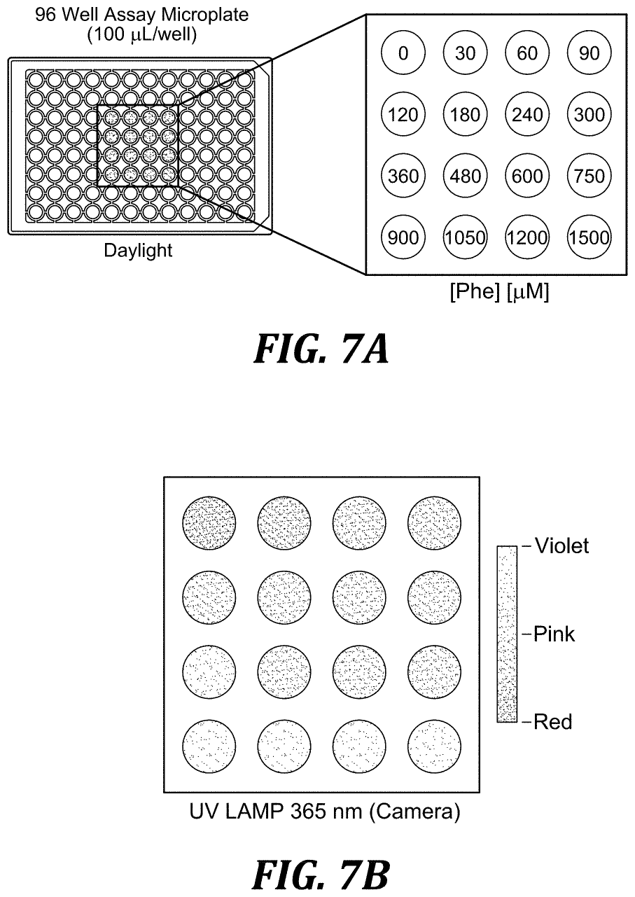

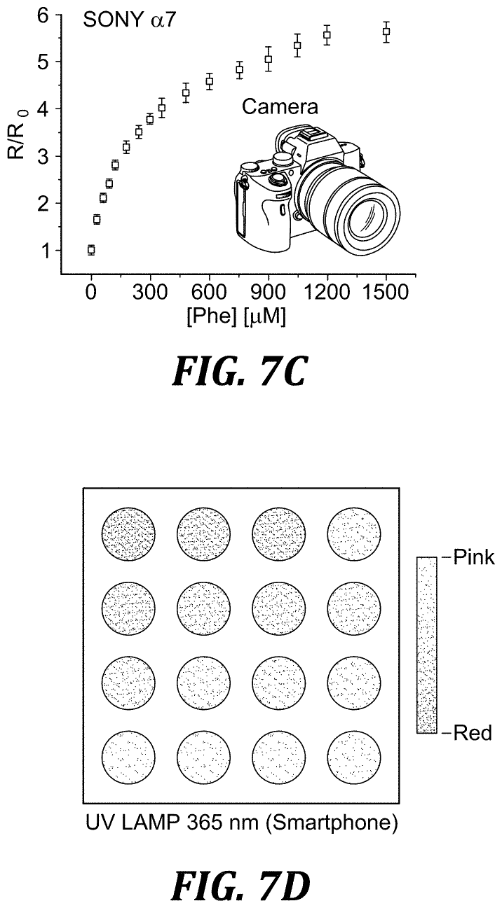

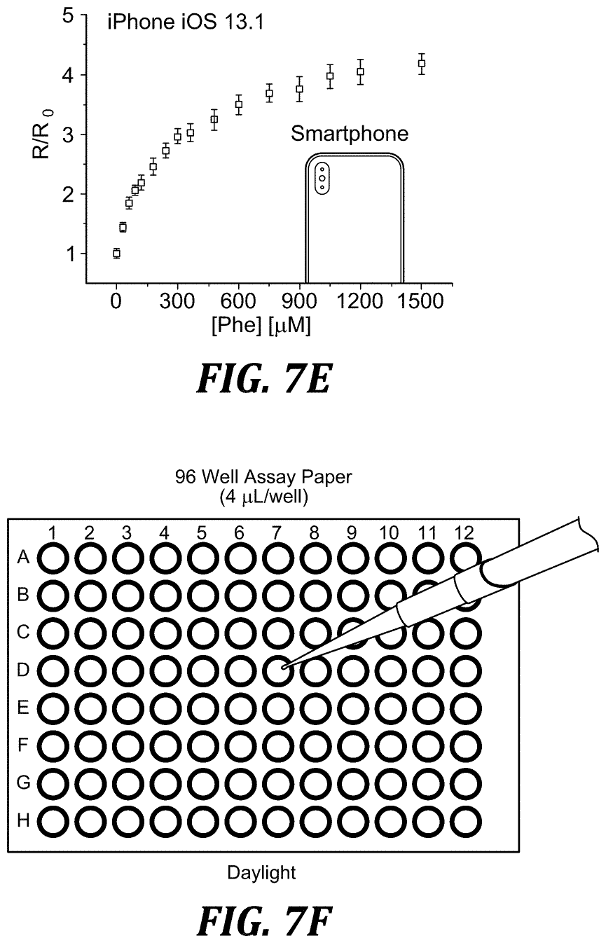

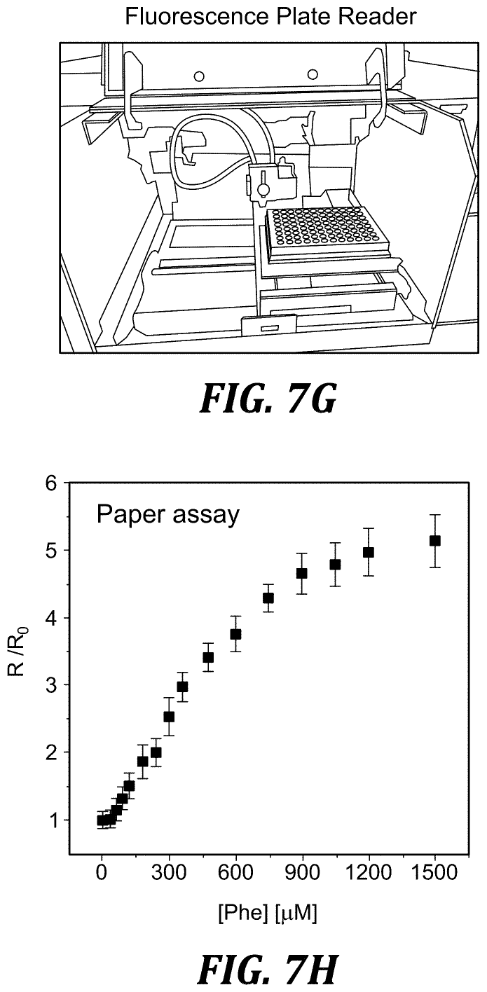

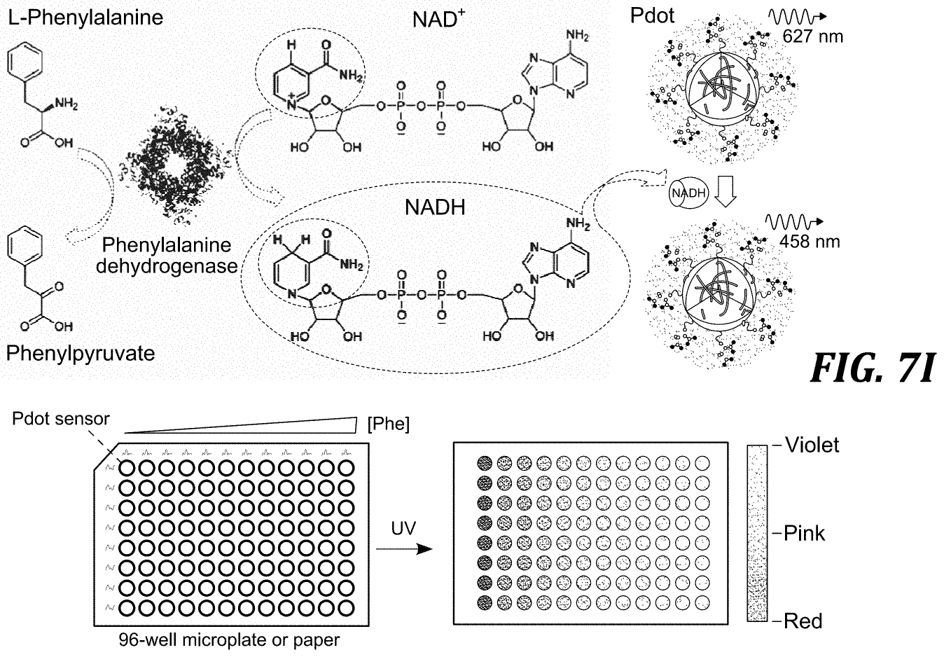

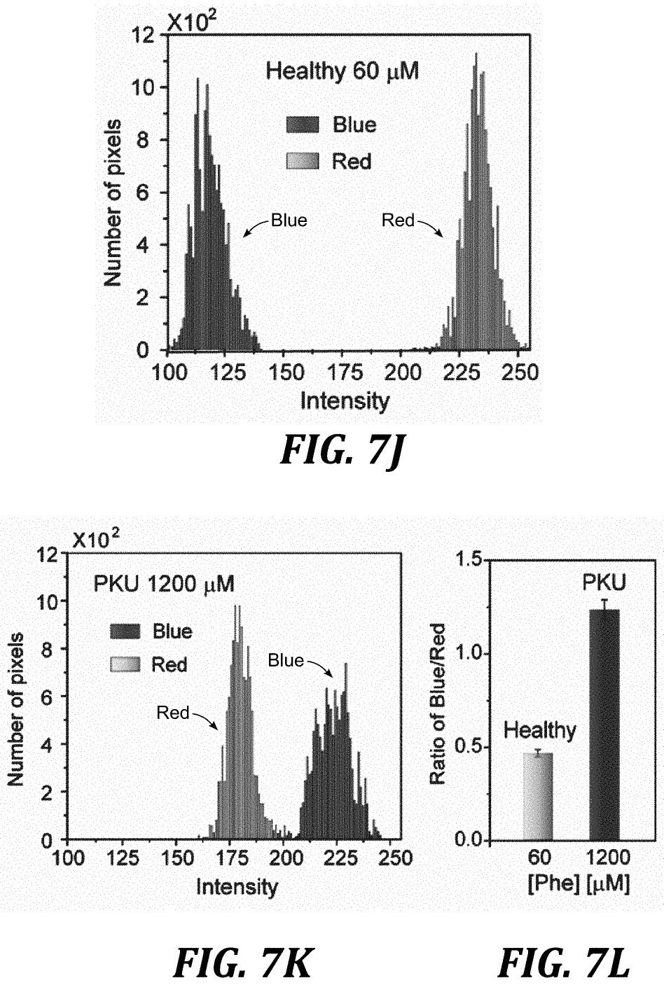

Frequent blood tests measure the level of phenylalanine in a child's blood to work towards preventing health problems. Numerous methods and regents, including Guthrie bacteria inhibition assay method, ferric chloride test, dye-based fluorescence assay, and PCR method have been developed for phenylalanine quantification. However, these methods typically involve complex preparation or long test time. Therefore, development of new materials and methods that can detect phenylalanine at home in a facile, rapid, and accurate manner is of considerable significance. To perform self-testing phenylalanine measurements, we designed an assay by using 96 well microplate and the digital camera (FIG. 7A). This enzymatic assay requires a 10 min incubation. Then phenylalanine concentrations can be calculated from the emission ratios (R/R₀) of ROI images (FIG. 7B). The photographs taken by the digital camera and smartphone appear different due to a different color temperature setting (FIG. 7D, 7E). For 96 well assay microplate, A SONY a7 camera (file format: RAW; WB: 3000, ISO: 2000; Shutter speed: 1/20) was used to capture photos. A smart-phone camera (iPhone iOS 13.1, auto mode) was used as an alternative to capture photos. The ratio was used to calculate the phenylalanine concentration. Furthermore, we have demonstrated its promising potential for paper-based sensors.

FIG. 7I is an example illustration of the scheme. Here we designed a metabolite biosensor that combines an NAD(P)H-sensitive Pdot and a metabolite-specific NAD(P)H-dependent enzyme in a solution- or paper-based assay (FIG. 7I). Enzyme-catalyzed oxidation of the metabolite generates NAD(P)H; under ultraviolet (UV) illumination, the NAD(P)H quenches the red emission of the Pdot while also fluorescing in the blue region. The NAD(P)H-sensitive Pdot consists of the luminescent conjugated polymer poly[{2-methoxy-5-(2-ethylhexyloxy)-1,4-(1-cyanovinylenephenylene)}-co-{2,5-bis(N,N′-diphenylamino)-1,4-phenylene}] (DPA-CNPPV) and the amphiphilic polymer poly(styrene-co-maleic anhydride) (PSMA). Excitation of the Pdot with UV illumination results in emission at 627 nm that is quenched by NAD(P)H, along with emission by NAD(P)H at 458 nm. The ratio of emission intensities at 458 nm and 627 nm—or the ratio of blue-to-red-channel emission intensities when using a digital camera or cell phone and RGB image processing—is used to accurately measure the concentration of the oxidized metabolite.

Grade 1 Chr Cellulose Chromatography Paper (GE Healthcare) were patterned with 96-well plate template using a HP Pro400 M401 dne printer. Paper-based assays were prepared by lyophilizing 4 μL of buffer containing 0.05 mg/mL DPA-CNPPV Pdots, 3 mM NAD⁺, 2 μM PheDH, 200 mM glycine at pH 10.5 onto paper disks. A cold bath of liquid nitrogen was used during the first 2 hours of lyophilization. For measurement, 4 μL of analyte (in buffer at pH 10.5) was added onto the test paper directly. After 10 min incubation, the emission ratio of the sensor on the test paper was measured using a fluorescence plate reader or digital camera or a cell-phone camera. Blank paper was used as a control to eliminate background fluorescence interference from the paper.

We lyophilized dehydrogenase and the NADH-sensitive Pdots onto 96 well test paper. Only 0.4 μL of analyte is needed to add to the test paper containing the buffer and lyophilized sensor (FIG. 7F), then the results can be analyzed by using a fluorescence plate reader (FIG. 7G) or digital camera or cell phone (FIG. 7C and (FIG. 7E). The enzymatic reaction was initiated by adding a small volume of analyte-containing sample onto the test paper (FIG. 7F). Images were analyzed with an RGB image-processing algorithm to calculate the average blue- and red-channel intensities within each well from pixel intensity distributions (FIG. 7J to 7L). The ratio of blue- and red-channel intensities was significantly lower at 60 μM Phe (healthy) than at 1200 μM Phe (classic PKU threshold) (FIG. 7L). As shown in FIG. 7H, the phenylalanine measurement by the paper-based system clearly reflected the PKU levels. The simple and rapid assay procedure make it possible for patients to get a point-of-care self-testing. TABLE 6 illustrates the performance of the Pdot sensor for paper assay using a digital camera (e.g. from cell phone or a standalone camera) for readout.

TABLE 6
Phenylalanine Pdot biosensor performance for paper
assay using a digital camera for readout.

| | | s (solution assay, | s (paper assay, |
|---|---|---|---|
| Diagnosis | [Phe] (μM) ^{a} | camera) ^{b} | camera) |
| Healthy | ~60 | <2.8 | <1.5 |
| Benign mild HPA | 120-360 | 2.8-3.9 | 1.5-2.9 |
| Mild HPA | 360-600 | 3.9-4.6 | 2.9-3.8 |
| Mild PKU | 600-900 | 4.6-5.1 | 3.8-4.6 |
| Moderate PKU | 900-1200 | 5.1-5.6 | 4.6-5.0 |
| Classic PKU | 1200-2400 | >5.6 | >5.0 |

^{a} Concentrations of phenylalanine found in plasma.
^{b} s = R/R₀ (blue-to-red-channel emission ratio).

### Example 8: Assaying Human Blood Plasma Samples Using Pdot Sensor for PKU

The present Example demonstrates an NADH-dependent enzyme coupled to a common substrate as a pdot.

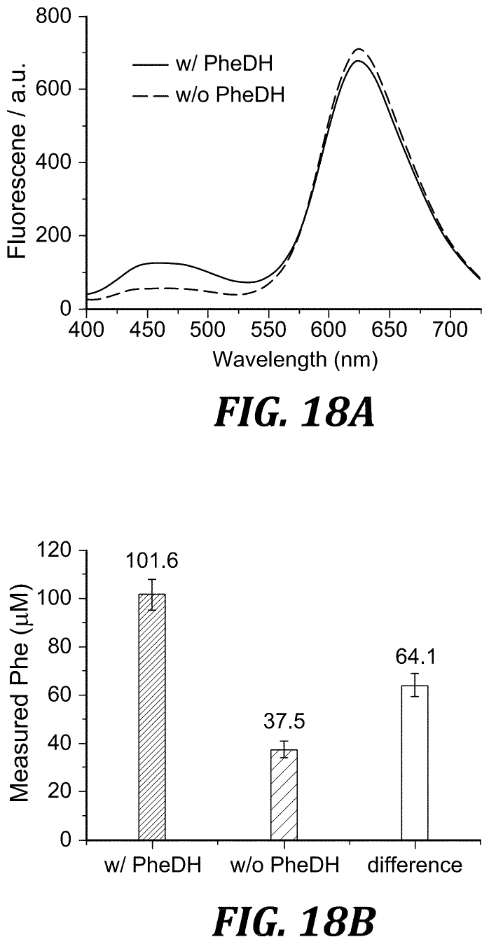

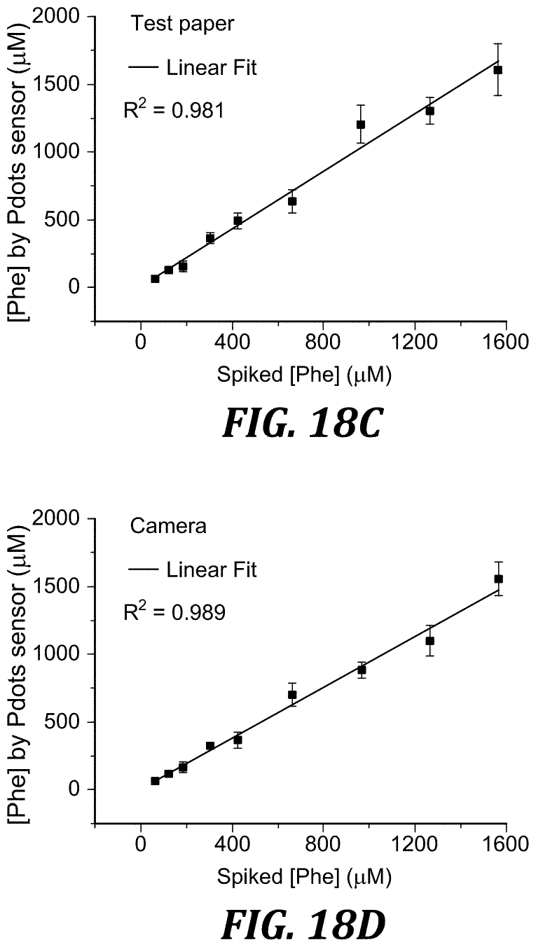

In this regard, we next evaluated the performance of the paper-based assay when assaying human blood plasma samples. The biosensor was calibrated by analyzing samples in the absence or presence of PheDH (FIG. 18A) to correct for interpatient variations in endogenous NADH concentration in blood. We subtracted the value obtained without PheDH from the value obtained with PheDH to obtain the phenylalanine concentration (labeled “difference” in FIG. 18B). As a proof of principle for PKU screening applications, we analyzed plasma samples spiked with various concentrations of phenylalanine with the paper assay, and compared the results obtained when using a plate reader (FIG. 18C) and a digital camera (FIG. 18D) for readout.

To correct for endogenous NADH in blood, whole blood (with EDTA) from healthy human donors was obtained from PlasmaLab International (Everett, USA). Plasma was separated from whole blood by centrifugation. Correction of endogenous NADH was achieved by using a Pdot sensor with and without PheDH enzyme. Plasma (0.2 mL) was added into a 0.8 mL solution containing 0.0625 mg/mL DPA-CNPPV/PSMA Pdot, 3.75 mM NAD⁺, 0 or 2.5 μM PheDH, and 250 mM glycine buffer at pH 10.5, and the mixture was incubated for 10 min. The reaction without PheDH measures endogenous NADH, and the reaction with PheDH measures endogenous NADH plus NADH produced by conversion of phenylalanine; subtracting the two values yields the phenylalanine concentration.

To prepare paper-based assay for measurement of phenylalanine in blood plasma, Grade 1 Chr cellulose chromatography paper was patterned as described above. Test paper was prepared by lyophilizing 4 μL of buffer containing 0.05 mg/mL DPA-CNPPV/PSMA Pdots, 3 mM NAD⁺, 2 μM PheDH, and 200 mM glycine at pH 10.5 onto paper disks. A cold bath of liquid nitrogen was used during the first 2 h of lyophilization.

### Example 9: Pdot Sensor for Other Metabolic Diseases, Drug Metabolisms, or Metabolites

The present Example demonstrates pdots dispersed in a common solvent with NADH-dependent enzymes.

Any metabolite that can be oxidized with NAD⁺ or NADP⁺ can be analyzed or measured with our sensor described here. As non-limiting examples, we accurately analyzed samples with various analyte concentrations using NADH-dependent enzymatic reactions for lactate, glutamate, glucose, and β-hydroxybutyrate (BHB) (FIG. 17A-17H). Here, different amounts of lactate, glucose, glutamate, or β-hydroxybutyrate analyte were spiked into the corresponding Pdot sensor solution. For lactate, the solution comprised of 0.05 mg/mL Pdot, 3 mM NAD⁺, 1 μM lactic dehydrogenase, 200 mM glycine buffer, pH 9.8; for glucose: 0.05 mg/mL Pdot, 3 mM NAD⁺, 1 μM glucose dehydrogenase, 50 mM HEPES buffer, pH 8.0; for glutamate: 0.05 mg/mL Pdot, 3 mM NAD⁺, 1 μM glutamic dehydrogenase, 50 mM HEPES buffer, pH 7.3; for β-hydroxybutyrate: 0.05 mg/mL Pdot, 3 mM NAD⁺, 1 μM β-hydroxybutyrate dehydrogenase, 50 mM HEPES buffer, pH 7.8. Lactate detection is important in medical conditions including hemorrhage, respiratory failure, hepatic disease, and sepsis; glucose monitoring is important in managing diabetes; glutamate monitoring is useful in diagnosis and monitoring of neurodegenerative diseases; and β-hydroxybutyrate sensing is used to detect hyperketonemia. Over 500 NAD(P)H-dependent enzymes and over 300 related metabolites are compatible with this system (see, e.g., TABLE 2), including over 100 medically-relevant metabolites (see, e.g. TABLES 1 and 2).

While illustrative embodiments have been illustrated and described, it will be appreciated that various changes can be made therein without departing from the spirit and scope of the invention.

1. A nanoparticle transducer for analyte concentration measurements, the nanoparticle transducer comprising:
a nanoparticle comprising a chromophore; and
a nicotinamide adenine dinucleotide (NADH)-dependent enzyme or nicotinamide adenine dinucleotide phosphate (NADPH)-dependent enzyme physically associated with the nanoparticle and configured to catalyze a reaction comprising a plurality of reaction elements;
wherein the plurality of reaction elements comprises one or more reactants including the analyte and one or more products, and wherein an amount of fluorescence emitted from the chromophore is determined by a concentration of a reaction element of the plurality of reaction elements.

2-14. (canceled)

15. A transducer substrate for analyte concentration measurements, the transducer substrate comprising:
a nanoparticle comprising a chromophore coupled to a substrate; and
an enzyme coupled to the substrate and configured to catalyze a reaction comprising a plurality of reaction elements,
wherein the plurality of reaction elements comprises one or more reactants including the analyte and one or more products, and wherein an amount of fluorescence emitted from the chromophore is determined by a concentration of a reaction element of the plurality of reaction elements.

16. The transducer substrate of claim 15, wherein a reaction element of the plurality of reaction elements comprises NADH, and wherein the amount of fluorescence emitted from the chromophore is determined by a concentration of the NADH.

17. The transducer substrate of claim 15, wherein the analyte comprises NADH.

18. The transducer substrate according to claim 15, wherein the enzyme is an NADH-dependent or an NADPH-dependent enzyme.

19-21. (canceled)

22. The transducer substrate according to claim 15, wherein the enzyme is covalently bonded to the nanoparticle.

23. The transducer substrate according to claim 15, wherein the enzyme is not coupled to the nanoparticle.

24. (canceled)

25. The transducer substrate according to any of claim 15, wherein the enzyme is covalently bonded to the substrate.

26. The transducer substrate according to claim 15, wherein the nanoparticle is covalently bonded to the substrate.

27. The transducer substrate according to claim 15, wherein the enzyme is physically associated with the substrate.

28. The transducer substrate according of claim 27, wherein the enzyme and the nanoparticle are lyophilized onto the substrate.

29. The transducer substrate according to claim 15, wherein the enzyme is coupled to the substrate adjacent to the nanoparticle on a spot.

30. The transducer substrate of claim 29, wherein the enzyme is a first enzyme, the nanoparticle is a first nanoparticle, the chromophore is a first chromophore, and the reaction is a first reaction; and
wherein the transducer substrate further comprises:
a second nanoparticle comprising a second chromophore coupled to the substrate; and
a second enzyme different from the first enzyme coupled to the substrate configured to catalyze a second reaction comprising a second plurality of reaction elements;
wherein the second plurality of reaction elements comprise one or more second reactants including the analyte and one or more second products, and wherein an amount of fluorescence emitted from the second chromophore is determined by a concentration of a second reaction element of the second plurality of reaction elements.

31. The transducer substrate of claim 30, wherein the spot is a first spot, and wherein the second nanoparticle is coupled to the substrate on a second spot separate from the first spot.

32. The transducer substrate of claim 30, wherein the second nanoparticle is coupled to the substrate on the spot.

33. The transducer substrate of claim 30, wherein the first chromophore is configured to absorb light in a first absorption wavelength range and the second chromophore is configured to absorb light in a second absorption wavelength range different from the first absorption wavelength range.

34. The transducer substrate of claim 30, wherein the fluorescence emitted from the first chromophore is in a first emission wavelength range, and wherein fluorescence emitted from the second chromophore is in a second emission wavelength range different from the first emission wavelength range.

35. The transducer substrate of claim 30, wherein the second reaction is different from the first reaction.

36-39. (canceled)

40. The transducer substrate according to claim 29, wherein the substrate is configured to wick a fluid sample to the spot.

41. (canceled)

42. The transducer substrate according to claim 15, wherein the chromophore comprises a semiconducting polymer.

43-45. (canceled)

46. The transducer substrate according to claim 15, wherein the fluorescence emitted from the chromophore comprises a signal fluorescence wavelength and a control fluorescence wavelength, wherein the fluorescence emitted from the chromophore defines a signal fluorescence ratio equal to a ratio of an amount of fluorescence emitted at the signal fluorescence wavelength to an amount of fluorescence emitted at the control fluorescence wavelength, and wherein the signal fluorescence ratio is determined by the concentration of the reaction element of the plurality of reaction elements, and wherein the fluorescence ratio varies ratiometrically with the concentration of the analyte.

47. (canceled)

48. (canceled)

49. A kit for analyte concentration measurements, the kit comprising:
a nanoparticle comprising a chromophore; and
an enzyme physically associated with the nanoparticle and configured to catalyze a reaction comprising a plurality of reaction elements;
wherein the plurality of reaction elements comprises one or more reactants including the analyte and one or more products, and wherein an amount of fluorescence emitted from the chromophore is determined by a concentration of a reaction element of the plurality of reaction elements.

50-69. (canceled)

70. A transducer for analyte concentration measurements, the transducer comprising:
a chromophore including a semiconducting chromophoric polymer; and
an enzyme physically associated with the semiconducting chromophoric polymer and configured to catalyze a reaction comprising a plurality of reaction elements;
wherein the plurality of reaction elements comprises one or more reactants including the analyte and one or more products, and wherein an amount of fluorescence emitted from the chromophore is determined by a concentration of a reaction element of the plurality of reaction elements.

71-84. (canceled)

85. A system for analyte concentration measurements, the system comprising:
a transducer substrate of claim 15;
an illumination source configured to illuminate the chromophore of the nanoparticle transducer, the transducer substrate, the kit, or the transducer to induce fluorescence therefrom;
a photodetector configured to generate a signal based on the fluorescence from the chromophore; and
a controller operatively coupled to the illumination source and the photodetector and including logic, that when executed by the controller, causes the system to perform operations including:
illuminating the chromophore with the illumination source; and
determining a concentration of the analyte based upon the signal from the photodetector.

86-90. (canceled)

91. A method of measuring a concentration of an analyte in a fluid, the method comprising:
contacting the fluid with a Pdot including a chromophore and an NADH-dependent enzyme or NADPH-dependent enzyme coupled to the Pdot configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements;
illuminating the Pdot to induce fluorescence therefrom;
measuring the fluorescence from the Pdot; and
determining the concentration of the analyte based on the measured fluorescence.

92. A method of measuring a concentration of an analyte in a fluid, the method comprising:
contacting the fluid with a Pdot including a chromophore and an enzyme physically associated with the Pdot configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements;
illuminating the chromophore to induce fluorescence therefrom;
measuring the fluorescence from the chromophore; and
determining the concentration of the analyte based on the measured fluorescence.

93. A method of measuring a concentration of an analyte in a fluid, the method comprising:
contacting the fluid with chromophore including a semiconducting chromophoric polymer and an enzyme physically associated with the chromophore configured to catalyze a reaction comprising a plurality of reaction elements, wherein the plurality of reaction elements comprises one or more reactants including the analyte and one or more products, and wherein an amount of fluorescence emitted from the chromophore is determined by a concentration of a reaction element of the plurality of reaction elements;
illuminating the chromophore to induce fluorescence therefrom;
measuring the fluorescence from the chromophore; and
determining the concentration of the analyte based on the measured fluorescence.

94-109. (canceled)
